(19) United States
(12) Patent Application Publication
(10) Pub. No.: US 2026/0308516 A1
(43) Pub. Date: Oct. 8, 2026

(54) METABOLIC SWITCHES FOR ANTI-TUMOR IMMUNITY

(71) Applicant: Cold Spring Harbor Laboratory, Cold Spring Harbor, NY (US)

(72) Inventors: Semir Beyaz, Cold Spring Harbor, NY (US); Ilgin Ergin, Cold Spring Harbor, NY (US); Timothy Maher, Cold Spring Harbor, NY (US); Paul Bunk, Cold Spring Harbor, NY (US); Vyom Shah, Cold Spring Harbor, NY (US); Charlie Chung, Cold Spring Harbor, NY (US)

(73) Assignee: Cold Spring Harbor Laboratory, Cold Spring Harbor, NY (US)

(21) Appl. No.: 19/476,772

(22) PCT Filed: Apr. 19, 2024
(86) PCT No.: PCT/US2024/025353

§ 371 Date: Oct. 20, 2025

Related U.S. Application Data
(60) Provisional application No. 63/460,971, filed on Apr. 21, 2023.

(51) Int. Cl. C12N 5/0783; A61K 40/11; A61K 40/31; A61K 40/42; A61P 35/00; C07K 14/005; C07K 14/475; C07K 16/32; C12N 9/10

(52) U.S. Cl. CPC C12N 5/0636; A61K 40/11; A61K 40/31; A61K 40/4211; A61P 35/00; C07K 14/005; C07K 14/475; C07K 16/32; C12N 9/1029; C12Y 203/01021; C07K 2319/00; C12N 2510/00; C12N 2710/16622

(57) ABSTRACT

Provided herein are fusion proteins comprising a peroxisome proliferator-activated receptor delta (PPAR-5) sequence linked to an activation domain sequence for increasing immune cell activity against cancer cells and also provided herein are CPT1A proteins for increasing immune cell activity against cancer cells.

### CROSS-REFERENCE TO RELATED APPLICATIONS

This application claims the benefit under 35 U.S.C. § 119(e) of U.S. Provisional Application No. 63/460,971, filed Apr. 21, 2023, entitled “METABOLIC SWITCHES FOR ANTI-TUMOR IMMUNITY,” the entire disclosure of which is hereby incorporated by reference in its entirety.

### REFERENCE TO AN ELECTRONIC SEQUENCE LISTING

The contents of the electronic sequence listing (C130070040WO00-SEQ-FL.xml; Size: 58,971 bytes; and Date of Creation: Apr. 3, 2024) is herein incorporated by reference in its entirety.

### BACKGROUND

Immunotherapy is one of the mainstays of personalized medicine. The development of chimeric antigen receptor (CAR) T-cell therapy has revolutionized the treatment of numerous blood cancers including lymphomas, multiple myeloma, and some forms of leukemia. Engineering of a patient's T cells to include a CAR increases tumor-specific cytotoxicity while lowering the risk of graft rejection. Unfortunately, cancer recurrence occurs in over half of patients treated with CAR-T therapy and some patients suffer debilitating sides effects due to off-target effects on non-cancerous cells. Furthermore, immunotherapy has shown limited efficacy in the treatment of non-hematological malignancies.

### SUMMARY

While the development of immunotherapy has increased the arsenal of cancer drugs, existing methods of immunotherapy are limited by cancer recurrence and a lack of efficacy against non-hematological malignancies. In particular, T cells comprising a chimeric antigen receptor (CAR) have reduced efficacy over time as constitutive signaling through the CAR can lead to exhaustion. The compositions and methods disclose herein address many of these limitations.

The present disclosure is based, at least in part, on the unexpected finding that constitutive activation of peroxisome proliferator-activated receptor delta (PPAR-δ) is sufficient to increase T-cell infiltration and T cell-mediated cytotoxicity in different mouse and human models of cancer. Without being bound by a particular theory, existing methods of activating PPAR-δ rely on ligand binding, which may be subject to inactivation through negative feedback inhibition. Notably, as proof of concept, introduction of a fusion protein comprising (a) PPAR-δ linked to (b) a Herpes simplex virion protein 16 (VP16) activation domain into HER2 CAR-T cells initiated killing of a HER2 amplified human ovarian cancer cells in less than 30 hours and significantly increased the cytotoxicity of the HER2 CAR-T cells against the cancer cells as compared to results with HER2 CAR-T cells without the PPAR-δ fusion protein. Introduction of the VP16-PPAR-δ fusion protein into immune cells also did not alter histology of tissues including lung, heart, liver, kidney, small intestine, colon, thymus, and spleen in mouse models. Furthermore, the VP16-PPAR-δ fusion protein did not elicit an inflammatory response. The results were surprising at least in part because some studies have linked PPAR-δ expression or activation with a decrease in proliferation of thymocytes (immature T cells) and endothelial cell proliferation. See, e.g., Mothe-Satney et al. Sci Rep, 2016; 6:34317 and Piqueras et al., Arterioscler Thromb Vasc Biol. 2007 January; 27(1):63-9. Numerous studies also suggest PPARs and their downstream regulators suppress immunity and promote tumor growth (Beyaz et al., 2016 Nature 531, 53-58 and Neels and Grimaldi, 2014 Physiol Rev 94, 795-858). Furthermore, the efficacy of many existing immunotherapy drugs is limited to only a subset of patients with a particular tumor mutation profile or immune infiltration status. In contrast, the results herein demonstrate that expression of the VP16-PPAR-δ fusion protein in immune cells increased the elimination of cancer cells irrespective of the cancer cells' mutation status and irrespective of the extent of immune cell infiltration.

PPAR-δ along with PPAR-α and PPAR-γ are members of the PPAR family of transcription factors that have been shown to promote expression of target genes in a ligand-dependent manner. The PPARs regulate different aspects of energy homeostasis and metabolic function in cells. For example, PPAR-α activation has been implicated in reduction of triglyceride levels. PPAR-γ activation can increase insulin sensitization and glucose metabolism. In contrast, activation of PPAR-δ has been shown to enhance fatty acids metabolism. See, e.g., Kliewer et al. Recent Prog Horm Res. 2001; 56:239-63.

Although all three PPARs comprise a DNA binding domain and a ligand-binding domain, there is relatively high structural divergence between the ligand-binding domains of the three PPARs within a given species. For example, the amino acid sequence of the ligand-binding domains of PPAR-α, PPAR-δ, and PPAR-γ within a species is only about 65% identical. See, e.g., Juge-Aubry et al., J Biol Chem. 1997 Oct. 3; 272(40):25252-9.

Given the ligand-dependent mechanism of action of PPARs, existing methods of PPAR activation rely on small molecule agonists. The agonists, however, lack specificity and have off-target activity. For example, although designed to target PPAR-δ, GW501516 is a small molecule that activates both PPAR-α and PPAR-δ. Since the ligand-binding domain of PPARs are quite divergent, GW501516 likely affects other targets. Furthermore, there is concern that GW501516 induces endothelial proliferation and angiogenesis. See, e.g., Piqueras et al., Arterioscler Thromb Vasc Biol. 2007 January; 27(1):63-9.

Surprisingly, the methods described herein show that introduction of a VP16-PPAR-δ fusion protein increases immune cell-mediated toxicity against tumor cells without activation of PPAR-α and PPAR-γ.

Aspects of the present disclosure provide methods of engineering an immune cell comprising introducing into an immune cell a fusion protein that comprises or consists of: (a) a peroxisome proliferator-activated receptor delta (PPAR-δ) sequence linked to (b) an activation domain sequence.

In some embodiments, the PPAR-δ sequence comprises or consists of a DNA binding domain and a ligand-binding domain.

In some embodiments, the PPAR-δ sequence comprises or consists of a sequence that is at least 90% identical to SEQ ID NO: 21 or SEQ ID NOs: 24-26.

In some embodiments, the activation domain sequence is a Herpes simplex virion protein 16 (VP16) activation domain sequence.

In some embodiments, the methods comprise introducing a nucleic acid sequence encoding the fusion protein.

In some embodiments, the nucleic acid sequence is present on an expression vector.

In some embodiments, the expression vector is a viral vector.

In some embodiments, the PPAR-δ sequence is linked to the VP16 activation domain sequence via a peptide linker.

In some embodiments, the peptide linker is a poly-Glycine-Serine (G4S) linker.

In some embodiments, the immune cell is a lymphoid cell or a myeloid cell.

In some embodiments, the immune cell is a myeloid cell.

In some embodiments, the myeloid cell is a macrophage.

In some embodiments, the immune cell is a lymphoid cell.

In some embodiments, the lymphoid cell is a T-cell or natural killer cell.

In some embodiments, the T-cell is a regulatory T cell or a cytotoxic T cell.

In some embodiments, the T-cell comprises a chimeric antigen receptor (CAR).

In some embodiments, the methods comprise introducing a chimeric antigen receptor (CAR) into the T-cell.

In some embodiments, the CAR comprises a HER-2 antibody.

In some embodiments, the VP16 activation domain sequence comprises four copies of VP16.

In some embodiments, the VP16 activation domain sequence is VP64.

In some embodiments, the fusion protein comprises or consists of an amino acid sequence that is at least 90% identical to SEQ ID NO: 1, 14, 15, or 16.

In some embodiments, the VP16 activation domain sequence is linked to the N-terminus of the PPAR-δ sequence.

In some embodiments, the fusion protein is introduced in an amount sufficient to increase expression of carnitine palmitoyltransferase 1A (CPT1A) by at least 25% as compared to the immune cell without the fusion protein.

In some embodiments, the fusion protein is introduced in an amount sufficient to increase expression of expression of a proinflammatory molecule selected from the group consisting of Isg15, irf7, IRF1, ifit3, ifi208, and cxcl10 by at least 25% as compared to the immune cell without the fusion protein.

In some embodiments, the fusion protein is introduced in an amount sufficient to increase expression of CXCR3, CXCR1, CCR6, GZMK, GZMB, GZMF, HIST4H4, HIST1H4M, BCL2, FOS, and/or JUN by at least 25% as compared to the immune cell without the fusion protein.

In some embodiments, the fusion protein is introduced in an amount sufficient to decrease expression of PDCD1, TIM-3 and LAG-3 by at least 25% as compared to the immune cell without the fusion protein.

In some embodiments, the fusion protein is introduced in an amount sufficient to increase expression of TNF-α by at least 25% as compared to the immune cell without the fusion protein.

In some embodiments, the fusion protein is introduced in an amount sufficient to decrease expression of FOXP3 by at least 25% and/or increase expression of IFNG and TBX21 (t-bet) by at least 25% as compared to the immune cell without the fusion protein.

In some embodiments, the immune cell expresses CD8 and/or CXCR3.

In some embodiments, the immune cell comprises a nucleotide sequence encoding CPT1A and/or CXCR3.

In some embodiments, the fusion protein is introduced in an amount sufficient to increase infiltration of the immune cell into a tumor and/or increases the lifespan of the immune cell as compared to an immune cell that does not comprise the fusion protein.

Further aspects of the present disclosure provide methods of increasing T cell-mediated cytotoxicity comprising introducing into a T cell a fusion protein that comprises: (a) a peroxisome proliferator-activated receptor delta (PPAR-δ) linked to (b) an activation domain sequence.

In some embodiments, the PPAR-δ sequence comprises or consists of a DNA binding domain and a ligand-binding domain.

In some embodiments, the PPAR-δ sequence comprises or consists of a sequence that is at least 90% identical to SEQ ID NO: 21 or SEQ ID NOs: 24-26.

In some embodiments, the activation domain sequence is a Herpes simplex virion protein 16 (VP16) activation domain sequence.

In some embodiments, the methods comprise introducing a nucleic acid sequence encoding the fusion protein.

In some embodiments, the nucleic acid sequence is present on an expression vector.

In some embodiments, the expression vector is a viral vector.

In some embodiments, the PPAR-δ sequence is linked to the VP16 activation domain sequence via a peptide linker.

In some embodiments, the peptide linker is a poly-Glycine-Serine (G4S) linker.

In some embodiments, the T-cell is a regulatory T cell or a cytotoxic T cell.

In some embodiments, the T-cell comprises a chimeric antigen receptor (CAR).

In some embodiments, the methods comprise introducing a chimeric antigen receptor (CAR) into the T-cell.

In some embodiments, the CAR comprises a HER-2 antibody.

In some embodiments, the VP16 activation domain sequence comprises four copies of VP16.

In some embodiments, the VP16 activation domain sequence is VP64.

In some embodiments, the fusion protein comprises or consists of an amino acid sequence that is at least 90% identical to SEQ ID NO: 1, 14, 15, or 16.

In some embodiments, the VP16 activation domain sequence is linked to the N-terminus of the PPAR-δ sequence.

In some embodiments, the fusion protein is introduced in an amount sufficient to increase expression of carnitine palmitoyltransferase 1A (Cpt1A) by at least 25% as compared to the T-cell without the fusion protein.

In some embodiments, the fusion protein is introduced in an amount sufficient to increase expression of CXCR3, CXCR1, CCR6, GZMK, GZMB, GZMF, HIST4H4, HIST1H4M, BCL2, FOS, and/or JUN by at least 25% as compared to the T-cell without the fusion protein.

In some embodiments, the fusion protein is introduced in an amount sufficient to decrease expression of Pdcd1, TIM-3 and Lag-3 by at least 25% as compared to the T-cell without the fusion protein.

In some embodiments, the fusion protein is introduced in an amount sufficient to increase expression of TNF-α by at least 25% as compared to the T-cell without the fusion protein.

In some embodiments, the fusion protein is introduced in an amount sufficient to decrease expression of FOXP3 by at least 25% and/or increase expression of IFNG and TBX21 (t-bet) by at least 25% as compared to the T-cell without the fusion protein.

In some embodiments, the T-cell expresses CD8 and/or CXCR3.

In some embodiments, the T-cell comprises a nucleotide sequence encoding CPT1A and/or CXCR3.

In some embodiments, the fusion protein is introduced in an amount sufficient to increase infiltration of the T-cell into a tumor and/or increases the lifespan of the T-cell as compared to a T-cell that does not comprise the fusion protein.

Further aspects of the present disclosure provide methods of treating a subject with cancer comprising administering to the subject an immune cell comprising a fusion protein that comprises: (a) a peroxisome proliferator-activated receptor delta (PPAR-δ) linked to (b) an activation domain sequence.

In some embodiments, the PPAR-δ sequence comprises or consists of a DNA binding domain and a ligand-binding domain.

In some embodiments, the PPAR-δ sequence comprises or consists of a sequence that is at least 90% identical to SEQ ID NO: 21 or SEQ ID NOs: 24-26.

In some embodiments, the activation domain sequence is a Herpes simplex virion protein 16 (VP16) activation domain sequence.

In some embodiments, the cancer is melanoma, breast cancer, colon cancer, or ovarian cancer.

In some embodiments, the immune cell is introduced in a number sufficient to decrease the size of a tumor in the subject by at least 25% as compared to when the immune cell is not introduced to the subject.

In some embodiments, the immune cell is introduced in a number sufficient to increase the number of tumor-infiltrating immune cells in a tumor of the subject by 25% as compared to when the immune cell is not introduced to the subject.

In some embodiments, the immune cell is introduced in a number sufficient to decrease the rate of metastasis in a subject by 25% as compared to when the immune cell is not introduced to the subject.

In some embodiments, the method further comprises administering an immune checkpoint inhibitor to the subject.

In some embodiments, the immune checkpoint inhibitor is a PD-1 inhibitor, CTLA-4 inhibitor, or a PD-L1 inhibitor.

In some embodiments, the immune checkpoint inhibitor is an antibody.

Further aspects of the present disclosure provide methods of engineering an immune cell comprising introducing into an immune cell a CPT1A protein.

Further aspects of the present disclosure provide methods of treating a subject with cancer comprising administering to the subject an immune cell comprising a CPT1A protein.

In some embodiments, the methods comprise introducing a nucleic acid sequence encoding the CPT1A protein.

In some embodiments, the nucleic acid sequence is present on an expression vector.

In some embodiments, the expression vector is a viral vector.

In some embodiments, the immune cell is a lymphoid cell or a myeloid cell.

In some embodiments, the immune cell is a myeloid cell.

In some embodiments, the myeloid cell is a macrophage.

In some embodiments, the immune cell is a lymphoid cell.

In some embodiments, the lymphoid cell is a T-cell or natural killer cell.

In some embodiments, the T-cell is a regulatory T cell or a cytotoxic T cell.

In some embodiments, the T-cell comprises a chimeric antigen receptor (CAR).

In some embodiments, the methods comprise introducing a chimeric antigen receptor (CAR) into the T-cell.

In some embodiments, the CAR comprises a HER-2 antibody.

In some embodiments, the CPT1A protein comprises an amino acid sequence that is at least 90% identical to SEQ ID NO: 12.

In some embodiments, the immune cell expresses CD8 and/or CXCR3.

In some embodiments, the immune cell comprises a nucleotide sequence encoding or CXCR3.

In some embodiments, the fusion protein is introduced in an amount sufficient to increase infiltration of the immune cell into a tumor and/or increases the lifespan of the immune cell as compared to an immune cell that does not comprise the fusion protein.

In some embodiments, the cancer is melanoma, breast cancer, colon cancer, or ovarian cancer.

In some embodiments, the immune cell is introduced in number sufficient to decrease the size of a tumor in the subject by at least 25% as compared to when the immune cell is not introduced to the subject.

In some embodiments, the immune cell is introduced in a number sufficient to increase the number of tumor-infiltrating immune cells in a tumor of the subject by 10% as compared to when the immune cell is not introduced to the subject.

Further aspects of the present disclosure provide methods of increasing T cell-mediated cytotoxicity comprising introducing into a T-cell a CPT1A protein.

In some embodiments, the methods comprise introducing a nucleic acid sequence encoding the CPT1A protein.

In some embodiments, the nucleic acid sequence is present on an expression vector.

In some embodiments, the expression vector is a viral vector.

In some embodiments, the T-cell comprises a chimeric antigen receptor (CAR).

In some embodiments, the methods comprise introducing a chimeric antigen receptor (CAR) into the T-cell.

In some embodiments, the CAR comprises a HER-2 antibody.

In some embodiments, the CPT1A protein comprises an amino acid sequence that is at least 90% identical to SEQ ID NO: 12.

In some embodiments, the immune cell expresses CD8 and/or CXCR3.

In some embodiments, the immune cell comprises a nucleotide sequence encoding or CXCR3.

In some embodiments, the fusion protein is introduced in an amount sufficient to increase infiltration of the T-cell into a tumor and/or increases the lifespan of the immune cell as compared to an T-cell that does not comprise the fusion protein.

Further aspects of the present disclosure provide a T-cell comprising: (a) a chimeric antigen receptor and (b) a fusion protein that comprises or consists of: (i) a peroxisome proliferator-activated receptor delta (PPAR-δ) linked to (ii) an activation domain sequence.

Further aspects of the present disclosure provide a T-cell comprising: (a) a chimeric antigen receptor and (b) a fusion protein that comprises or consists of: (i) a peroxisome proliferator-activated receptor delta (PPAR-δ) linked to (ii) an activation domain sequence.

Further aspects of the present disclosure provide a T-cell comprising: (a) a chimeric antigen receptor and (b) an engineered polynucleotide encoding (i) a peroxisome proliferator-activated receptor delta (PPAR-δ) linked to (ii) an activation domain sequence or (c) a chimeric antigen receptor and (d) an engineered polynucleotide encoding a CPT1A.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. For purposes of clarity, not every component may be labeled in every drawing.

FIGS. 1A-1N show PPARδ activation promotes anti-tumor immunity. FIGS. 1A-AC show subcutaneous tumor growth curves of control (wild-type, “WT”) and vav-cre+ VP16-PPARδ+ mice inoculated with 3×10⁵ MC38 (a), 2.5×10⁵ B16-F10 (b), or 2.5×10⁵ EO771 (c) cells. FIG. 1D is a representative colonoscopy image of control (wild-type, “WT”) and vav-cre+ VP16-PPARδ+ mice 5 weeks after orthotopic injection of AKP [APC^{null}, Kras^{G12D}, p53^{null}, (Roper et al., 2017) organoids. FIG. 1E shows the Orthotopic tumor growth curve of control (wild-type, “WT”) and vav-cre+ VP16-PPARδ+ mice inoculated with AKP organoids. Tumor index was calculated by dividing the tumor diameter by the colon diameter. Each tumor index was normalized to their respective week-1 tumor index. FIG. 1F shows the survival curve of control (wild-type, “WT”) and vav-cre+ VP16-PPARδ+ mice inoculated with AKP organoids n≥9. FIG. 1G is a representative H&E image of week-2 orthotopic AKP tumors grown in control (wild-type, “WT”) and vav-cre+ VP16-PPARδ+ mice. FIG. 1H shows the quantification of total tumor area from H&E images of week-2 and week-3 orthotopic AKP tumors grown in control (wild-type, “WT”) and vav-cre+ VP16-PPARδ+ mice n≥5. FIG. 1I is a Representative pan-keratin, CD45 and DAPI immunofluorescence image of week-2 orthotopic AKP tumors grown in control (wild-type, “WT”) and vav-cre+ VP16-PPARδ+ mice. FIG. 1J shows the quantification of total CD45+ cells normalized to % pan-keratin positive area in AKP tumors grown in WT and vav-cre+ VP16-PPARδ+ mice. FIGS. 1K-1N show PPARS activation promotes anti-tumor immunity and reduces metastasis rate in immunosuppressive metastatic colorectal carcinoma. FIG. 1K includes representative colonoscopy images of mice inoculated with APC^{null} Kras^{G12D} p53^{null} Smad4^{null} (AKPS) (see, e.g., Westcott et al., Nat. Cancer, 2021) organoids into colon sub-mucosa using colonoscopy guided orthotopic injections. Wild-type mice were sub-lethally irradiated with 5Gy total body radiation. 2 days later, mice were intravenously injected with PBS, 5×10⁶ wild-type PBMCs, or 5×10⁶ VP16-PPARS PBMCs. 3 days later, mice were orthotopically injected with intact AKPS organoids that contain approximately 2×10⁶ single cells. Colonoscopy images were taken 3 weeks after tumor inoculation. PBMC: peripheral blood mononuclear cells. FIG. 1L shows a survival curve of AKPS tumor bearing mice that received PBS, wild-type PBMCs, or VP16-PPARδ PBMCs prior to tumor inoculation. FIG. 1M shows AKPS tumor weights at humane end points (40-60 days post tumor inoculation). n=4 PBS, n=5 WT PBMC, n=4 VP16-PPARδ. FIG. 1N shows the rate of lung, liver or omentum metastases in AKPS tumor bearing mice that received PBS, wild-type PBMCs, or VP16-PPARδ PBMCs prior to tumor inoculation.

FIGS. 2A-2F show PPARδ activation in immune cells remodels the tumor microenvironment into a pro-inflammatory state. FIG. 2A shows a Uniform Manifold Approximation and Projection (UMAP) visualization of single cell RNA sequencing data analysis of day-12 subcutaneous MC38 tumors isolated from control (wild-type, “WT”) and vav-cre+ VP16-PPARδ+ mice. FIG. 2B is bubble plot visualizing log₂ fold changes and −log(p) values of the average expression of genes involved in PPAR signaling, interferon signaling, MHC/antigen presentation, chemoattraction and survival across tumor infiltrating immune cell clusters. FIG. 2C shows a hallmark inflammatory response z-scores across control (wild-type) and vav-cre+ VP16-PPARδ+ tumor infiltrating immune cell clusters. FIGS. 2D-2E show the density log fold change UMAP of Irf7 (FIG. 2D) and Ccl2 (FIG. 2E) gene expression across vav-cre+ VP16-PPARδ+ tumor infiltrating immune cells. FIG. 2F shows the Pearson Correlation Coefficient (R) and p values demonstrating the correlation between “immune response”, “PPAR induced”, hallmark inflammatory response and KEGG PPAR signaling z-scores in human melanoma tumor infiltrating immune cells.

FIGS. 3A-3K show cooperative interactions between pro-inflammatory myeloid cells and cytotoxic CD8 T cells mediate PPARδ driven boost in anti-tumor immunity FIG. 3A shows a summary of ligand-target interactions identified using NicheNet analysis (Browaeys et al., 2020). FIG. 3B is a split violin plots demonstrating the expression levels of key genes involved in CD8 T cell migration, effector function, survival and dysfunction in control (wild-type, “WT”) and vav-cre+ VP16-PPARδ+ tumor infiltrating CD8 T cells. FIG. 3C is a bubble plot demonstrating the log₂ fold changes and −log(p) values of genes involved in migration, interferon response, T cell activation, dysfunction and survival in CD8 T cell and exhausted CD8 T cell clusters in control (wild-type, “WT”) and vav-cre+ VP16-PPARδ+ tumor infiltrating cells. FIG. 3D shows day-25 tumor weights of subcutaneous MC38 tumors grown in vav-cre+ VP16-PPARδ+ mice upon in vivo CD8a+ cell depletion, compared to tumors grown in IgG control treated control (wild-type, “WT”) and vav-cre+ VP16-PPARδ+ mice. FIG. 3E shows the Pearson Correlation Coefficient (R) and p values demonstrating the correlation between “PPAR induced” and Ifng, Irf7 and Cxcr3 z-scores in human melanoma tumor infiltrating T cells. FIG. 3F is a heatmap demonstrating log₂ fold changes of top upregulated genes in vav-cre+ VP16-PPARδ+ tumor infiltrating myeloid cell clusters. FIG. 3G shows a subcutaneous tumor growth curve of control (wild-type, “WT”) and LysM-cre+ VP16-PPARδ+ mice inoculated with 3×10⁵ MC38 cells. FIG. 3H shows day-25 tumor weights of subcutaneous MC38 tumors grown in control (wild-type, “WT”) and LysM-cre+ VP16-PPARδ+ mice. FIG. 3I is a representative CD3, CD8 and DAPI immunofluorescence image of day-25 subcutaneous MC38 tumors grown in control (wild-type, “WT”) and LysM-cre+ VP16-PPARδ+ mice. FIG. 3J shows the quantification of CD3+ CD8+ cells per area using CD3, CD8 and DAPI immunofluorescence images of day-25 subcutaneous MC38 tumors grown in control (wild-type, “WT”) and LysM-cre+ VP16-PPARδ+ mice (n=3). FIG. 3K shows day-25 tumor weights of subcutaneous MC38 tumors grown in of control (wild-type, “WT”) and LysM-cre+ VP16-PPARδ+ mice upon in vivo CD8a+ cell depletion.

FIGS. 4A-4I show PPARδ target Cpt1a in part mediates the effects of PPARδ on anti-tumor immunity. FIG. 4A shows Pearson Correlation Coefficient (R) and p values demonstrating the correlation between “PPAR induced”, and “GO fatty acid oxidation” z-scores in human melanoma tumor infiltrating immune cells. FIG. 4B shows subcutaneous tumor growth curves of control (wild-type, “WT”) and vav-cre+ Cpt1a^{fl/fl} mice inoculated with 3×10⁵ MC38 cells. FIG. 4C shows day-25 tumor weights of subcutaneous MC38 tumors grown in of control (wild-type, “WT”) and vav-cre+ Cpt1a^{fl/fl} mice upon in vivo CD8a+ cell depletion. FIG. 4D shows hallmark inflammatory response z-scores across control (wild-type, “WT”) and vav-cre+ Cpt1a^{fl/fl} tumor infiltrating immune cell clusters. FIG. 4E is a density log fold change UMAP of Irf1 gene expression log₂ fold change across vav-cre+ Cpt1a^{fl/fl} tumor infiltrating immune cells. FIG. 4F shows bulk RNA sequencing analysis of CD8 T cells sorted from tumor draining inguinal lymph nodes of MC38 tumor-bearing control (wild-type, “WT”) and vav-cre+ Cpt1a^{fl/fl} mice, 12 days after tumor injection. FIG. 4G shows subcutaneous tumor growth curves of control, vav-cre+ VP16-PPARδ+, and vav-cre+ VP16-PPARδ+ Cpt1a^{fl/fl} mice inoculated with 3×10⁵ MC38 cells. FIG. 4H is a representative CD3, CD8 and DAPI immunofluorescence image of day-25 subcutaneous MC38 tumors grown in control, vav-cre+ VP16-PPARδ+, and vav-cre+ VP16-PPARδ+ Cpt1a^{fl/fl} mice. FIG. 4I shows quantification of CD3+ CD8+ cells per unit area from immunofluorescence images of day-25 subcutaneous MC38 tumors grown in control, vav-cre+ VP16-PPARδ+, and vav-cre+ VP16-PPARδ+ Cpt1a^{fl/fl} mice (n=3).

FIGS. 5A-5G show that cell intrinsic PPARδ activation is sufficient to enhance human CAR-T cell cytotoxicity. FIG. 5A is a VP64-PPARδ expressing human HER2 CAR-T cell construct design. FIG. 5B shows the PPARδ mRNA quantification of wild-type or VP64-PPARδ expressing HER2 CAR-T cells determined by qRT-PCR. FIG. 5C shows impedance-based real-time cell analysis (RTCA) demonstrating HER2 CAR-T cell mediated killing of HER2 overexpressing SKOV3 cells at effector-to-target ratios 10:1 and 2:1. FIGS. 5D-5E show an intracellular flow cytometry analysis of (FIG. 5D) IFNg and (FIG. 5E) TNFa production by primary murine CD8 T cells that were treated with vehicle or GW501516 during in vitro T cell activation, n≥8, 3 independent experiments with 3 technical replicates, unpaired t test, ****p≤0.0001, ±SD. FIG. 5F shows PPARδ mRNA quantification of wild-type or VP64-PPARδ expressing HER2 CAR-T cells determined by qRT-PCR, n≥2, unpaired t test, * p≤0.05, ±SD. FIG. 5G shows a PCA plot of wild-type and VP64-PPARδ expressing HER2 CAR-T cells. Results for wild-type HER2 CAR-T cells clustered on the left side of the graph and results for VP64-PPARδ expressing HER2 CAR-T cells clustered on the right side of the graph.

FIGS. 6A-6J include data showing VP16-PPARδ-mediated antitumor immunity in a xenograft model of cancer. FIG. 6A shows PPARδ mRNA quantification of vav-cre+ PPARδ^{fl/fl} splenic CD8 T cells using quantitative reverse-transcription polymerase chain reaction (qRT-PCR). FIG. 6B shows anti-PPARδ and anti-VP16 immunoblots of splenic PBMCs isolated from control, vav-cre+ PPARδ^{fl/fl} and vav-cre+ VP16-PPARδ+ mice. FIG. 6C shows subcutaneous tumor growth curve of control (wild-type, “WT”) and vav-cre+ vav-cre+ PPARδ^{fl/fl} mice inoculated with 3×10⁵ MC38 cells. FIG. 6D shows day-25 tumor weights of subcutaneous MC38 tumors grown in control (wild-type, “WT”) and vav-cre+ PPARδ^{fl/fl} mice. FIG. 6E shows day-25 tumor weights of subcutaneous MC38 tumors grown in control (wild-type, “WT”) and vav-cre+ VP16-PPARδ+ mice. FIGS. 6F-6H are representative H&E images of day-25 subcutaneous MC38 (FIG. 6F), B16-F10 (FIG. 6G), or EO771 (FIG. 6H) tumors grown in control (wild-type, “WT”) and vav-cre+ VP16-PPARδ+ mice. FIG. 6I is a representative CD45 and DAPI immunofluorescence image of day-25 subcutaneous MC38 tumors grown in control (wild-type, “WT”) and vav-cre+ VP16-PPARδ+ mice. FIG. 6J shows quantification of CD45+ cells per unit area from immunofluorescence images of day-25 subcutaneous MC38 tumors grown in control (wild-type, “WT”) and vav-cre+ VP16-PPARδ+ mice (n=3).

FIGS. 7A-7I include data showing that expression of VP16-PPARδ in immune cells increased the survival of mice with orthotopic MC38 tumors. FIG. 7A is a schematic describing colonoscopy-guided colorectal cancer injections. FIG. 7B shows a representative colonoscopy image of control (wild-type, “WT”) and vav-cre+ VP16-PPARδ+ mice 3 weeks after orthotopic injection of 5×10⁶ MC38 cells. FIG. 7C shows orthotopic tumor growth curve of control (wild-type, “WT”) and vav-cre+ VP16-PPARδ+ mice inoculated with MC38 tumors. Tumor index was calculated by dividing the tumor diameter by the colon diameter. Each tumor index was normalized to their respective week-1 tumor index. FIG. 7D shows a survival curve of control (wild-type, “WT”) and vav-cre+ VP16-PPARδ+ mice inoculated with orthotopic MC38 tumors, n≥9. FIG. 7E shows a quantification of CD45+ cells per unit area from immunofluorescence images of week-2 and week-3 orthotopic AKP tumors grown in control (wild-type, “WT”) and vav-cre+ VP16-PPARδ+ mice, n=3. FIG. 7F shows quantification of pan-keratin+ area from immunofluorescence images of week-2 and week-3 orthotopic AKP tumors grown in control (wild-type, “WT”) and vav-cre+ VP16-PPARδ+ mice, n=3. FIG. 7G is a representative CD45, pan-keratin and DAPI immunofluorescence image of week-2 orthotopic AKP tumors grown in control (wild-type, “WT”) and vav-cre+ VP16-PPARδ+ mice, arrows indicate immune hubs observed in vav-cre+ VP16-PPARδ+ mice. FIG. 7H shows the quantification of immune hub area from CD45, pan-keratin and DAPI immunofluorescence images of week-2 and week-3 orthotopic AKP tumors grown in control (wild-type, “WT”) and vav-cre+ VP16-PPARδ+ mice, n=3. FIG. 7I shows a 72-hour co-culture of WT, VP16-PPARδ+, or 72-hour GW501516 treated murine PBMCs with luciferase-expressing MC38 (MC38-luc) cells. Cancer cell lysis was calculated by adding luciferin at the endpoint and using luminescence as a proxy for MC38-luc cell abundance.

FIGS. 8A-8E include data showing single cell RNA sequencing results of MC38 tumors and analysis of tumor infiltrating immune cells from control (wild-type, “WT”) and vav-cre+ VP16-PPARδ+ mice. FIG. 8A is a schematic describing the experimental setup for single cell RNA sequencing of day-12 subcutaneous MC38 tumors isolated from control (wild-type, “WT”) and vav-cre+ VP16-PPARδ+ mice. FIG. 8B shows a bubble plot depicting the expression of immune cell type defining genes across tumor infiltrating immune cell clusters. FIG. 8C includes bar plots demonstrating the proportions of different immune cell clusters in control (wild-type, “WT”) and vav-cre+ VP16-PPARδ+ tumor infiltrating immune cells. FIG. 8D is a heatmap depicting log₂ fold changes of top differentially expressed genes across all vav-cre+ VP16-PPARδ+ tumor infiltrating immune cell clusters. FIG. 8E shows a density log fold change UMAP of Jun gene expression log₂ fold change across vav-cre+ VP16-PPARδ+ tumor infiltrating immune cells.

FIG. 9A-9B show hallmark IFNg (FIG. 9A) and IFNa (FIG. 9B) responses z-scores across tumor infiltrating immune cell clusters.

FIGS. 10A-10G include data showing that immune cell specific PPARδ activation does not lead to any adverse effects. FIG. 10A is a UMAP visualization of single cell RNA sequencing data analysis of splenocytes isolated from control (wild-type, “WT”) and vav-cre+ VP16-PPARδ+ mice at steady state. FIG. 10B includes bar plots demonstrating the proportions of different immune cell clusters in control (wild-type, “WT”) and vav-cre+ VP16-PPARδ+ splenocytes at steady state. FIG. 10C show bubble plot depicting the expression of immune cell type defining genes across splenocyte clusters at steady state. FIG. 10D shows hallmark inflammatory response z-scores across control (wild-type, “WT”) and vav-cre+ VP16-PPARδ+ splenocyte clusters at steady state. FIG. 10E show body weights of litter-mate control (wild-type, “WT”) and vav-cre+ VP16-PPARδ+ mice. FIG. 10F are flow cytometry analyses of control (wild-type, “WT”) and vav-cre+ VP16-PPARδ+ splenocytes at steady state. FIG. 10G show H&E staining of various tissues isolated from control (wild-type, “WT”) and vav-cre+ VP16-PPARδ+ at steady state.

FIGS. 11A-11I include data showing immunofluorescence analysis results of subcutaneous MC38 tumors grown in control (wild-type, “WT”) and vav-cre+ VP16-PPARδ+ mice using immune cell markers. FIG. 11A is a representative CD3, CD8 and DAPI immunofluorescence image of day-25 subcutaneous MC38 tumors grown in control (wild-type, “WT”) and vav-cre+ VP16-PPARδ+ mice. FIG. 11B shows the quantification of CD3+CD8+ cells per area using CD3, CD8 and DAPI immunofluorescence images of day-25 subcutaneous MC38 tumors grown in control (wild-type, “WT”) and vav-cre+ VP16-PPARδ+ mice (n=3). FIG. 11C is a split violin plots demonstrating the expression levels of key genes involved in CD8 T cell migration, effector function, survival and dysfunction in control (wild-type, “WT”) and vav-cre+ VP16-PPARδ+ tumor infiltrating exhausted CD8 T cell cluster. FIG. 11D is a heatmap demonstrating log₂ fold changes of top upregulated genes in vav-cre+ VP16-PPARδ+ tumor infiltrating CD8 T cell and exhausted CD8 T cell clusters. FIG. 11E show bulk RNA sequencing analysis of CD8 T cells sorted from tumor draining inguinal lymph nodes of MC38 tumor-bearing control (wild-type, “WT”) and vav-cre+ VP16-PPARδ+ mice, 12 days after tumor injection. FIG. 11F is a density log fold change UMAP of Cxcr3 gene expression log₂ fold change across vav-cre+ VP16-PPARδ+ tumor infiltrating immune cells. FIG. 11G shows day-25 tumor volumes of subcutaneous MC38 tumors grown in of control (wild-type, “WT”) and vav-cre+ VP16-PPARδ+ mice upon in vivo CXCR3+ cell depletion. FIG. 11H is a representative CD3, CD8 and DAPI immunofluorescence image of week-2 orthotopic AKP tumors grown in control (wild-type, “WT”) and vav-cre+ VP16-PPARδ+ mice. FIG. 11I shows the quantification of CD3+ CD8+ cells per area from immunofluorescence images of week-2 and week-3 orthotopic AKP tumors grown in control (wild-type, “WT”) and vav-cre+ VP16-PPARδ+ mice (n=3).

FIGS. 12A-12L include data showing that VP16-PPARδ expression increased TNFa secretion by primary murine CD8 T cells. FIG. 12A shows representative peripheral blood flow cytometry analysis demonstrating CD8 T cell depletion efficiency during in vivo CD8a+ depletion experiments. FIG. 12B shows representative peripheral blood flow cytometry analysis demonstrating CXCR3+ CD8 T cell depletion efficiency during in vivo CXCR3+ depletion experiments. FIGS. 12C-12E shows intracellular flow cytometry analysis of IL-2 (c), IFNg (d) and TNFa (e) production by primary murine CD8 T cells that were treated with vehicle or GW501516 during in vitro T cell activation (≥3 independent experiments). FIG. 12F shows intracellular flow cytometry analysis of TNFa production by primary murine CD8 T cells that were isolated from control (wild-type, “WT”) and vav-cre+ VP16-PPARδ+ mice and activated in vitro (3 independent experiments). FIG. 12G is a TNFa ELISA demonstrating TNFa secretion by primary murine CD8 T cells that were isolated from control (wild-type, “WT”) and vav-cre+ VP16-PPARδ+ mice and activated in vitro. FIGS. 12H-12I show the results of an in vitro co-culture experiment measuring CD8 T cell killing of MC38-luc cells after 72-hours co-culture using effector-to-target ratios 10:1 (FIG. 12H) or 2.5:1 (FIG. 12I). Prior to co-culture, primary murine CD8 T cells were isolated from control or vav-cre+ VP16-PPARδ+ mice and were activated in vitro. GW501516 treatment was performed during T cell activation. FIG. 12J shows day-21 MC38 tumor volumes upon in vivo CD8 T cell adoptive transfer. Wild-type mice were sub-lethally irradiated using 5 Gy whole-body radiation. 48 hours later, 3 million naïve splenic control or VP16-PPARδ+ CD8 T cells were intravenously transferred to recipient mice. 72 hours after adoptive transfer, 500,000 MC38 cells were subcutaneously injected and tumor growth was followed. FIG. 12K is a representative CD45 and DAPI immunofluorescence image of day-25 subcutaneous MC38 tumors grown in control (wild-type, “WT”) and LysM-cre+ VP16-PPARδ+ mice. FIG. 12L shows the quantification of CD45+ cells per unit area from immunofluorescence images of day-25 subcutaneous MC38 tumors grown in control (wild-type, “WT”) and LysM-cre+ VP16-PPARδ+ mice (n=3).

FIGS. 13A-13M include data showing the metabolic status of Cpt1a knockout (KO) CD8 T cells. FIG. 13A shows the qRT-PCR Cpt1a mRNA quantification of CD8 T cells isolated from control (wild-type, “WT”) and vav-cre+ Cpt1a^{fl/fl} mice. FIG. 13B is an anti-Cpt1a immunoblot of splenic PBMCs isolated from control (wild-type, “WT”) and vav-cre+ Cpt1a^{fl/fl} mice. FIGS. 13C-13D show mean fluorescent intensities (MFI) of MitoTracker Green (d) and TMRE (e) staining of CD8 T cells isolated from control (wild-type, “WT”) and vav-cre+ Cpt1a^{fl/fl} mice, determined by flow cytometry. FIG. 13E shows the oxygen consumption rate (OCR) during Agilent Seahorse Mito Stress test of CD8 T cells isolated from control (wild-type, “WT”) and vav-cre+ Cpt1a^{fl/fl} mice. FIG. 13F shows the hydrophilic metabolite profiling using high-performance liquid chromatography and high-resolution mass spectrometry and tandem mass spectrometry (HPLC-MS/MS) of CD8 T cells isolated from control, vav-cre+ VP16-PPARδ+ and vav-cre+ Cpt1a^{fl/fl} mice. FIGS. 13G-H show normalized carnitine peak areas of CD8 T cells isolated from control, vav-cre+ VP16-PPARδ+ and vav-cre+ Cpt1a^{fl/fl} mice, determined by HPLC-MS/MS. Peak areas were normalized by sum using MetaboAnalyst software. FIG. 13I shows day-25 tumor weights of subcutaneous MC38 tumors grown in control (wild-type, “WT”) and vav-cre+ Cpt1a^{fl/fl} mice. FIGS. 13J-13L show CD3e (FIG. 13J), CD8a (FIG. 13K), and IFNg (FIG. 13L) mRNA quantification using qRT-PCR. Bulk RNAs were isolated from day-25 MC38 tumors grown in control (wild-type, “WT”) and vav-cre+ Cpt1a^{fl/fl} mice using qRT-PCR. FIG. 13M shows the total ¹⁴C-palmitate oxidation of murine CD8 T cells. CD8 T cells isolated from control or vav-cre+ Cpt1a^{fl/fl} mice and activated in vitro for 72 hours. Prior to liquid scintillation counting, cells were incubated in KRBH medium in the presence of 0.8 mM L-carnitine, 2.5 mM glucose, and 0.25 mM [1-¹⁴C] palmitate for 3 hours. ¹⁴C-palmitate oxidation was measured as described herein in Example 5, n≥5, representative of 3 independent experiments, unpaired t test, ** p≤0.01, ±SD.

FIGS. 14A-14H include data showing the transcriptional changes in tumor infiltrating immune cells upon Cpt1a loss. FIG. 14A includes bar plots demonstrating the proportions of different immune cell clusters in control (wild-type, “WT”) and vav-cre+ Cpt1a^{fl/fl} tumor infiltrating immune cells. FIG. 14B is a heatmap depicting log₂ fold changes of top differentially expressed genes across all vav-cre+ Cpt1a^{fl/fl} tumor infiltrating immune cell clusters. FIG. 14C is a density log fold change UMAP of Bcl2 gene expression log₂ fold change across vav-cre+ Cpt1a^{fl/fl} tumor infiltrating immune cells. FIG. 14D shows the hallmark inflammatory response z-scores across control (wild-type, “WT”) and vav-cre+ Cpt1a^{fl/fl} tumor infiltrating immune cell clusters. FIG. 14E shows the bulk RNA sequencing analysis of CD8 T cells sorted from tumor draining inguinal lymph nodes of MC38 tumor-bearing control (wild-type, “WT”) and vav-cre+ Cpt1a^{fl/fl} mice, 12 days after tumor injection. FIG. 14F day-25 tumor weights of subcutaneous MC38 tumors grown in control, vav-cre+ VP16-PPARδ+, and vav-cre+ VP16-PPARδ+ Cpt1a^{fl/fl} mice. FIG. 14G is a representative CD45 and DAPI immunofluorescence image of day-25 subcutaneous MC38 tumors grown in control, vav-cre+ VP16-PPARδ+, and vav-cre+ VP16-PPARδ+ Cpt1a^{fl/fl} mice. FIG. 14H quantification of CD45+ cells per unit area from immunofluorescence images of day-25 subcutaneous MC38 tumors grown in control, vav-cre+ VP16-PPARδ+, and vav-cre+ VP16-PPARδ+ Cpt1a^{fl/fl} mice (n=3).

FIGS. 15A-15E show regulatory T cell specific PPARδ activation is sufficient to promote anti-tumor immunity. FIGS. 15A-15C show expression levels of Ifng (FIG. 15A), Tbx21/T-bet (FIG. 15B), Foxp3 (FIG. 15C) genes in control (wild-type, “WT”) and vav-cre+ VP16-PPARδ+ tumor infiltrating regulatory T cell clusters. FIG. 15D shows subcutaneous tumor growth curves of control (wild-type, “WT”) and Foxp3-cre+ VP16-PPARδ+ mice inoculated with 3×10⁵ MC38 cells. FIG. 15E shows the survival curve of control (wild-type, “WT”) and Foxp3-cre+ VP16-PPARδ+ mice inoculated with MC38 tumors.

FIG. 16 shows functional domains in wild-type PPARδ.

FIGS. 17A-17F show PPARδ activation in all immune cells promotes anti-tumor immunity. FIG. 17A shows an orthotopic tumor growth curve of control and vav-cre+ VP16-PPARδ+ mice inoculated with APC^{null} Kras^{G12D} p53^{null} Smad4^{null} (AKPS) organoids. Tumor index was calculated by dividing the tumor diameter by the colon diameter using colonoscopy images. Each tumor index was normalized to their respective week-1 tumor index, n≥6, unpaired t test, **p≤0.005, ±SEM. FIG. 17B shows representative colonoscopy images of mice inoculated with AKPS organoids into colon sub-mucosa using colonoscopy guided orthotopic injections. Wild-type mice were sub-lethally irradiated with 5Gy total body radiation. 2 days later, mice were intravenously injected with PBS, 5×10⁶ wild-type PBMCs, or 5×10⁶ VP16-PPARδ+ PBMCs. 3 days later, mice were orthotopically injected with AKPS organoids. Colonoscopy images were taken 3 weeks after tumor inoculation, PBMC: peripheral blood mononuclear cells. FIG. 17C shows AKPS tumor weights at endpoints (40-60 days post tumor inoculation), n≥4, unpaired t test, **p≤0.01, ±SEM. FIG. 17D shows percentage of lung, liver or omentum metastases in AKPS tumor bearing mice that received PBS, wild-type PBMCs, or VP16-PPARδ PBMCs prior to tumor inoculation, n≥4. FIG. 17E shows representative H&E images of AKPS tumors from mice that received PBS, wild-type PBMCs, or VP16-PPARδ PBMCs prior to tumor inoculation, scale bar=500 um. FIG. 17F shows representative H&E images of livers from mice that received PBS, wild-type PBMCs, or VP16-PPARδ PBMCs prior to tumor inoculation, scale bar=250 um,

FIGS. 18A-18B include data showing that PPARδ activation in the immune system drastically decreased the presence of fibrotic areas in AKP tumors. FIG. 18A shows representative Masson's trichrome images of week-3 orthotopic AKP tumors grown in control and vav-cre+ VP16-PPARδ+ mice, scale bars=200 um. FIG. 18B shows fibrosis scores of week-2 and week-3 orthotopic AKP tumors grown in control and vav-cre+ VP16-PPARδ+ mice, determined by Masson's trichrome stain. Fibrosis scores were calculated on the blue color channel, where maximum score was 300. N≥4, unpaired t test, ***p≤0.001, ±SEM.

FIGS. 19A-19B includes data showing that PPARδ activation in immune cells was sufficient to increase the number of tumor infiltrating lymphocytes (TILs) in AKPS tumors. FIG. 19A shows representative pan-keratin, CD45 and DAPI immunofluorescence images of orthotopic AKPS tumors grown in mice that received PBS, 5×10⁶ wild-type PBMCs, or 5×10⁶ VP16-PPARδ+ PBMCs before tumor implantation, scale bar=50 um. FIG. 19B shows quantification of total CD45+ cells per unit area from immunofluorescence images of orthotopic AKPS tumors grown in mice that received PBS, 5×10⁶ wild-type PBMCs, or 5×10⁶ VP16-PPARδ+ PBMCs before tumor implantation, n≥3, tumors were sliced in half at the median plane and sagittal serial sections were taken from both sides, 1-2 sections per tumor were imaged and ≥5 areas per tumor were quantified using QuPath, one-way ANOVA, **** p<0.0001, ±SD.

FIGS. 20A-20H include data showing the impact of PPARδ activation on clonal expansion of immune cells. FIG. 20A shows density log fold change UMAP across vav-cre+ VP16-PPARδ+ tumor infiltrating immune cells in AKPS tumors. FIG. 20B shows relative abundance of clonal T cells in control and vav-cre+ VP16-PPARδ+ tumor infiltrating immune cells in AKPS tumors. FIG. 20C shows a circos plot demonstrating differential interaction strength and number of interactions between VP16-PPARd+ and WT as calculated using the CellChat package. FIG. 20D shows a line graph depicting relationship between species diversity per individuals for T cells between WT and VP16-PPARD conditions. FIG. 20E shows a heatmap demonstrating differential interaction strength and number of interactions between VP16-PPARD and WT as calculated using the CellChat package. FIG. 20F shows relative abundance of clonal B cells in control and vav-cre+ VP16-PPARδ+ tumor infiltrating immune cells in AKPS tumors. FIG. 20G shows a line graph depicting the relationship between species diversity per individuals for B cells between WT and VP16-PPARD conditions. The top line in the graph represents results for WT and the bottom line in the graph represents results for VP-16-PPARD. FIG. 20H shows split violin plots demonstrating the expression levels of key genes involved in B cell effector function in control and vav-cre+ VP16-PPARδ+ tumor infiltrating plasmablasts in AKPS tumors, For each gene, the expression level for diverse plasmablast is indicated on the left and the expression level for clonal plasmablast is indicated on the right. Wilcoxon rank-sum test, *** p≤0.0001

FIGS. 21A-21K include data showing the role of CXCR3 in CD8 T cells for PPARδ-induced anti-tumor effects. FIG. 21A shows a summary of differential expression of top prioritized ligands between VP16-PPARD and WT across tumor infiltrating immune cells in AKPS tumors, identified by NicheNet analysis. FIG. 21B shows a summary of ligand-target predicted interaction potential between VP16-PPARδ+ tumor infiltrating immune cells in AKPS tumors as identified by NicheNet analysis. FIG. 21C shows endpoint tumor volumes of subcutaneous MC38 tumors grown in of control and vav-cre+ VP16-PPARδ+ mice upon in vivo CXCR3+ cell depletion. n≥12, unpaired t test between VP16-PPARδ+ IgG and VP16-PPARδ+ anti-CXCR3 groups, * p≤0.05, ±SEM. FIGS. 21D-21E show Pearson Correlation Coefficient (R) and p values demonstrating the correlation between “PPAR-induced”, and (FIG. 21D) IFNg, as well as (FIG. 21E) CXCR3 z-scores in human melanoma tumor infiltrating T cells. FIG. 21F shows split violin plots demonstrating the expression levels of top predicted receptors as predicted by NicheNet between wildtype (control) and VP16-PPARδ+ tumor infiltrating CD8 T cells in AKPS tumors, Wilcoxon rank-sum test, **** p<0.0001. For each receptor, the expression level for wild-type (control) PPARδ+ tumor infiltrating CD8 T cells is indicated on the left and the expression level for VP-16 PPARδ+ tumor infiltrating CD8 T cells is indicated on the right. FIG. 21G shows a summary of ligand-target interactions between VP16-PPARδ+ tumor infiltrating immune cells in MC38 tumors, identified by NicheNet analysis. FIGS. 21H-21I shows Cxcr3 z-scores across tumor infiltrating immune cell clusters in (FIG. 21H) AKPS and (FIG. 21I) MC38 tumors, Wilcoxon rank-sum test, * p<0.05, ** p<0.01, *** p<0.001, **** p<0.0001. In FIGS. 21H-21I, for each immune cell cluster, the left hand side indicates results for control and right-hand side indicates results for Vav-cre+ VP16-PPARd+. FIG. 21J shows representative peripheral blood flow cytometry analysis demonstrating CXCR3+ CD8 T cell depletion efficiency during in vivo CXCR3+ depletion experiments, cells were gated on single cells/alive/CD3+NK1.1−/CD8+. FIG. 21K shows Pearson Correlation Coefficient (R) and p values demonstrating the correlation between “PPAR-induced” and IFNG, IRF7 and CXCR3 z-scores in human melanoma tumor infiltrating T cells.

FIGS. 22A-22D show cell intrinsic PPARδ activation is sufficient to enhance human CAR-T cell cytotoxicity. FIG. 22A shows orthotopic tumor growth curve of wild-type mice inoculated with AKPS organoids into colon sub-mucosa. Wild-type mice were sub-lethally irradiated with 5Gy total body radiation 9 days post-inoculation. 2 days later, mice were intravenously injected with 3×10⁶ in vitro stimulated wild-type CD8 T cells, or VP16-PPARδ+ CD8 T-cells and treated with 200 g anti-PD-1 antibody every 2-3 days for 14 days. Tumor index was calculated by dividing the tumor diameter by the colon diameter using colonoscopy images. Each tumor index was normalized to their respective week-1 tumor index, n≥10, unpaired t-test, * p<0.05, ±SEM. FIG. 22B shows percentage of tumor rejection at day 35 in mice that received wild-type CD8 T cells, or VP16-PPARδ CD8 T cells with anti-PD-1 after AKPS tumor inoculation, n≥10. FIG. 22C shows representative colonoscopy images at day 10 and day 35 of mice that received wild-type CD8 T cells, or VP16-PPARδ CD8 T cells with anti-PD-1 after AKPS tumor inoculation. FIG. 22D shows representative H&E images of AKPS tumors at endpoint in mice that received wild-type CD8 T cells, or VP16-PPARδ CD8 T cells with anti-PD-1 after AKPS tumor inoculation, scale bar=500 um.

### DETAILED DESCRIPTION OF CERTAIN EMBODIMENTS

Provided herein are fusion proteins that can be used to boost the activity of immune cells against cancer cells. The fusion proteins described herein comprise a peroxisome proliferator-activated receptor delta (PPAR-δ) protein sequence linked to a Herpes simplex virion protein 16 (VP16) activation domain. PPAR-δ may also be referred to as PPAR-β/δ, PPAR-β, PPARδ, PPARd, or PPARD. As demonstrated in the Examples, the VP16-PPAR-δ fusion proteins can increase immune cell-mediated cytotoxicity of tumor cells without adversely impacting normal cells from organs including lung, spleen, liver, small intestine and colon. Notably, the VP16-PPAR-δ fusion proteins may be broadly used in a variety of cancer contexts as expression of the VP16-PPAR-δ fusion protein in immune cells increased the elimination of cancer cells of different genetic backgrounds.

### Fusion Proteins

Aspects of the present disclosure provide fusion proteins comprising a PPAR-δ sequence linked to an activation domain sequence. A fusion protein is a protein comprising two heterologous proteins, protein domains, or protein fragments, that are covalently bound to each other, either directly or indirectly via linker. The linker may be a peptide linker. In some embodiments, a fusion protein is encoded by a nucleic acid comprising the coding region of a protein in frame with a coding region of an additional protein, without an intervening stop codon, thus resulting in the translation of a single protein in which the proteins are fused together. “Fuse” or “link” means to connect two different moieties and are used interchangeably. For example, two different protein sequences may be linked, e.g., via a peptide linker, to form a fusion proteins. In some embodiments, the activation domain sequence is linked to the N-terminus of the PPAR-δ sequence.

In some embodiments, a peptide linker is a poly-Glycine-Serine linker, such as a G4S linker. A G4S linker comprises the amino acid sequence Gly-Gly-Gly-Gly-Ser (SEQ ID NO: 4). In some embodiments, a G4S linker may comprise 1, 2, 3, 4, 5, 6, 7, 8, 9, 10, 15, or 20 copies of SEQ ID NO: 4. Other linkers can include Glycine-Serine (GS), a linker comprising one or more glycines (e.g., (Gly)₆ or (Gly)₈), a linker comprising A(EAAAK)₄ALEA(EAAAK)₄A (SEQ ID NO: 11), a linker comprising (GGGGS)ₙ in which n is 1, 2, 3, or 4 (SEQ ID NOs: 4, 32, 33, 34), a linker comprising (EAAAK)ₙ, in which n is 1, 2, or 3 (SEQ ID NOs: 35, 36, or 37), a linker comprising PAPAP (SEQ ID NO: 38), or a linker comprising AEAAAKEAAAKA (SEQ ID NO: 39). See also, e.g., Chen et al., Adv Drug Deliv Rev. 2013 October; 65(10):1357-69 and iGEM Parts Registry Protein domains/Linker.

### PPAR-δ

PPAR-δ is a member of the peroxisome proliferator-activated receptor (PPAR) family of transcription factors. PPAR-δ, PPAR-α, and PPAR-γ have been shown to promote expression of target genes in a ligand-dependent manner. Naturally occurring forms of PPAR-δ, PPAR-α and PPAR-γ generally comprise four functional domains: A/B, C, D, and E/F. The A/B domain is located at the N-terminus of the PPAR molecule and contains a ligand-independent activation function. This ligand-independent activation function is responsible for PPAR phosphorylation. The central C domain is conserved between PPAR subtypes. This domain comprises two zinc finger domains and allows PPAR molecules to bind to DNA at a peroxisome proliferator response element (PPRE) sequence. The D domain interacts with PPAR cofactors. The E/F domain is the ligand-binding domain and confers specificity for different ligands. To be active, wild-type PPAR molecules depend on ligand binding, heterodimerization with the RXR nuclear receptor, transcriptional cofactors, and binding to a PPRE sequence.

Despite the presence of the four functional domains in wild-type PPARs, there is relatively high structural divergence between the ligand-binding domains of the three PPARs within a given species. For example, the amino acid sequence of the ligand-binding domains of PPAR-α, PPAR-δ, and PPAR-γ within a species is only about 65% identical. See, e.g., Juge-Aubry et al., J Biol Chem. 1997 Oct. 3; 272(40):25252-9. Furthermore, the PPARs have different tissue expression profiles. PPAR-α is mainly expressed in the liver, heart, skeletal muscles, brown adipose tissue, intestine, and kidney. PPAR-γ is most highly expressed in white adipose tissue. PPAR-δ was initially speculated to be a general housekeeping gene given its near-ubiquitous tissue expression.

PPAR-δ protein sequences across species often have high sequence identity to each other. See, e.g., Table 1.

TABLE 1
Sequence Identity of PPAR-δ sequences
relative to H. sapiens PPAR-δ

Identity (%)

| Species | Gene Symbol | Protein | DNA |
|---|---|---|---|
| H. sapiens | PPARD | | |
| vs. P. troglodytes | PPARD | 99.8 | 99.3 |
| vs. M. mulatta | PPARD | 98 | 95.9 |
| vs. C. lupus | PPARD | 95.9 | 91.7 |
| vs. B. taurus | PPARD | 96.1 | 91.9 |
| vs. M. musculus | Ppard | 93.2 | 89.5 |
| vs. R. norvegicus | Ppard | 92.3 | 88.8 |
| vs. G. gallus | PPARD | 88.6 | 83 |
| vs. X. tropicalis | ppard | 71.2 | 69 |
| vs. D. rerio | pparda | 80.1 | 77.9 |
| vs. D. rerio | ppardb | 81.9 | 74.5 |

Non-limiting examples of amino acid sequences encoding PPAR-δ include UniProt KB Accession No. Q03181-1 (SEQ ID NO: 1), UniProtKB Accession No. P35396 (SEQ ID NO: 2), UniProt KB Accession No: Q03181-2 (SEQ ID NO: 14), UniProt KB Accession No: Q03181-3 (SEQ ID NO: 15), UniProt KB Accession No: Q03181-4 (SEQ ID NO: 16), and SEQ ID NO: 21. A non-limiting example of a nucleic acid sequence encoding PPAR-δ is provided in SEQ ID NO: 3, 17, and 19. See also, e.g., National Center for Biotechnology Information RefSeq Gene ID: 5467 and GenBank Accession Nos.: AY919140.1, NM_001171818.2, NM_001171819.2, NM_001171820.2, NM_006238.5, NM_177435.3, XM_005249193.2, XM_006715123.2, XM_011514707.2, XM_011514710.2, XM_017010973.2, XM_017010974.2, XM_024446474.2, XM_047418915.1, XM_047418916.1, XM_047418917.1, XM_047418918.1, XM_047418919.1, XM_047418920.1, XM_047418921.1, XM_047418922.1, XM_047418923.1, XM_047418924.1, XM_047418925.1, XM_047418926.1, XM_047418927.1, XM_047418928.1, XM_047418929.1, XM_047418930.1, XM_047418931.1, XM_047418932.1, XM_047418933.1, XM_047418934.1, XM_047418935.1, XM_047418936.1, and XM_047418937.1.

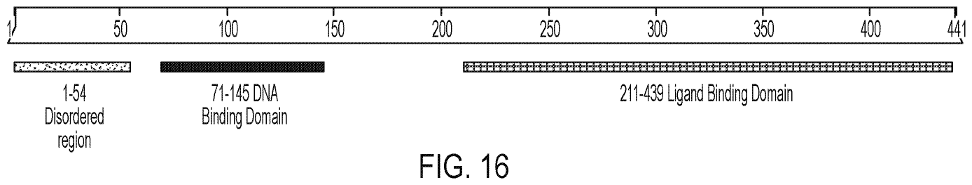

Structurally, wild-type PPAR-δ encoded by SEQ ID NO: 1 comprises a disordered region encoded by residues 1-54, a DNA binding domain encoded by residues 71-145, and a ligand binding domain encoded by residues 211-439. See, e.g., FIG. 16. Wild-type PPAR-δ encoded by SEQ ID NO: 1 includes docking sites for co-factors (e.g., cofactors involved in transcriptional regulation) and the N-terminal region of wild-type PPAR-δ encoded by SEQ ID NO: 1 can be phosphorylated to allow for ligand-independent activation of PPAR-6.

A PPAR-δ sequence for use in a fusion protein described herein may comprise or consist of a full-length PPAR-δ sequence (e.g., a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the amino acid sequence of SEQ ID NO: 1, 14, 15, or 16), or a truncated form thereof comprising or consisting of a DNA binding domain. For example, a PPAR-δ sequence may comprise or consist of one or more domains of a wild-type PPAR-δ sequence. In some embodiments, a PPAR-δ sequence disclosed herein comprises or consists of a DNA binding domain. In some embodiments, a PPAR-δ sequence comprises or consists of a DNA binding domain corresponding to positions 71-145 of SEQ ID NO: 1. In some embodiments, a PPAR-δ sequence comprises or consists of a DNA binding domain and a ligand binding domain. In some embodiments, a PPAR-δ sequence comprises or consists of a DNA binding domain and a ligand binding domain corresponding to positions 211-439 of SEQ ID NO: 1. In some embodiments, a PPAR-δ sequence comprises or consists of a DNA binding domain and a disordered region (e.g., at the N-terminus). In some embodiments, a PPAR-δ sequence comprises or consists of a DNA binding domain and a disordered region corresponding to positions 1-54 of SEQ ID NO: 1. In some embodiments, a PPAR-δ sequence comprises or consists of a DNA binding domain and (i) a ligand domain and/or (ii) a disordered region. In some embodiments, a PPAR-δ sequence comprises or consists of a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the amino acid sequence of SEQ ID NO: 24 and a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the amino acid sequence of SEQ ID NO: 25, and/or a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the amino acid sequence of SEQ ID NO: 26.

In some embodiments, a PPAR-δ sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the amino acid sequence of SEQ ID NO: 1, or SEQ ID NOs: 14-16.

In some embodiments, a PPAR-δ comprises a sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the amino acid sequence of SEQ ID NO: 21 or SEQ ID NOs: 24-26.

In some embodiments, a nucleotide sequence encoding a PPAR-δ sequence comprises a nucleic acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to the nucleic acid sequence of SEQ ID NO: 3, 17, 19, or a PPAR-δ sequence disclosed herein.

The term “identity” refers to the overall relatedness between biological molecule, for example, polypeptide molecules or nucleic acid molecules. Calculation of the percent identity of two sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The residues (e.g., amino acid or nucleic acid) at corresponding positions are then compared. When a residue at a position in the first sequence is occupied by the same residue as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. Exemplary computer software to determine homology between two sequences include, but are not limited to BLASTP, BLASTN, CLUSTAL, and MAFFT, using, e.g. default parameters. In some embodiments, a sequence is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to an amino acid or nucleic acid disclosed herein.

In some embodiments, a PPAR-δ sequence is not a wild-type PPAR-δ sequence and retains at least 25% to 100% (e.g., at least 25%, at least 50%, at least 75%, or 100%, including all values in between) of the activity of a wild-type PPAR-δ sequence. Non-limiting examples of PPAR-δ activity include the ability of a PPAR-δ sequence to bind to a PPRE in the promoter of a PPAR-δ target gene and the ability of a PPAR-δ sequence to drive expression of a PPAR-δ target gene.

### Activation Domains

An “activation domain,” as used herein, refers to a protein or protein domain that in conjunction with a DNA binding domain (e.g., a DNA binding domain from a PPAR-δ), can activate transcription from a promoter. Any activation domains known in the art may be used in accordance with the present disclosure.

Non-limiting examples of activation domains include: a Herpes simplex virion protein 16 (VP16) activation domain, VP64 activation domain, VP48 activation domain, VP160 activation domain, MYOD activation domain, and FOXA activation domain. In some embodiments, an activation domain is a transcriptional activator domain selected from HSF1, VP16, VP64, p65, RTA, MyoD1, SET7, VPR, histone acetyltransferase p300, TET1 hydroxylase catalytic domain, LSD1, CIB1, AD2, CR3, GATA4, p53, SP1, MEF2C, TAX, PPAR-gamma, and SET9. See also, e.g., US 20190351074.

As used herein, a “VP16 activation domain” comprises the amino acid sequence DALDDFDLDML (SEQ ID NO: 6). In Herpes simplex virus, the VP16 protein comprises SEQ ID NO: 6 in the transactivation domain (TAD). SEQ ID NO: 6, for example, is located at residues 437 to 447 of SEQ ID NO: 23. A VP16 activation domain sequence described herein may further comprise additional segments from VP16. As non-limiting examples, a VP16 activation domain sequence may comprise amino acids 411-490, 410-452 or amino acids 453-490 from the wild-type VP16 protein sequence (e.g., SEQ ID NO: 23). In some embodiments, a VP16 activation domain sequence comprises a residue corresponding to position 442 in SEQ ID NO: 23. See also, e.g., Hirai et al, Int J Dev Biol. 2010; 54(11-12): 1589-1596. VP64 activation domains comprise four copies of SEQ ID NO: 6 in which the copies are linked by a GS linker. VP64 activation domains comprise SEQ ID NO: 7.

In some embodiments, an activation domain sequence comprises one to ten copies (e.g., one, two, three, four, five, six, seven, eight, nine, or ten copies) of SEQ ID NO: 6. In some embodiments, the copies of SEQ ID NO: 6 are linked by one or more amino acids. In some embodiments, an activation domain sequence comprises or consists of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to SEQ ID NO: 6 or SEQ ID NO: 7.

As used herein, a “MYOD activation domain” sequence is derived from the transactivation domain of MyoD. For example, a MyoD activation domain sequence may comprise residues 3 to 56 of MyoD or a truncated form thereof. See, e.g., Hirai et al, Int J Dev Biol. 2010; 54(11-12): 1589-1596 and Bergstrom and Tapscott Mol Cell Biol. 2001; 21:2404-2412.

As used herein, a “FOXA activation domain” sequence is derived from the transactivation domain of FOXA. For example, a FOXA activation domain sequence may comprise residues 14 to 93 or a truncated form there of and/or residues 361 to 458 of FOXA or a truncated form thereof. See, e.g., Hirai et al, Int J Dev Biol. 2010; 54(11-12): 1589-1596 and Qian and Costa Nucleic Acids Res. 1995; 23:1184-1191.

In some embodiments, a VP16-PPAR-δ sequence comprises or consists of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 31.

In some embodiments, a nucleotide sequence encoding a VP16-PPAR-δ sequence comprises or consists of a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 30.

### CPT1A

CPT1A is an isoform of carnitine palmitoyltransferase 1 that is expressed in liver, kidney, brain, pancreas, leukocytes, fibroblasts, and embryonic tissues. CPT1A catalyzes mitochondrial fatty acid oxidation. Structurally, wild-type CPT1A generally comprises an N-terminal regulatory domain and a C-terminal catalytic domain that is separated by two transmembrane helices. See, e.g., Samanta et al., Biopolymers. 2014 April; 101(4): 398-405.

“CPT1A” as used herein encompasses wild-type and CPT1A sequences comprising one or more mutations relative to a wild-type sequence. In some embodiments, a CPT1A disclosed herein comprises histidine (H) at a residue corresponding to position 473 in SEQ ID NO: 12. Without being bound by a particular theory, the residue corresponding to position 473 may be involved in binding L-carnitine, which is a co-factor for CPT1A that is involved in transporting long-chain acyl-COA from the cytosol to the mitochondria.

Wild-type CPT1A is inhibited by malonyl-COA. However, CPT1A may be mutated to render the enzyme insensitive to malonyl-COA inhibition. In some embodiments, a CPT1A disclosed herein is constitutively active. As a non-limiting example, rat CPT1AM encoded by SEQ ID NO: 13 is a constitutively active form of rat CPT1A. See also, e.g., Morillas et al., J Biol Chem. 2003 Mar. 14; 278(11):9058-63. In some embodiments, a CPT1A mutation decreases inhibition of CPT1A activity by malonyl-COA by at least 5% to 100%, e.g., at least 5%, at least 10%, at least 25%, at least 50%, at least 75%, or 100%. In some embodiments, a CPT1A disclosed herein comprises a mutation at a residue corresponding to position 593 of SEQ ID NO: 12. For example, a CPT1A disclosed herein may comprise a M593S, M593A, or a M593E mutation relative to SEQ ID NO: 12, which renders the CPT1A less sensitive to malonyl-coA inhibition.

In some embodiments, a nucleotide sequence encoding CPT1A comprises or consists of a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to SEQ ID NO: 27. In some embodiments, an amino acid sequence encoding CPT1A is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to SEQ ID NO: 12.

CPT1A activity may be determined using any suitable method. For example, CPT1A activity may be determined by the level of carnitine in a cell. An increase in CPT1A activity may be determined as a decrease in the level of carnitine.

### Methods of Engineering Immune Cells

Aspects of the present disclosure provide a method of engineering immune cells by inducing PPAR-δ signaling a VP16-PPAR-δ fusion and/or increasing CPT1A activity in immune cells. Any suitable method may be used to introduce a VP16-PPAR-δ fusion protein disclosed herein into an immune cell and/or to increase activity of CPT1A. For example, a VP16-PPAR-δ fusion protein may be introduced into an immune cell by introducing into the cell a nucleic acid encoding the fusion protein. Alternatively, a VP16-PPAR-δ fusion protein may be introduced into an immune cell by introducing the protein into the cell. In some embodiments, CPT1A activity is increased in an immune cell using a nucleic acid or an amino acid sequence encoding CPT1A. The CPT1A may be a wild-type CPT1A sequence or comprise one or more amino acid substitutions relative to a wild-type CPT1A. For example, CPT1A activity may be increased by increasing expression of wild-type CPT1A and/or by increasing expression of a CPT1A comprising one or more amino acid substitutions, deletions, and/or insertions relative to a wild-type CPT1A. In some embodiments, CPT1A expression is increased by introducing an engineered nucleic acid encoding wild-type CPT1A, introducing an engineered protein encoding wild-type CPT1A, and/or increasing expression of an endogenous CPT1A gene (e.g., using gene-editing technologies including Clustered Regularly Interspaced Short Palindromic Repeats (CRISPR) activation). In some embodiments, a constitutively active CPT1A is introduced to an immune cell (e.g., using an engineered nucleic acid, an engineered protein, and/or gene editing).

### Engineered Nucleic Acids and Proteins

A “nucleic acid” is at least two nucleotides covalently linked together. In some instances, a nucleic acid comprises one or more phosphodiester bonds (e.g., a phosphodiester “backbone”). In some embodiments, a nucleic acid is an engineered polynucleotide. An engineered polynucleotide is a nucleic acid that does not occur in nature. Engineered polynucleotides include recombinant nucleic acids and synthetic nucleic acids. A recombinant nucleic acid is a molecule that is constructed by joining nucleic acids from two different sources (e.g., joining of a human sequence with a mouse sequence or joining of the exons of gene to produce a coding sequence) by joining two or more nucleic acids in a non-natural configuration, or by altering the sequence of a nucleic acid. A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered polynucleotide may comprise DNA (e.g., genomic DNA, cDNA or a combination of genomic DNA and cDNA), RNA or be a hybrid molecule. As a non-limiting example, an engineered polynucleotide may comprise any combination of deoxyribonucleotides and/or ribonucleotides (e.g., artificial or natural), including any combination of two or more bases, including uracil, adenine, thymine, cytosine, guanine, inosine, xanthine, hypoxanthine, isocytosine and/or isoguanine.

Engineered polynucleotides of the present disclosure may be produced using standard molecular biology methods (see, e.g., Green and Sambrook, Molecular Cloning, A Laboratory Manual, 2012, Cold Spring Harbor Press).

In some embodiments, an engineered polynucleotide comprises a promoter operably linked to one or more nucleic acid sequences. A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be operably linked when it is in a correct functional location and orientation in relation to a nucleic acid sequence it regulates to control (“drive”) transcriptional initiation and/or expression of that sequence.

A promoter may be one naturally associated with a gene or sequence. Such a promoter can be referred to as “endogenous.” For example, a VP16-PPAR-δ fusion protein disclosed herein may bind to the endogenous promoter of one or more target genes.

In some embodiments, a coding nucleic acid sequence is positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR). For example, a nucleic acid sequence encoding a VP16-PPAR-δ fusion protein may comprise a heterologous promoter that drives expression of the fusion protein. In some embodiments, a nucleic acid sequence encoding CPT1A comprises a heterologous promoter that drives expression of CPT1A.

In some embodiments, a promoter is a constitutive promoter. In some embodiments, a constitutive promoter is a cell-specific promoter. In some embodiments, a promoter is a tissue-specific promoter. In some embodiments, a promoter described herein drives expression of an operably linked gene in an immune cell. For example, a promoter may drive expression in a myeloid cell or a lymphoid cell. In some embodiments, a promoter is a VAV promoter. See, e.g., de Boer et al. Eur. J. Immunol. 2003. In some embodiments, a promoter is a LysMcre promoter.

In some embodiments, the promoter sequence comprises a mammalian promoter. In some embodiments, the promoter sequence is a SV40 promoter, a CMV promoter, a UBC promoter, an EF1A promoter, a PGK promoter, or a CAG promoter.

In some embodiments, a promoter is an inducible promoter. An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, an engineered nucleic acid disclosed herein comprises an expression cassette with a transcriptional start site. In some embodiments, the expression cassette further comprises a transcriptional terminator between the transcriptional start site and an open reading frame. In some embodiments, the transcriptional terminator is removable. In some embodiments, the transcriptional terminator is removable using a Cre-Lox system. For example, the transcriptional terminator may be flanked by lox sites that is removed upon introduction of Cre recombinase. In some embodiments, the expression of Cre recombinase is controlled by a tissue-specific promoter.

In some embodiments, an engineered nucleic acid disclosed herein comprises an expression cassette that comprises a translational start site. In some embodiments, the expression cassette further comprises a stop codon after the translational start site and before a protein coding region. In some embodiments, the stop codon is removable. In some embodiments, the stop codon is removable using a Cre-Lox system. In some embodiments, the stop codon is flanked by lox sites and Cre recombinase is encoded elsewhere in the genome.

In some embodiments, an engineered nucleic acid is present in a vector (e.g., an expression vector). A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is a viral vector. Non-limiting examples of viral vectors include retroviral vectors, lentiviral vectors, adenoviral vectors, and adeno-associated viral vectors, poxvirus vectors, alpha virus vectors, baculovirus vectors, or vesicular stomatitis virus vectors.

In some embodiments, a VP16-PPAR-δ fusion protein and/or CPT1A protein is delivered directly into a cell. Suitable methods for protein delivery are known in the art. Non-limiting examples of protein delivery include electroporation, microinjection, use of cell-penetrating peptides, use of protein transduction domains, use of liposomes, and use of nanoparticles.

Any suitable method may be used to determine the presence of PPAR-δ activity in immune cells comprising a VP16-PPAR-δ fusion protein. For example, the expression of one or more PPAR-δ target genes may be detected. For example, mRNA and/or protein levels of one or more target genes may be detected before and after introduction of a VP16-PPAR-δ fusion protein. In some embodiments, the expression of ACADVL, ACAA2, ANGPTL4, CAT, CPT1A, FABP4, ECH1, PDK4, SLC25A20 and/or PLIN2 is detected. In some embodiments, introduction of a VP16-PPAR-δ fusion protein increases expression of a PPAR-δ target gene by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) as compared to a control. In some embodiments, a control is the level of expression of the PPAR-δ target gene without the introduction of the VP16-PPAR-δ fusion protein.

In some embodiments, the methods of engineering immune cells disclosed herein specifically increase PPAR-δ activity and do not comprise increasing the activity of PPAR-α, PPAR-γ, or a combination thereof. In some embodiments, the activity of a particular PPAR subtype refers to activity that is unique to a PPAR relative to another PPAR subtype. For example, PPAR-α activity may increase expression of SLC27A1, NR1H3, FABP1, ALDH9A1, RETSAT, IL1RN, PEX11A, CREB3L3, MAP3K8, SLC22A1, C3, GRHPR, ACOT1, ARNTL, NFKBIA, FATP1, CYP4A6, IGF1, LPL, PLA2G2A, CD36, ACOX1, HMGCS2, DBI, CYP4A1, EHHADH, PDX1, ACAA1B, SEMA6B, SCARBI, SCP2, CYP8B1, CYP4F1, MEl, HILPDA, ALDH3A2, AKR1C18, SCD1, PLTP, SLC10A2, HADH, CYP2C8, VNN1, SLC25A1, LRP2, CIDEA, GPD1, LIPA, CYP3A4, UGT2B4, UGT1A9, CPT1B, AP2A2, SULT2A1, NR1D1, ABCG2, TXN, ACADM, APOA5, CYP1A1, PDZK1, FADS2, GPT, AHR, APOA1, and/or SLC29A1. In some embodiments, PPAR-δ activity does not increase expression of SLC27A1, NR1H3, FABP1, ALDH9A1, RETSAT, IL1RN, PEX11A, CREB3L3, MAP3K8, SLC22A1, C3, GRHPR, ACOT1, ARNTL, NFKBIA, FATP1, CYP4A6, IGF1, LPL, PLA2G2A, CD36, ACOX1, HMGCS2, DBI, CYP4A1, EHHADH, PDX1, ACAA1B, SEMA6B, SCARBI, SCP2, CYP8B1, CYP4F1, MEl, HILPDA, ALDH3A2, AKR1C18, SCD1, PLTP, SLC10A2, HADH, CYP2C8, VNN1, SLC25A1, LRP2, CIDEA, GPD1, LIPA, CYP3A4, UGT2B4, UGT1A9, CPT1B, AP2A2, SULT2A1, NR1D1, ABCG2, TXN, ACADM, APOA5, CYP1A1, PDZK1, FADS2, GPT, AHR, APOA1, and/or SLC29A1. In some embodiments, PPAR-γ activity increases expression of ASS1, DBI, TSC22D1, GPD1, GOS2, HCAR1, NAMPT, INSR, TFF2, PGK1, PKM, APOE, PTGS2, LRP1, SCARBI, SGK1, BRCA1, SAT1, BCL2, CAV1, CYP27A1, GHITM, TXNIP, SDC1, REN, SHBG, GCK, SLC9A1, CAT, APOA1, SLC22A5, CTSL1, TNFSF10, TNIP1, KLF4, IRF1, UGT1A9, PCK1, AQP7, BCMO1, SCNN1G, OLR1, CIDEA, CIDEC, TUSC5, PLA2G16, ARNTL, RBP7, GPR81, AACS, ADCY6, RARRES2, PFKFB3, RHOBTB1, KL, HP, TMEM143, 1100001G20RIK, FGF1, UCP1, LPL, SLC27A1, SORBS1, UCP2, MC2R, GSTA2, PCX, BACE1, SERPINE1, LIPE, PDX1, VLDLR, GIPR, LRP2, CD36, FABP4, MUC1, RGS5, SLC2A2, FATP1, SLC1A2, NR1D1, PLA2G2A, SLC25A1, HMGCS2, RBP4, MRAP, and/or ADIPOQ. In some embodiments, PPAR-δ activity does not increase expression of expression of ASS1, DBI, TSC22D1, GPD1, GOS2, HCAR1, NAMPT, INSR, TFF2, PGK1, PKM, APOE, PTGS2, LRP1, SCARBI, SGK1, BRCA1, SAT1, BCL2, CAV1, CYP27A1, GHITM, TXNIP, SDC1, REN, SHBG, GCK, SLC9A1, CAT, APOA1, SLC22A5, CTSL1, TNFSF10, TNIP1, KLF4, IRF1, UGT1A9, PCK1, AQP7, BCMO1, SCNN1G, OLR1, CIDEA, CIDEC, TUSC5, PLA2G16, ARNTL, RBP7, GPR81, AACS, ADCY6, RARRES2, PFKFB3, RHOBTB1, KL, HP, TMEM143, 1100001G20RIK, FGF1, UCP1, LPL, SLC27A1, SORBS1, UCP2, MC2R, GSTA2, PCX, BACE1, SERPINE1, LIPE, PDX1, VLDLR, GIPR, LRP2, CD36, FABP4, MUC1, RGS5, SLC2A2, FATP1, SLC1A2, NR1D1, PLA2G2A, SLC25A1, HMGCS2, RBP4, MRAP, and/or ADIPOQ. See also, e.g., Fang et al. PPAR Res. 2016; 2016:6042162.

### Immune Cells

A VP16-PPAR-δ fusion protein and/or CPT1A disclosed herein may be introduced into any type of immune cell. An immune cell may be characterized by its lineage or the precursor from which it is derived. For instance, an immune cell may be a lymphoid cell or a myeloid cell. Non-limiting examples of lymphoid cells include natural killer cells, T cells, and B cells. Non-limiting examples of myeloid cells include granulocytes, monocytes, macrophages, and dendritic cells. In some embodiments, the immune cells are neutrophils. In some instances, a myeloid cell expresses CXCL10. In some embodiments, the immune cells are dendritic cells. In some embodiments, the immune cells are monocytes. In some embodiments, the immune cells are myeloid-derived suppressor cells (MDSC). In some embodiments, the immune cells are macrophages. In some embodiments, the immune cells are M1 macrophages. In some embodiments, the immune cells are M2 macrophages.

In some embodiments, an immune cell is an immune stem cell. In some embodiments, an immune stem cell is a bone marrow stem cell, an hematopoietic stem cell, a common lymphocyte progenitor, or a common myeloid progenitor.

An immune cell may be characterized by whether the immune cell is proinflammatory. A proinflammatory immune cell activates the immune system. Non-limiting examples of proinflammatory immune cells include CD8+ T cells and natural killer cells. In contrast a tolerogenic immune cell has immunosuppressive activity. Non-limiting examples of tolerogenic immune cells include M2-like macrophages, myeloid derived suppressive cells (MDSCs) and regulatory T cells (Treg). Without being bound by a particular theory, the VP16-PPAR-δ fusions and CPT1A disclosed herein may be useful in increasing expression of proinflammatory genes in proinflammatory immune cells and in immunosuppressive cells.

In some instances, an immune cell disclosed herein comprises one or more polynucleotides encoding one or more markers and/or PPAR-δ target genes. For example, an immune cell disclosed herein may comprise a polynucleotide encoding CXCR3, CD3, CD4, CD8, CD25, ACADVL, ACAA2, ANGPTL4, CAT, CPT1A, FABP4, ECH1, PDK4, SLC25A20 and/or PLIN2.

T cells or T lymphocytes are a type of white blood cell and play an important role in adaptive immunity. There are two major types of T cells: helper T cells and the cytotoxic T cells. Helper T cells assist other cells of the immune system carry out their functions, while cytotoxic T cells mediate killing of cells, including the killing of infected cells and tumor cells. In some instances, a T cell is characterized by the presence of one or more markers, including one or more cell surface markers. Non-limiting examples of markers include CXCR3, CD3, CD4, CD8, and CD25. In some embodiments a T cell is a CD8+ T cell, a gamma delta T lymphocyte, a regulatory T cells (Treg), a proliferating regulatory T cell, a natural killer T cell (NKT), a CD4+ T cell, a Th17 cell, or a Th2 cell.

T cells may be engineered to express a chimeric antigen receptor (CAR). For example, T cells may be engineered with a CAR that recognizes a specific tumor antigen, which may be useful for localizing the T cells to a cancer cell to kill the cancer cell. CARs typically comprise an antigen-binding domain (or extracellular targeting domain), a transmembrane domain, and an intracellular signaling domain. In some embodiments, the CAR further comprises at least one co-stimulatory intracellular domain.

The antigen-binding domain of a CAR may comprise an antibody. The term “antibody” includes full-length antibodies and any antigen binding fragment or single chain thereof. The term “antibody” includes, without limitation, a glycoprotein comprising at least two heavy (H) chains and two light (L) chains inter-connected by disulfide bonds, or an antigen binding portion thereof. Antibodies may be polyclonal or monoclonal; xenogeneic, allogeneic, or syngeneic; or modified forms thereof (e.g., humanized, chimeric).

The term “antigen-binding fragment” of an antibody refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include (i) a Fab fragment, a monovalent fragment consisting of the VH, VL, CL and CHi domains; (ii) a F(ab′)2 fragment, a bivalent fragment comprising two Fab fragments linked by a disulfide bridge at the hinge region; (iii) a Fd fragment consisting of the VH and CHi domains; (iv) a Fv fragment consisting of the VH and VL domains of a single arm of an antibody, (v) a dAb fragment (Ward et al., Nature 341:544 546, 1989), which consists of a VH domain; and (vi) an isolated complementarity determining region (CDR) or (vii) a combination of two or more isolated CDRs, which may optionally be joined by a synthetic linker. Furthermore, although the two domains of the Fv fragment, VH and VL, are coded for by separate genes, they can be joined, using recombinant methods, by a synthetic linker that enables them to be made as a single protein chain in which the VH and VL regions pair to form monovalent molecules (known as single chain Fv (scFv); see, e.g., Bird et al. Science 242:423 426, 1988; and Huston et al. Proc. Natl. Acad. Sci. USA 85:5879-5883, 1988). Such single chain antibodies are also encompassed within the term “antigen-binding portion” of an antibody. These antibody fragments may be obtained using conventional techniques known to those with skill in the art, and the fragments are screened for utility in the same manner as are intact antibodies.

Non-limiting examples of antibodies and fragments thereof include: bevacizumab (AVASTIN®), trastuzumab (HERCEPTIN®), alemtuzumab (CAMPATH®, indicated for B cell chronic lymphocytic leukemia,), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-C5, IOR-T6 (anti-CD1), IOR EGF/R3, celogovab (ONCOSCINT® OV103), epratuzumab (LYMPHOCIDE®), pemtumomab (THERAGYN®), Gliomab-H (indicated for brain cancer, melanoma).

### Increasing CPT1A Activity

Aspects of the present disclosure provide methods of engineering immune cells comprising increasing CPT1A activity. As used herein, “increasing CPT1A activity” refers to activation of CPT1A. In some embodiments, CPT1A is activated by increasing expression of a CPT1A disclosed herein. In some embodiments, an effective amount of a CPT1A disclosed herein is introduced into an immune cell to increase expression of a CPT1A by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the CPT1A expression in the immune cell in the absence of introducing the CPT1A. In some embodiments, the CPT1A protein is introduced directly into a cell. In some embodiments, a nucleic acid encoding CPT1A is introduced into a cell. In some embodiments, the nucleic acid is on an expression vector.

In some embodiments, a CPT1A comprises one or more amino acid substitutions, deletions, and/or insertions that increases the activity of the CPT1A relative to a wild-type CPT1A. For example, one or more amino acid substitutions, deletions, and/or insertions may increase CPT1A activity by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is a wild-type CPT1A.

### Applications

Aspects of the present disclosure provide methods comprising introducing any of the VP16-PPAR-δ fusion proteins and/or CPT1A disclosed herein into an immune cell, which may be useful in increasing the anti-tumor immunity of the immune cell. In some instances, a method described herein may comprise introducing or administering an effective amount of any of the engineered nucleic acids, engineered proteins, or compositions comprising the same to an immune cell. The immune cell may be in vitro (e.g., cultured cell), ex vivo (e.g., isolated from a subject), or in vivo (e.g., in a subject).

An “effective amount” refers to an amount sufficient to elicit the desired biological response. Non-limiting examples of desired biological responses include increasing or decreasing gene expression and treating a condition. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. In some embodiments, any of the engineered nucleic acids, any of the engineered proteins, any of the compositions disclosed herein is administered in an effective amount. In some embodiments, any of the immune cells disclosed herein is administered in a number sufficient to elicit the desired biological response.

In some embodiments, an effective amount of a VP16-PPAR-δ fusion protein is the amount sufficient to increase the expression of a PPAR-δ target gene. In some embodiments, the amount is sufficient to increase the expression of a PPAR-δ target gene by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the level of gene expression in the immune cell in the absence of the VP16-PPAR-δ fusion protein. In some embodiments, the PPAR-δ target gene is CPT1A.

In some embodiments, an effective amount of a CPT1A is the amount sufficient to increase CPT1A activity in an immune cell. In some embodiments, the amount is sufficient to increase the CPT1A activity by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the level of gene expression in the immune cell in the absence of a CPT1A that is introduced.

### Method of Increasing the Anti-Tumor Activity of Immune Cell

Cancer immunotherapy is dependent upon the effector functions of immune cells, including immune cell-mediated cytotoxicity of tumor cells. Studies have shown that the activity of immune cells can wane over time resulting in exhaustion, while other studies have shown that immune cell-mediated cytotoxicity may be dependent on the genetic background of a target cell. Immune cell exhaustion may refer to T cell exhaustion in which T cells have a reduced ability to secrete cytokines and show increased expression of inhibitory receptors as compared to a control, e.g., a control may be a T cell that has not been activated. T-cell receptors are protein complexes that activate T cells by recognizing antigens on the surface of T cells and inducing a signaling cascade within T cells in response. In some embodiments, by increasing the anti-tumor activity of immune cells in a target cell-independent manner, the VP16-PPAR-δ fusion proteins disclosed herein may be used to address many of these limitations.

In some embodiments, a VP16-PPAR-δ fusion protein is introduced into an immune cell and/or CPT1A activity is increased in an immune cell in an amount that is sufficient to increase infiltration of the immune cell into a tumor. In some instances, immune cell infiltration is determined as the number or percentage of immune cells within a tumor. In some embodiments, immune cell infiltration is determined as the ratio of immune cells to tumor cells within a tumor. Any suitable method of determining immune cell infiltration may be used including RNA sequencing and cell staining to distinguish between immune cells and tumor cells.

In some embodiments, a VP16-PPAR-δ fusion protein is introduced into an immune cell and/or CPT1A activity is increased in an immune cell in an amount that is sufficient to increase the lifespan of an immune cell. For example, the amount is sufficient to increase the lifespan of an immune cell by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the lifespan of the immune cell in the absence of the VP16-PPAR-δ fusion protein and/or increase in CPT1A activity. Lifespan may be determined using any suitable method including the length of time a cell is viable. For example, the half-life of a cell may be determined. Cell viability may be determined by the presence or absence of one or more cell markers, including the level of ATP in the cell, the ability of a cell to reduce a substrate and/or detecting the activity of one or more enzymes in the cells.

In some instances, a VP16-PPAR-δ fusion protein is introduced into an immune cell and/or CPT1A activity is increased in an immune cell in an amount that is sufficient to increase expression of one or more pro-inflammatory molecules. For example, the amount is sufficient to increase the expression of one or more pro-inflammatory molecules by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the level of expression of the one or more proinflammatory molecules in the immune cell in the absence of the VP16-PPAR-δ fusion protein and/or increase in CPT1A activity. In some embodiments, a proinflammatory molecule is a molecule whose expression is upregulated by an interferon. See also, e.g., Liberzon et al., 2015 Cell Syst 1, 417-425. In some embodiments, a proinflammatory molecule is a proinflammatory cytokine. Non-limiting examples of proinflammatory molecules include ISG15, IRF7, IRF1, IFIT3, IFI208, CXCL10, and TNF-α.

In some instances, a VP16-PPAR-δ fusion protein is introduced into an immune cell and/or CPT1A activity is increased in an immune cell in an amount that is sufficient to increase expression of one or more cell migration genes, T cell effector function genes, histone proteins, and/or survival genes. For example, the amount is sufficient to increase the expression of one or more migration genes, T cell effector function genes, histone proteins, and/or survival genes by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the level of expression of the one or more migration genes, T cell effector function genes, histone proteins, and/or survival genes in the immune cell in the absence of the VP16-PPAR-δ fusion protein and/or increase in CPT1A activity. In some embodiments, a migration gene is CXCR3, CXCR1, and/or CCR6, an effector function gene is GZMK, GZMB, GZMF, IFNG, and/or TBX21, a histone protein is HIST4H4 and/or HIST1H4M, and/or a survival gene is BCL2, FOS, and/or JUN).

In some instances, a VP16-PPAR-δ fusion protein is introduced into an immune cell and/or CPT1A activity is increased in an immune cell in an amount that is sufficient to decrease expression of one or more T cell dysfunction markers and/or T_{reg} destabilization markers. For example, the amount is sufficient to decrease the expression of one or more T cell dysfunction markers and/or T_{reg} destabilization markers by at least 25% to 1,000% (e.g., at least 25%, at least 50%, at least 75%, or up to 100%, including all values in between) compared to a control. In some embodiments, the control is the level of expression of the one or more T cell dysfunction markers and/or T_{reg} destabilization markers in the immune cell in the absence of the VP16-PPAR-δ fusion protein and/or increase in CPT1A activity. Non-limiting examples of T cell dysfunction markers include PD-1 (e.g., encoded by PDCD1), Tim-3 (e.g., encoded by HAVCR2) and LAG-3. In some embodiments, a T_{reg} destabilization marker is FOXP3.

### Methods of Treatment

Aspects of the present disclosure provide methods of treating cancer comprising administering to a subject in need thereof: a sufficient number of immune cells comprising a VP16-PPAR-δ fusion protein to treat the cancer and/or a sufficient number of immune cells having increased CPT1A activity to treat the cancer. A composition comprising an immune cell disclosed herein may further comprise additional agents (e.g. for specific delivery, increasing half-life, or other therapeutic agents). In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In administering immune cells to a subject, a “pharmaceutically acceptable carrier” is a pharmaceutically acceptable material, composition or vehicle that is compatible with maintaining the viability of the cells.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or based on diagnostic parameters). Treatment may also be continued after symptoms have resolved, for example, to delay or prevent recurrence. A sufficient number of any of the immune cells comprising an engineered nucleic acid and/or engineered protein disclosed herein may be administered to a subject in need thereof to treat the subject. In some embodiments, a sufficient number of immune stem cells comprising an engineered nucleic acid and/or engineered protein disclosed herein may be administered to a subject in need thereof to treat the subject.

For the purpose of the present disclosure, the effective number of immune cells comprising an engineered nucleic acid and/or engineered protein as described herein will depend on the specific agent (or compositions thereof) employed, the formulation and route of administration, the type and severity of the disorder, previous therapy, the subject's clinical history and response to the agents, and the discretion of the attending physician. Typically, the clinician will administer an agent until a dosage is reached that achieves the desired result. Administration can be continuous or intermittent, depending, for example, upon the recipient's physiological condition, and other factors known to skilled practitioners. The administration of an agent may be essentially continuous over a preselected period of time or may be in a series of spaced dose, e.g., either before, during, or after developing a disorder.

A “subject” refers to humans and non-human animals, such as apes, monkeys, horses, cattle, sheep, goats, dogs, cats, rabbits, guinea pigs, rats, and mice. In one embodiment, the subject is a human. In some embodiments, the subject is an experimental animal or organoids as a disease model. A “subject in need thereof” refers to a subject who has or is at risk of a disease or disorder (e.g., cancer).

Immune cells comprising an engineered nucleic acid and/or engineered protein of the present disclosure may be delivered to a subject (e.g., a mammalian subject, such as a human subject) by any in vivo delivery method known in the art. For example, such cells may be delivered into a subject intravenously. In some embodiments, immune cells comprising an engineered nucleic acid and/or engineered protein disclosed herein are delivered systemically to a subject having a cancer or other disease and produces a therapeutic molecule specifically in cancer cells or diseased cells of the subject. In some embodiments, immune cells comprising an engineered nucleic acid and/or engineered protein disclosed herein are delivered to a site of the disease or disorder (e.g., site of cancer).

Non-limiting examples of cancers that may be treated using the immune cells comprising an engineered nucleic acid and/or engineered protein disclosed herein and methods described herein include: premalignant neoplasms, malignant tumors, metastases, or any disease or disorder characterized by uncontrolled cell growth such that it would be considered cancerous or precancerous. The cancer may be a primary or metastatic cancer. Cancers include, but are not limited to, ocular cancer, biliary tract cancer, bladder cancer, pleura cancer, stomach cancer, ovary cancer, meninges cancer, kidney cancer, brain cancer including glioblastomas and medulloblastomas, breast cancer, cervical cancer, choriocarcinoma, colon cancer, endometrial cancer, esophageal cancer, gastric cancer, hematological neoplasms including acute lymphocytic and myelogenous leukemia, multiple myeloma, AIDS-associated leukemias and adult T-cell leukemia lymphoma, intraepithelial neoplasms including Bowen's disease and Paget's disease, liver cancer, lung cancer, lymphomas including Hodgkin's disease and lymphocytic lymphomas, neuroblastomas, oral cancer including squamous cell carcinoma, ovarian cancer including those arising from epithelial cells, stromal cells, germ cells and mesenchymal cells, pancreatic cancer, prostate cancer, rectal cancer, sarcomas including leiomyosarcoma, rhabdomyosarcoma, liposarcoma, fibrosarcoma, and osteosarcoma, skin cancer including melanoma, Kaposi's sarcoma, basocellular cancer, and squamous cell cancer, testicular cancer including germinal tumors such as seminoma, non-seminoma, teratomas, choriocarcinomas, stromal tumors and germ cell tumors, thyroid cancer including thyroid adenocarcinoma and medullar carcinoma, and renal cancer including adenocarcinoma and Wilms' tumor. Commonly encountered cancers include breast, prostate, lung, ovarian, colorectal, and brain cancer. In some embodiments, the cancer is a melanoma, carcinoma, sarcoma, or lymphoma. In some embodiments, a cancer is a non-hematological cancer.

A cancer cell may be characterized by the presence of one or more tumor antigens. A “tumor antigen” is a protein or other molecule that is found on a cancer cell. In some embodiments, a tumor antigen is a protein or molecule that is found on a cancer cell and not on a normal (non-cancerous) cell. In some embodiments, a tumor antigen is a protein or molecule that has increased expression relative to a normal cell. In some embodiments, a tumor antigen is a receptor tyrosine kinase. For example, a receptor tyrosine kinase may be a member of the ErbB family of receptors, which include epidermal growth factor receptor (EGFR), ERBB2 (HER2), ERBB3 (HER3), and ERBB4 (HER4).

In some embodiments, immune cells comprising an engineered nucleic acid and/or engineered protein disclosed herein is administered to a subject in a number sufficient to increase the number of tumor-infiltrating immune cells in a tumor of the subject compared to a control. In some embodiments, the control is the number of tumor-infiltrating immune cells in the tumor without administering the immune cell comprising the engineered nucleic acid and/or engineered protein disclosed herein.

In some embodiments, any of the immune cells comprising an engineered nucleic acid and/or engineered protein disclosed herein is administered to a subject in a number sufficient to decrease the size (e.g., diameter or mass), growth rate, rate of metastasis, and/or rate of recurrence of a tumor in the subject compared to a control. In some embodiments, the control is the size (e.g., diameter or mass), growth rate, rate of metastasis, and/or rate of recurrence of the tumor without administering the immune cell comprising the engineered nucleic acid and/or engineered protein disclosed herein. In some embodiments, the rate of recurrence is determined as the likelihood of recurrence of a given type of tumor in the absence of treatment with the immune cell comprising an engineered nucleic acid and/or engineered protein disclosed herein.

In some embodiments, any of the immune cells comprising an engineered nucleic acid and/or engineered protein disclosed herein is administered to a subject in a number sufficient to decrease the size (e.g., diameter or mass), growth rate, rate of metastasis, and/or rate of recurrence of a tumor in the subject compared to a control by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the size (e.g., diameter or mass), growth rate, rate of metastasis, and/or rate of recurrence of the tumor without administering the immune cell comprising the engineered nucleic acid and/or engineered protein disclosed herein.

In some embodiments, a method disclosed herein further comprises administering an immune checkpoint inhibitor to a subject. In some embodiments, the immune checkpoint inhibitor is a small molecule, peptide, protein (e.g., antibody, such as monoclonal antibody), interfering nucleic acid, or a combination of any of the foregoing. In some embodiments, the immune checkpoint inhibitor is a PD-1 inhibitor, PD-L1 inhibitor, a CTLA-4 inhibitor, or PD-L2 inhibitor. In some embodiments, an immune checkpoint inhibitor is a monoclonal antibody. See e.g., Wurz et al., Ther Adv Med Oncol. 2016 January; 8(1): 4-31; and Ann Oncol. 2015 December; 26(12):2375-91.

In some embodiments, the immune checkpoint inhibitor disrupts the interaction between PD-1 and PD-L1. In some embodiments, an immune checkpoint inhibitor is a PD-1 inhibitor. In some embodiments, the PD-1 inhibitor is an anti-PD-1 antibody. In some embodiments, the PD-1 inhibitor is pembrolizumab, nivolumab, pidilizumab, or cemiplimab. In some embodiments, the immune checkpoint inhibitor is a PD-L1 inhibitor. In some embodiments, the PD-L1 inhibitor is an anti-PD-L1 antibody. In some embodiments, a PD-L1 inhibitor is atezolizumab, avelumab, or durvalumab.

In some embodiments, an immune checkpoint inhibitor inhibits CTLA-4 (e.g., ipilimumab, and tremelimumab), IDO-1 (e.g., elotuzumab, INCB024360 and indoximod), KIR (such as lirilumab) or LAG-3 (e.g., IMP321 and BMS-986016).

The immune checkpoint inhibitor may be administered at the same time as an immune cell disclosed herein to the subject or the immune checkpoint inhibitor may be administered at a different time from an immune cell disclosed herein. The immune checkpoint inhibitor may be formulated with an immune cell disclosed herein or formulated separately.

In some embodiments, immune cells comprising an engineered nucleic acid and/or engineered protein disclosed herein and an immune checkpoint inhibitor are administered to a subject in a number sufficient to increase the number of tumor-infiltrating immune cells in a tumor of the subject compared to a control. In some embodiments, the control is the number of tumor-infiltrating immune cells in the tumor without administering the immune cell comprising the engineered nucleic acid and/or engineered protein disclosed herein and the immune checkpoint inhibitor.

In some embodiments, any of the immune cells comprising an engineered nucleic acid and/or engineered protein disclosed herein and an immune checkpoint inhibitor are administered to a subject in a number sufficient to decrease the size (e.g., diameter or mass), growth rate, rate of metastasis, and/or rate of recurrence of a tumor in the subject compared to a control. In some embodiments, the control is the size (e.g., diameter or mass), growth rate, rate of metastasis, and/or rate of recurrence of the tumor without administering the immune cell comprising the engineered nucleic acid and/or engineered protein disclosed herein and the immune checkpoint inhibitor. In some embodiments, the rate of recurrence is determined as the likelihood of recurrence of a given type of tumor in the absence of treatment with the immune cell comprising an engineered nucleic acid and/or engineered protein disclosed herein and the immune checkpoint inhibitor.

In some embodiments, any of the immune cells comprising an engineered nucleic acid and/or engineered protein disclosed herein and an immune checkpoint inhibitor is administered to a subject in a number sufficient to decrease the size (e.g., diameter or mass), growth rate, rate of metastasis, and/or rate of recurrence of a tumor in the subject compared to a control by at least 25% to at least 1000% (e.g., at least 25%, at least 50%, at least 75%, at least 100%, at least 200%, at least 300%, at least 400%, at least 500%, at least 600%, at least 700%, at least 800%, at least 900%, or at least 1000%, including all values in between) compared to a control. In some embodiments, the control is the size (e.g., diameter or mass), growth rate, rate of metastasis, and/or rate of recurrence of the tumor without administering the immune cell comprising the engineered nucleic acid and/or engineered protein disclosed herein and the immune checkpoint inhibitor.

### Kits

Also encompassed by the disclosure are kits (e.g., pharmaceutical packs). The kits provided may comprise an engineered nucleic acid, engineered protein, composition, and/or immune cell comprising an engineered nucleic acid and/or engineered protein disclosed herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition described herein. In some embodiments, the pharmaceutical composition described herein provided in the first container and the second container are combined to form one unit dosage form.

Thus, in one aspect, provided are kits including a first container comprising a pharmaceutical composition described herein. In certain embodiments, the kits are useful for treating a cancer in a subject in need thereof. In certain embodiments, the kits are useful for preventing a cancer in a subject in need thereof. In certain embodiments, the kits are useful for reducing the risk of developing a cancer in a subject in need thereof. In certain embodiments, the kits are useful for increasing the anti-tumor activity of an immune cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a cancer in a subject in need thereof. In certain embodiments, the kits and instructions provide for preventing a cancer in a subject in need thereof. In certain embodiments, the kits and instructions provide for reducing the risk of developing a cancer in a subject in need thereof. In certain embodiments, the kits and instructions provide for increasing the anti-tumor activity of an immune cell. A kit described herein may include one or more additional pharmaceutical agents described herein as a separate composition.

### EXAMPLES

### Example 1: PPARδ Activation in Immune Cells Promoted Anti-Tumor Immunity

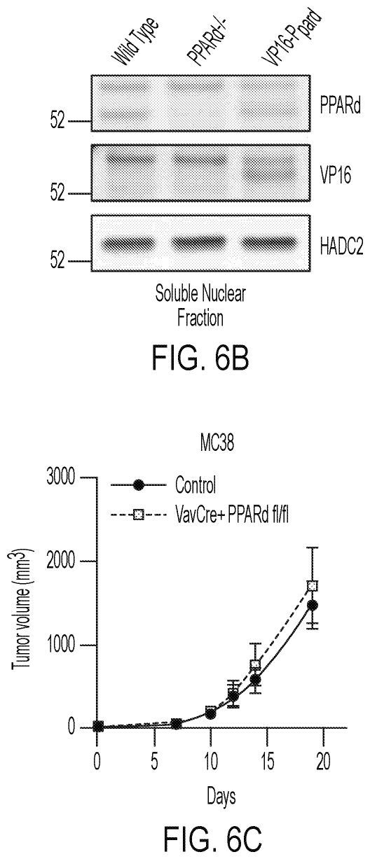

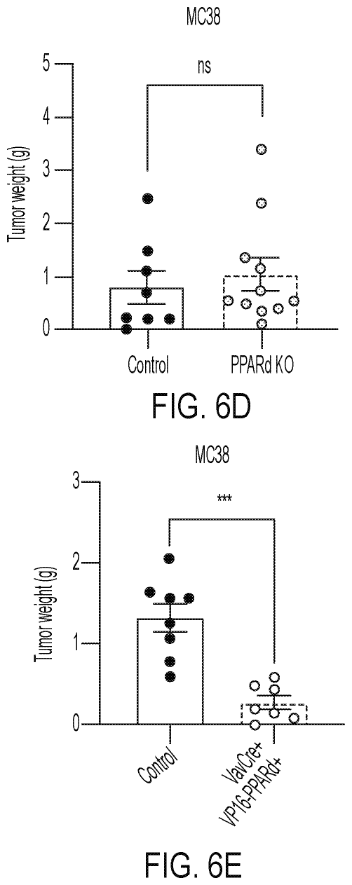

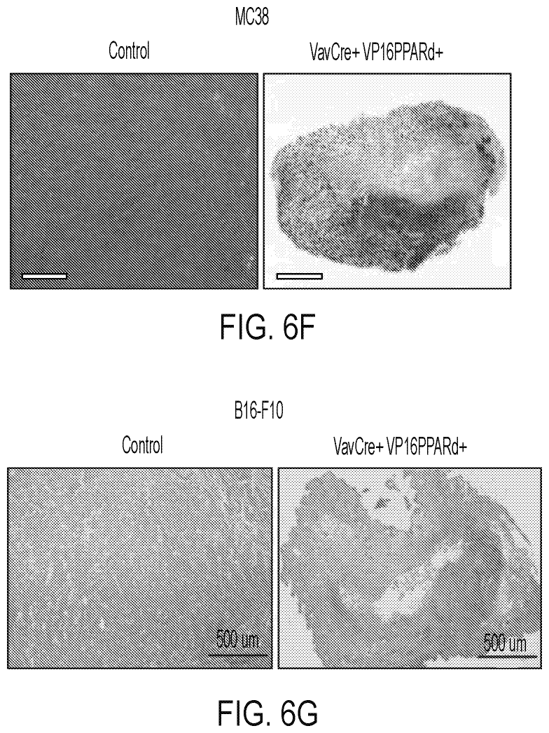

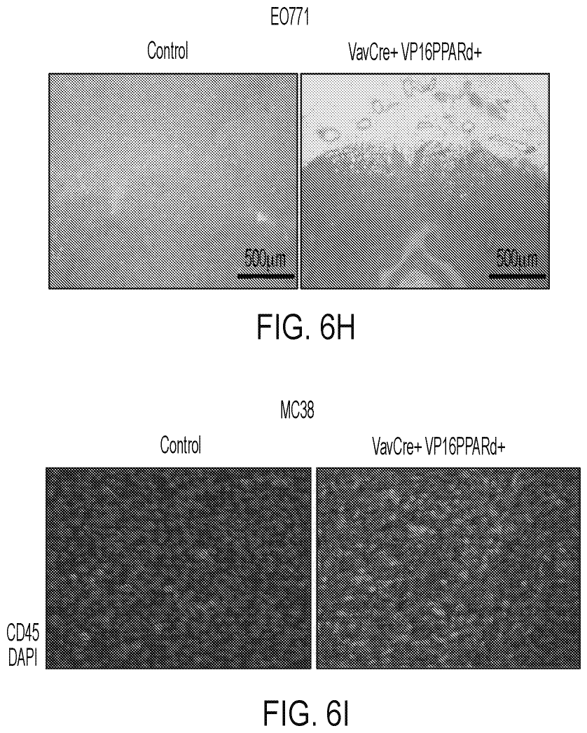

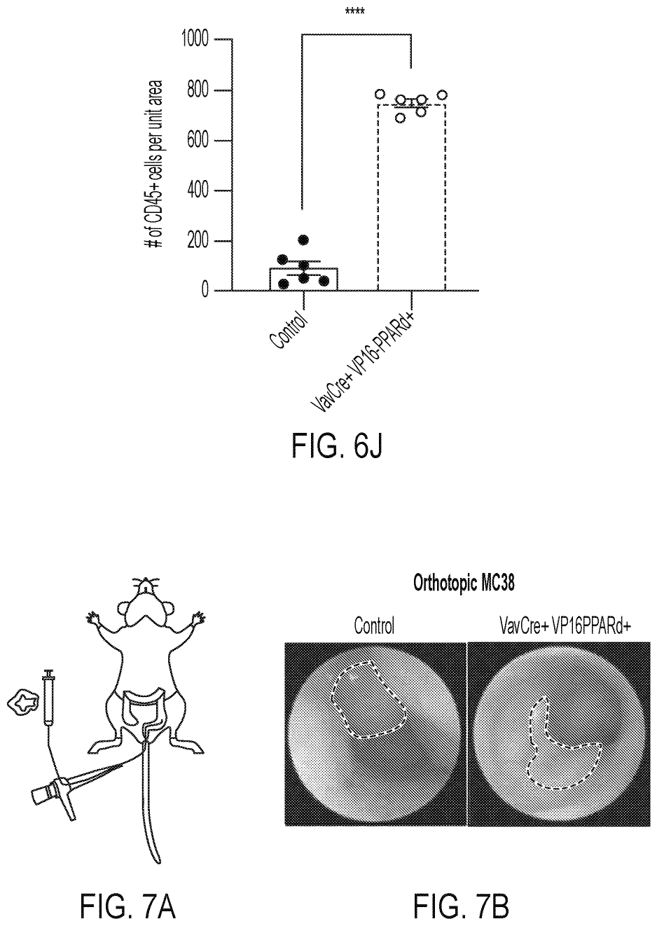

To determine the necessity and sufficiency of PPARδ signaling for immune cell function in vivo, a novel loss-of-function (LOF) and gain-of-function (GOF) mouse models driven by vav-cre were generated. Vav-cre targets all hematopoietic cells and was used to analyze the overall effects of PPARδ LOF or GOF on all immune cells. To test the necessity of PPARδ for immune cell function, an immune cell specific PPARδ knock-out (KO) mouse model “vav-cre+ PPARδ^{fl/fl}” was generated. Successful PPARδ knock-out at mRNA and protein levels using real-time quantitative reverse transcription polymerase chain reaction (qPCR) and western blot, respectively, were confirmed (FIGS. 6A and 6B). In order to assess the necessity of PPARδ for anti-tumor immunity, vav-cre+ PPARδ^{fl/fl} mice were challenged with subcutaneous MC38 colorectal adenocarcinoma tumors (see, e.g., Corbett et al., 1975). Tumor growth was followed for 20 days and comparable MC38 tumor sizes between wild-type and vav-PPARδ KO mice were found (FIGS. 6C and 6D). This suggests PPARδ is dispensable for anti-tumor immune response. Without being bound by a particular theory, PPARδ may be dispensable for anti-tumor immune response due to compensation by another PPAR family member PPARα, which promotes similar fatty acid catabolism pathways to PPARδ.

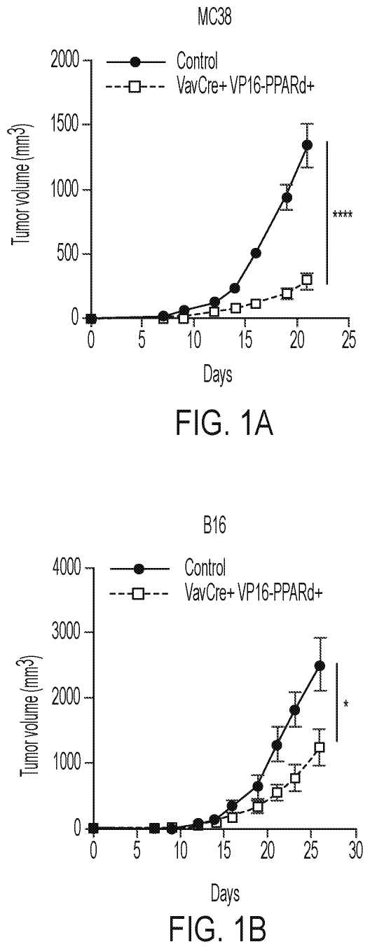

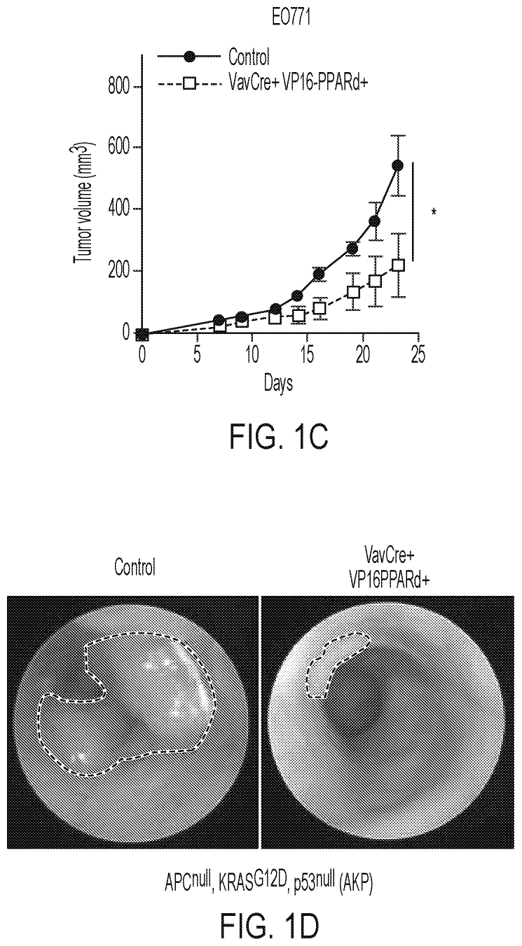

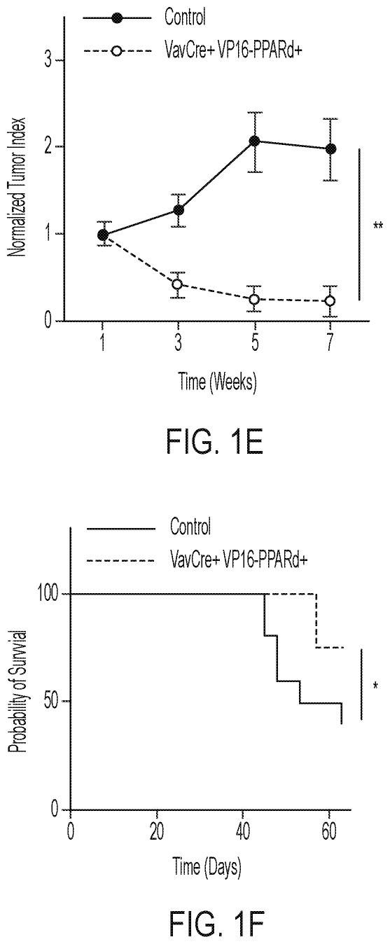

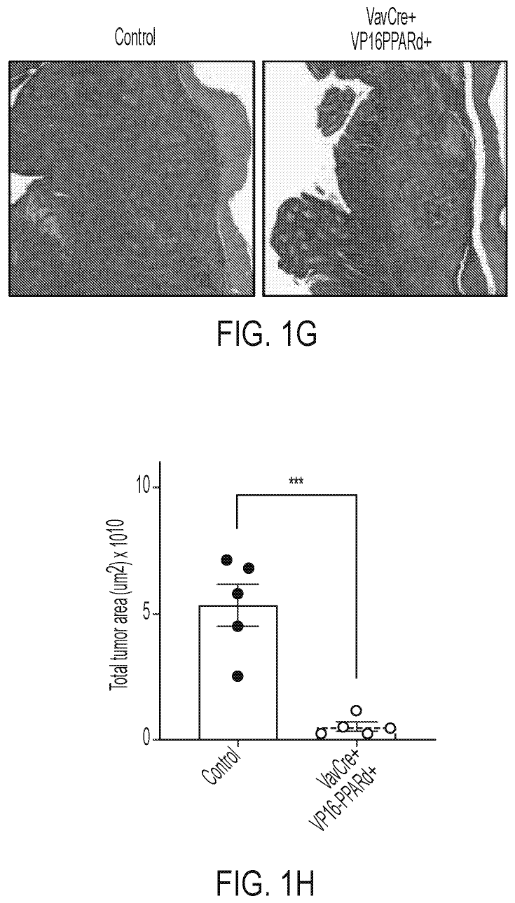

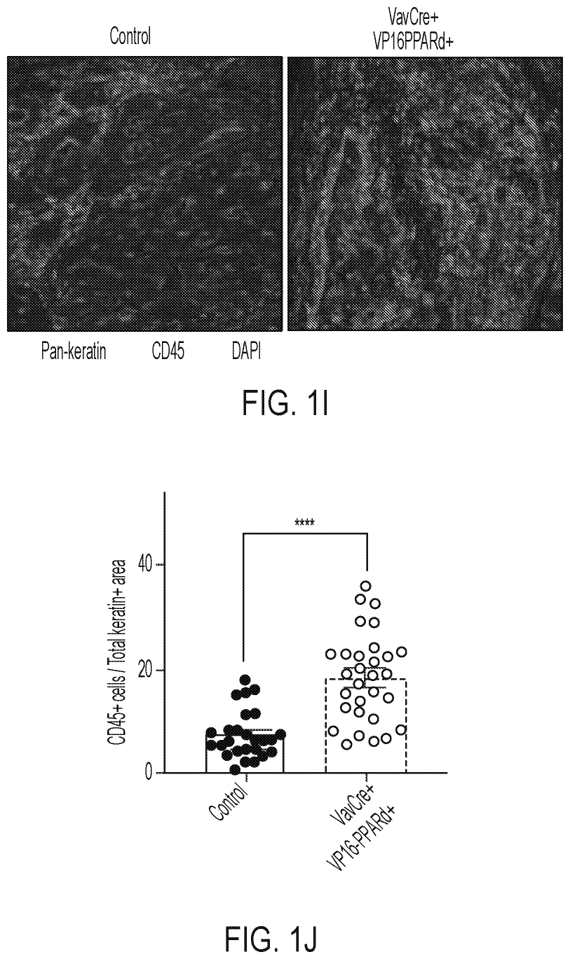

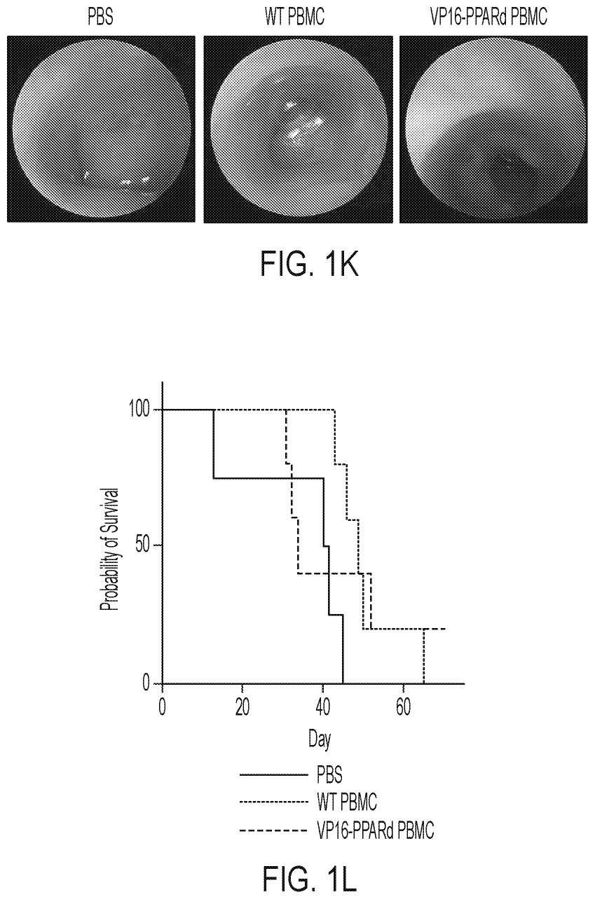

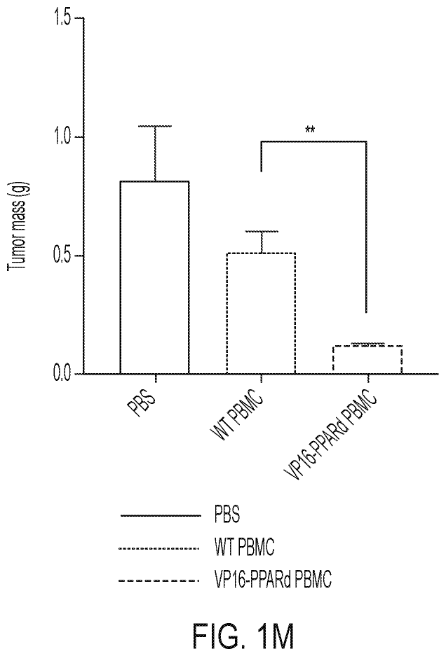

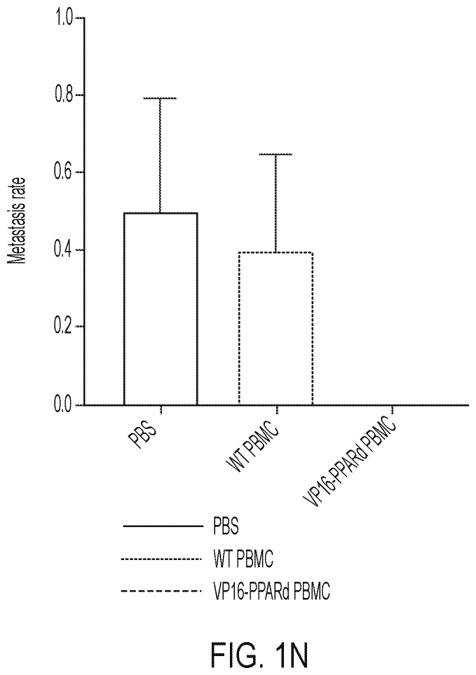

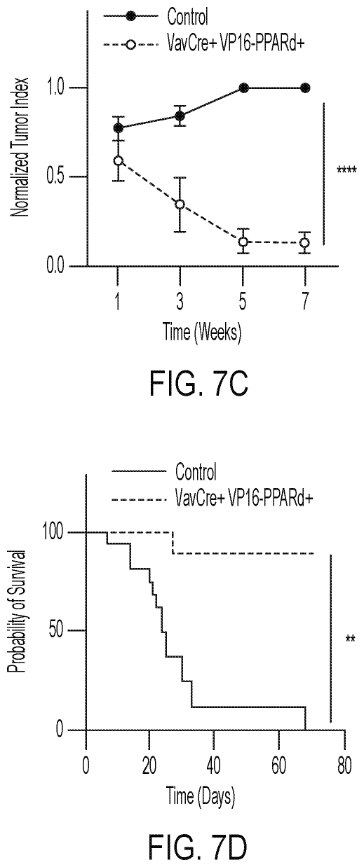

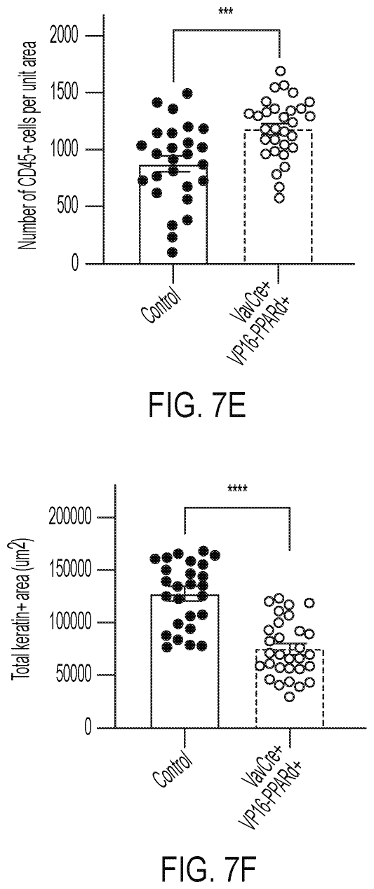

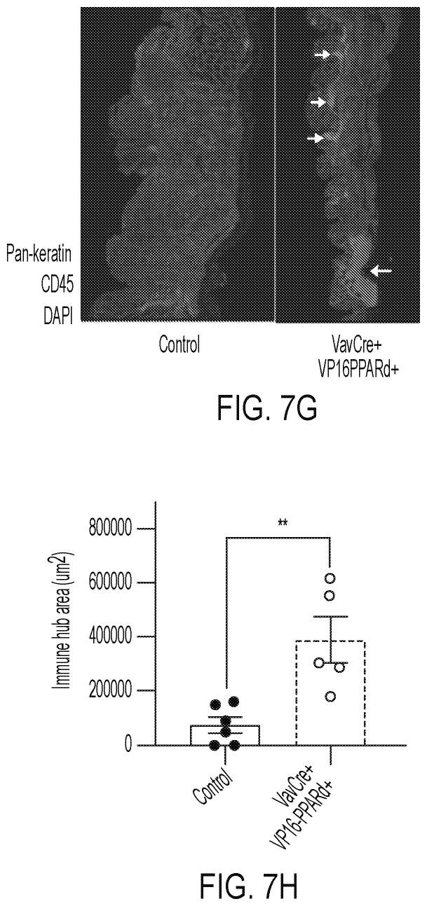

In order to determine the effects of PPARδ gain-of-function on immune cell function, mouse model was generated that overexpressed a constitutively active PPARδ fusion protein VP16-PPARδ in all immune cells, driven by vav-cre (“vav-cre+ VP16-PPARδ+”). Constitutive PPARδ activation was achieved by fusing wild-type PPARδ to VP16 transactivation domain from herpes simplex virus. The presence of VP16 domain in splenocytes isolated from vav-cre+ VP16-PPARδ+ mice was confirmed using western blot (FIG. 6B). To assess anti-tumor immune response upon PPARδ activation in immune cells, MC38 colorectal adenocarcinoma cell line was subcutaneously injected, which gives rise to highly immunogenic tumors (see, e.g., Corbett et al., 1975; Efremova et al., 2018). Intriguingly, established MC38 tumors in vav-cre+ VP16-PPARδ+ mice were either rejected around days 12-14 after tumor injection, or grew significantly smaller compared to tumors in wild-type mice (FIGS. 1A, 6E, and 6F). To corroborate these observations, endoscopy-guided orthotopic transplantation models of colorectal cancer were used (see, e.g., Beyaz et al., 2016; Beyaz et al., 2020; Roper et al., 2017). Similar to the subcutaneous model, PPARδ activation in immune cells led to a significant reduction in tumor burden in orthotopic MC38 tumors and drastically improved survival compared to control mice (FIGS. 7A-7D).

To determine if PPARδ gain-of-function in immune cells would be sufficient to control the growth of less immunogenic cancer cell lines, aggressive and poorly immunogenic cancer cell lines B16-F10 and EO771 were used (see, e.g., Ewens et al., 2005; Fidler, 1975; Pan et al., 1999; Sugiura and Stock, 1952). B16-F10 melanoma cells were subcutaneously injected into the right flanks and orthotopically injected EO771 breast carcinoma cells into mammary fat pads of vav-cre+ VP16-PPARδ+ mice. B16-F10 and EO771 tumors grew significantly smaller in vav-cre+ VP16-PPARδ+ mice, despite their poor immunogenicity (FIGS. 1B, 1C, 6G, and 6H). These results suggest that genetic PPARδ gain-of-function in immune cells impairs the growth of subcutaneous and orthotopic tumors derived from murine cancer cell lines, irrespective of tumor type, immunogenicity or location.

Cancer immunotherapies such as immune checkpoint blockade (ICB) have shown unparalleled response rates in several cancer types including melanoma, non-small-cell lung cancer and renal cell carcinoma (Sharma et al., 2017). ICB has also significantly improved the treatment of “microsatellite instable” (MSI) colorectal cancer (CRC), a small subset of colorectal cancer that exhibits defects in DNA mismatch repair (MMR) and thus enriched with neoantigens (Sahin et al., 2022). However, around 50% of MSI colorectal cancers do not respond to immunotherapy, for reasons that remain unclear (Le et al., 2017). How tumor mutational burden (TMB) and thus neoantigen burden impact cancer immunotherapy response remains elusive (Gurjao et al., 2020; Westcott et al., 2021). In addition, 85% of CRC tumors are “microsatellite stable” (MSS). MSS tumors are poorly infiltrated with effector immune cells (“immune cold”) and do not respond to ICB (Picard et al., 2020). MSS tumors were shown to be enriched for mutations in tumor suppressor gene APC, a key regulator of Wnt signaling, and proto-oncogene KRAS, which regulates cellular processes such as proliferation and survival (Grasso et al., 2018). Activation of Wnt signaling through loss of APC and oncogenic KRAS mutations have been correlated with immune exclusion (Grasso et al., 2018; Hamarsheh et al., 2020; Luke et al., 2019). In order to assess the effects of PPARδ activation in a physiologically relevant colorectal cancer model, APC^{null}, KRAS^{G12D}, P53^{null} (AKP) organoids, a genetically engineered murine model (GEMM) of MSS colorectal carcinoma (see, e.g., Roper et al., 2018) was used. Endoscopy guided orthotopic injections were used to inject AKP organoids into colon submucosa and closely followed tumor growth using colonoscopy imaging (see, e.g., Roper et al., 2018). Strikingly, PPARδ activation in immune cells was sufficient to eradicate orthotopically established organoid-derived AKP carcinomas and improved survival compared to control mice (FIGS. 1D-1H).

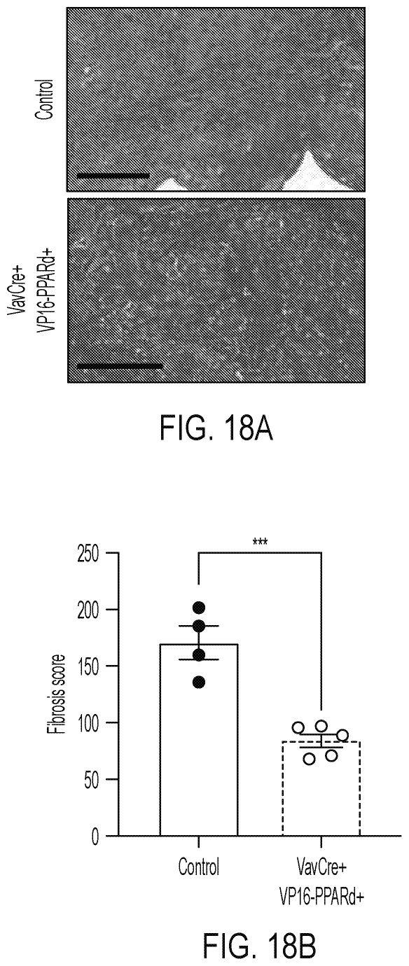

In order to dissect the anti-tumor immune response in vav-cre+ VP16-PPARδ+ mice, the presence and spatial distribution of tumor infiltrating immune cells in subcutaneous and orthotopic models of colorectal cancer was analyzed. It was determined that PPARδ gain-of-function in immune cells significantly increased the number of tumor-infiltrating immune cells in subcutaneous MC38 tumors (FIGS. 61 and 6J). This could indicate increased immune cell migration to tumor site, as well as enhanced survival or proliferation of tumor infiltrating immune cells. Furthermore, orthotopic AKP tumors grown in wild-type mice exhibited glandular formations that are characteristic of colorectal adenocarcinomas. Immune cells in AKP tumor beds were excluded from these tumor glands in control mice, recapitulating immune cold phenotype of MSS CRCs (FIG. 1I). Remarkably, PPARδ activation in immune cells successfully eliminated the glandular structures and significantly increased the number of tumor infiltrating immune cells in AKP tumors (FIGS. 1I, 1J, 7E, and 7F). Furthermore, PPARδ activation in the immune system drastically decreased the presence of fibrotic areas in AKP tumors, indicating faster resolution of tumor-related inflammation (FIGS. 18A-18B). In addition, large clusters of immune cells (“immune hubs”) adjacent to AKP tumors in vav-cre+ VP16-PPARδ+ mice were identified (FIGS. 7G and 7H). Immune hubs are spatially organized networks of immune cells within or near tumors that are argued to promote anti-tumor responses (Pelka et al., 2021). Taken together, these results suggest that PPARδ activation in immune cells is a single immune cell intrinsic switch that is sufficient to override immune exclusion and promote anti-tumor immunity irrespective of tumor immunogenicity.

FIGS. 1K-1N show that PPARδ activation promotes anti-tumor immunity and reduces metastasis rate in immunosuppressive metastatic colorectal carcinoma. Metastatic colorectal adenocarcinoma organoids APC^{null} Kras^{G12D} p53^{null} Smad4^{null} (AKPS) (See, e.g., Westcott et al., Nat. Cancer, 2021) were orthotopically injected to WT mice that received PBS, WT PBMCs or VP16-PPARδ+ PBMCs prior to tumor inoculation. FIG. 1K shows representative colonoscopy images 3 weeks after tumor inoculation. FIG. 1L shows survival curve of AKPS tumor bearing mice that received PBS, wild-type PBMCs, or VP16-PPARδ PBMCs prior to tumor inoculation. FIG. 1M shows AKPS tumor weights at humane end points (40-60 days post tumor inoculation). FIG. 1N shows the rate of lung, liver or omentum metastases in AKPS tumor bearing mice that received PBS, wild-type PBMCs, or VP16-PPARδ PBMCs prior to tumor inoculation. The rate of metastasis was calculated per mouse. If metastasis was present in a given mouse, a score of 1 was given. If metastasis was not present in a given mouse, a score of 0 was given. The metastasis score for each mouse was then averaged. An average score of 1 corresponds to a metastasis rate of 100%. An average score of 0 corresponds to a metastasis rate of 0.

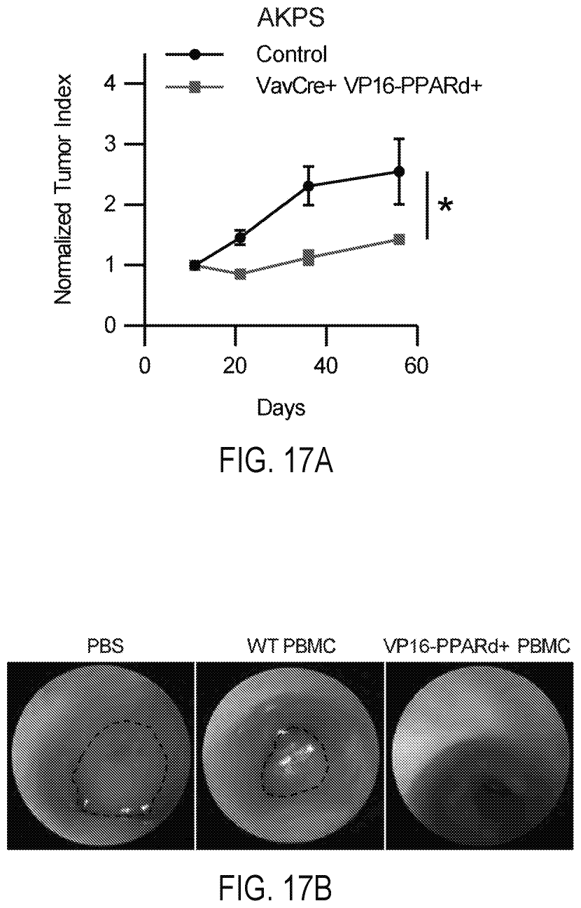

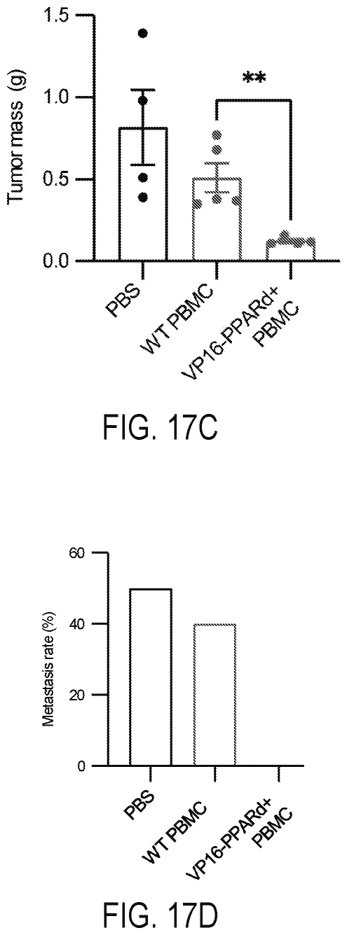

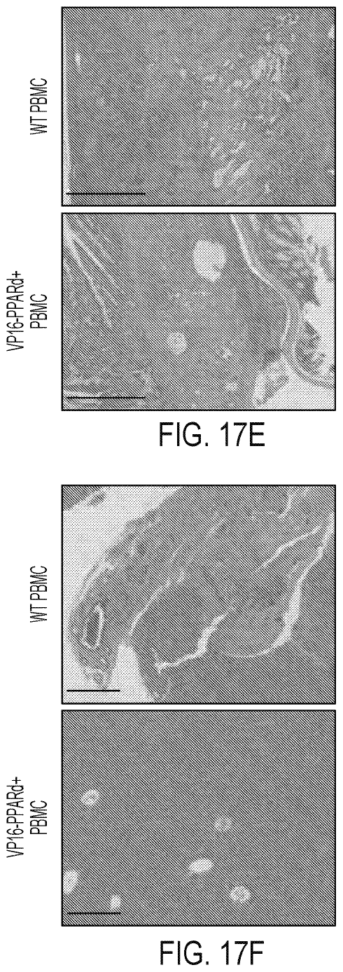

Mice with immune intrinsic expression of active PPARδ displayed significantly reduced growth of orthotopically transplanted AKPS tumors (FIG. 17A). Moreover, it was found that adoptive transfer of VP16-PPARδ+ peripheral blood mononuclear cells (PBMCs) to wild-type mice prior to orthotopic transplantation of MSS metastatic AKPS organoids dampens primary tumor growth and restricts metastasis (FIGS. 17B-17F).

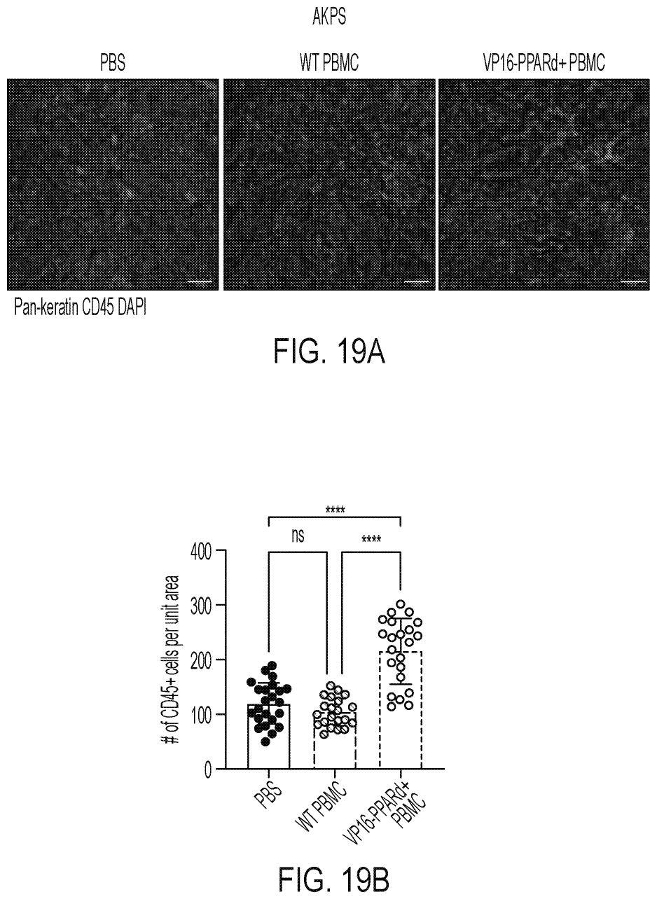

Moreover, while orthotopic AKPS tumors grown in wild-type mice displayed immune-excluded glandular structures, PPARδ activation in immune cells was sufficient to eliminate tumor glands and significantly increased the number of TILs in AKPS tumors (FIGS. 19A-19B).

Collectively, these findings suggests that immune-intrinsic PPARδ activation prevents tumor growth across different cancer types and stages, independent of antigenicity status.

Next, the possible contributions of immune cell extrinsic factors such as tumor associated fibroblasts to PPARδ mediated anti-tumor immunity were assessed. Accordingly, the direct immune cell intrinsic effects of PPARδ gain-of-function using an in vitro 2D co-culture system was studied. PBMCs were isolated from wild-type and vav-cre+ VP16-PPARδ+ mice, and treated wild-type PBMCs with PPARδ agonist GW501516 for 72 hours. Next, PBMCs were co-cultured with MC38 cells that express luciferase. Luciferase enzyme catalyzes the oxidation of its substrate luciferin, producing bioluminescence that is proportional to the amount of luciferase enzyme (Thorne et al., 2010). After 72 hours of co-culture, at effector-to-target ratio 5:1, GW501516 treated PBMCs and VP16-PPARδ+ PBMCs exhibited significantly higher in vitro killing activity, measured by adding luciferin and using loss of luminescence as a proxy for luciferase expressing cancer cell lysis (FIG. 7I). Thus, cell-intrinsic PPARδ activation in murine PBMCs was sufficient to promote anti-tumor effector function in vitro.

### Additional Materials and Methods

The following materials and methods were used to generate the data shown in the indicated figures.
FIG. 17A—Orthotopic AKPS Implantation into VavCre+ VP16-PPARd+ Mice:

### Mouse Intestinal Organoid Culture

AKPS [APC KO, Kras^{G12D}, p53 KO, Smad4 KO (Westcott et al., Nature Cancer 2021 2:10. 2021 Sep. 30; 2(10):1071-85)] organoids were embedded in Matrigel (Corning, 356234) and cultured with minimal organoid media [Advanced DMEM F-12 (Gibco, 12634028) supplemented with N2 (Thermo Fisher, 17502048) and B27 (Thermo Fisher 17504044)], as described previously (Roper et al., Nature Biotechnology 2017 35:6. 2017 May 1; 35(6):569-76). Organoids were split using TryplE Express (Thermo Fisher, 12604) every three days.

### Colonoscopy Guided Injections

Orthotopic injections of colorectal cancer organoids and cell lines were performed as described previously (Roper et al., Nat Protoc. 2018 Feb. 1; 13(2):217-34; Beyaz et al., Nature. 2016 Mar. 2; 531(7592):53-8). Briefly, organoids were collected by gentle scraping and separated from Matrigel in Cell Recovery Solution (Corning, 354253), rotating for 40 minutes at 4° C. In order to estimate the number of cells in organoids, a fraction of organoids was dissociated in TrypLE Express enzyme and incubated at 37° C. for 30 minutes, followed by cell counting. Cell concentration was adjusted to 10×10⁶, and intact organoids were resuspended in minimal organoid media with 10% Matrigel. Organoids were injected into colon sub-mucosa using a Hamilton syringe (7656-1) and a custom 33G needle (Hamilton, custom made similar to 7803-05, 16”, Pt 4, Deg 12). Each mouse received 100 ul organoids (that contain approximately 1×10⁶ cells of AKPS organoids). Successful injections were confirmed by observing large bubbles in the colon mucosa. Tumor growth was monitored using colonoscopy. Tumor index was calculated by dividing the tumor diameter by the colon diameter using colonoscopy images. Each tumor index was normalized to their respective week-1 tumor index.
   FIGS. 17B-17F—VP16-PPARd+ PBMC Transfer with AKPS Transplantation
C57BL/6J mice were sub-lethally irradiated using 5 Gy total body irradiation. Splenocytes were isolated from control or vav-cre+ VP16-PPARδ+ mice and cultured in complete RPMI (RPMI (Corning, 10-040-CV) supplemented with 10% fetal bovine serum (FBS), 1% penicillin-streptomycin (P/S), 50 uM β-mercaptoethanol (MilliporeSigma, M3148-25ML)). 48 hours later, mice received intravenous injections of 5×10⁶ splenocytes. 72 hours later, 2×10⁶ AKPS cells were injected into colon sub-mucosa as described above. Mice underwent colonoscopy 3 weeks later to assess tumor formation and generate representative images. Mice were euthanized at humane endpoint. Tumor weight was measured at endpoints and metastasis to liver, lung and omentum was assessed.

### FIGS. 18A-18B—Masson's Trichome Staining

Fibrotic areas were determined by an expert pathologist in a blinded manner using FFPE tumor slides stained with Masson's trichrome stain. Aperio ImageScope color deconvolution algorithm was used to determine fibrosis on the blue color channel. The total percent of fibrosis was assigned a score based on medium and strong blue pixels. The maximum score was 300.
   FIGS. 19A-19B—Immunofluorescence from PBMC Transfer Experiment
C57BL/6J mice from the experiment in FIGS. 17B-17D were euthanized when reaching humane endpoint. Tumor tissues were fixed in 10% formalin for 24 hours, washed in 70% ethanol and embedded in paraffin. 10 um sections were mounted on slides. For immunofluorescent staining, paraffin was removed using xylene and tissues were gradually re-hydrated. Antigen retrieval was performed in Tris-EDTA Buffer (10 mM Tris, 1 mM EDTA, 0.05% Tween-20, pH=9) in a pressure cooker at 110° C. for 10 minutes. Tissues were blocked with SuperBlock blocking buffer (Thermo Fisher, 37515) for 1 hour at room temperature (RT), and sequentially stained with primary antibodies [CD45 (CST, clone D3F8Q, dilution 1:500), Pan-keratin (CST, clone 4545T, dilution 1:250)] overnight at 4° C. and secondary antibodies (Thermo Fisher, A32790, A21208, A31573 or A48272, dilution 1:500) for 1 hour at RT. Tissues were stained with 0.5 ug/ml DAPI (Invitrogen, D3571) for 10 minutes, mounted with ProLong™ Diamond Antifade Mountant (Thermo Fisher, P36970) and imaged with Zeiss LSM710 confocal microscope, using a Plan-Apochromat 10×/0.45 M27 Air lens and Plan-Apochromat 20×/0.8 M27 Air lens. Images were processed with FIJI (ImageJ2, Version 2.3.0) (Rueden et al., BMC Bioinformatics. 2017 Nov. 29; 18(1):1-26). After Z-projection and brightness/contrast adjustment in FIJI, image quantification was performed in QuPath (Bankhead et al., Scientific Reports. 2017 Dec. 4; 7(1):1-7). For quantification of cells (CD45+ cells) QuPath's automatic positive cell detection, manual counting or a combination of these methods was used.

### Example 2: PPARδ Activation in Immune Cells Remodels the Tumor Microenvironment into a Pro-Inflammatory State

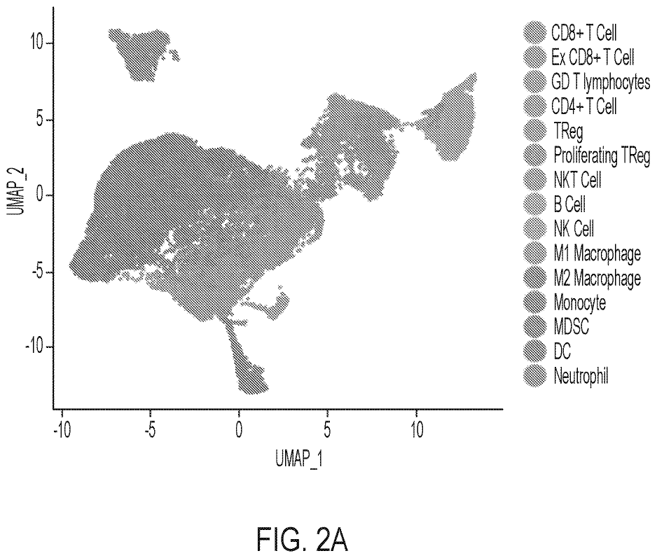

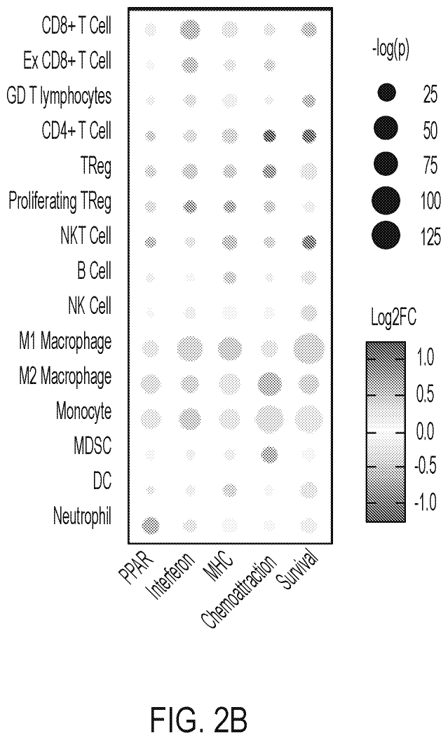

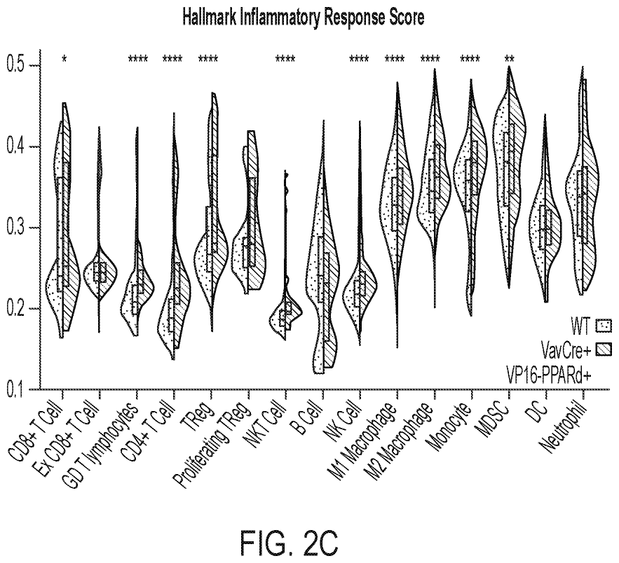

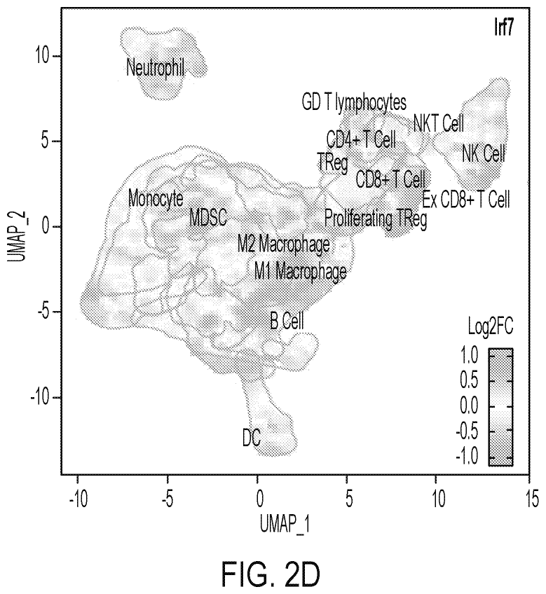

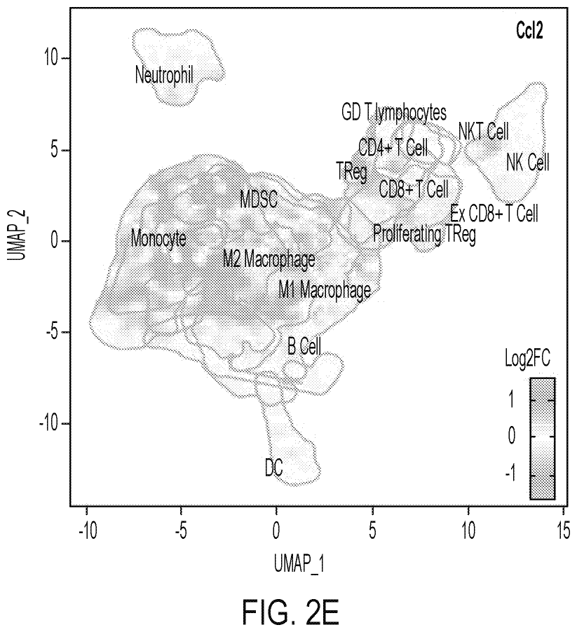

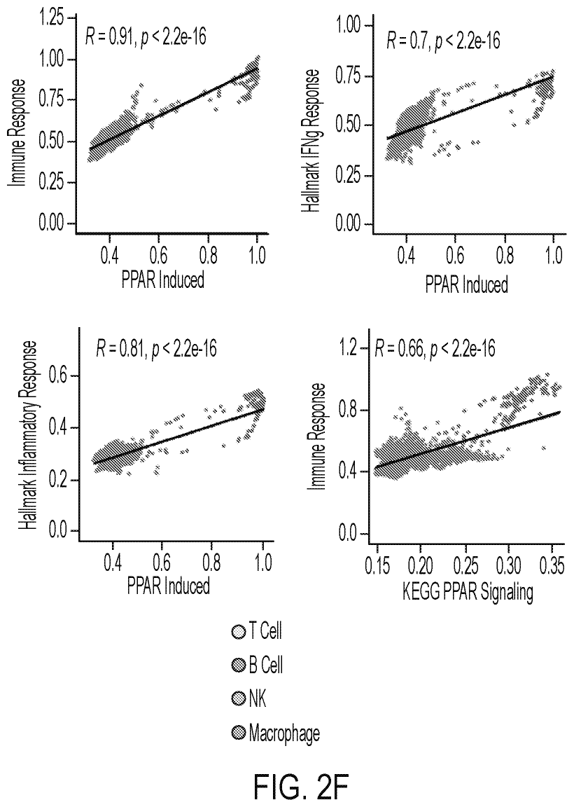

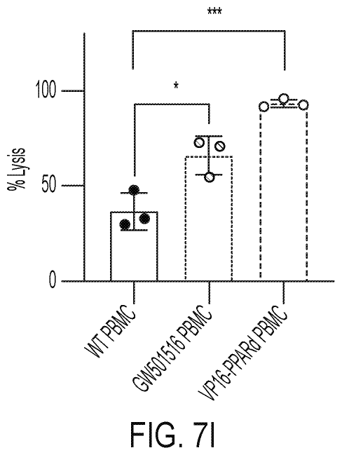

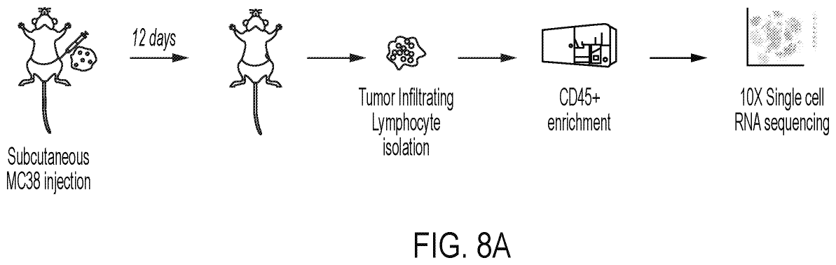

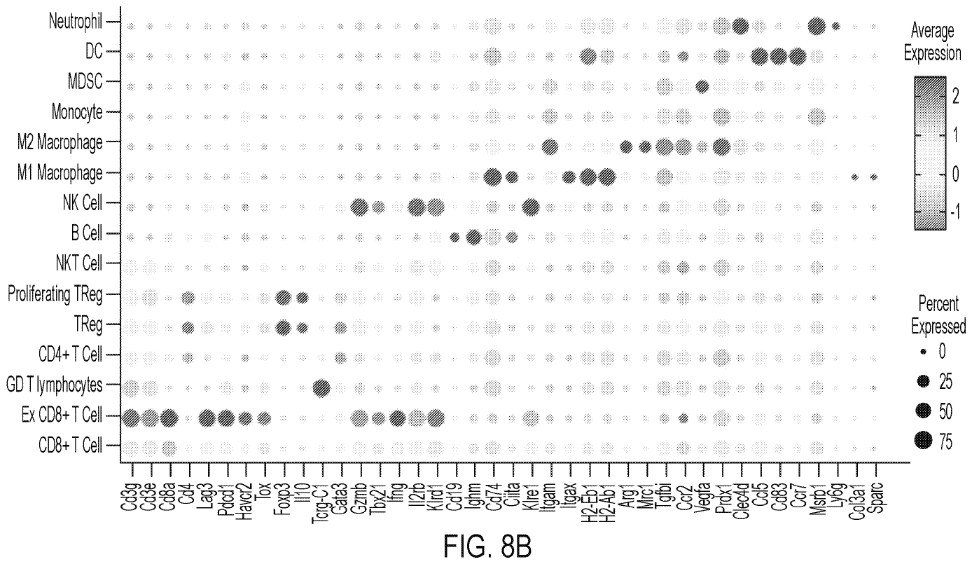

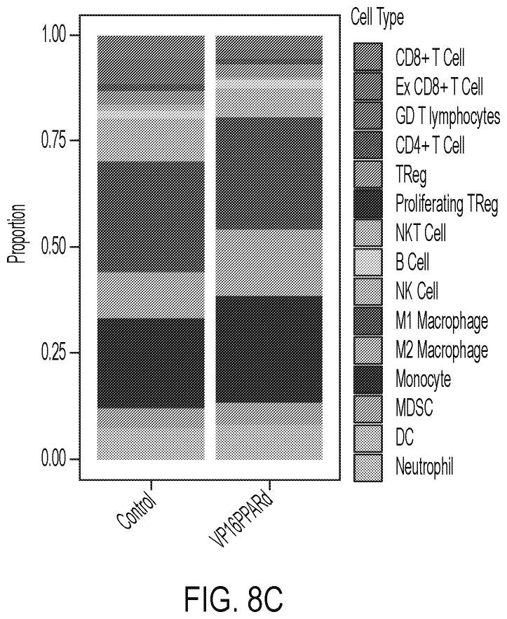

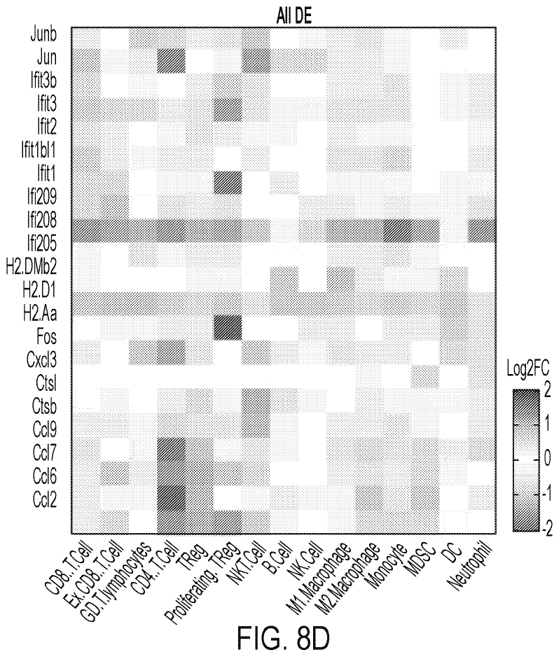

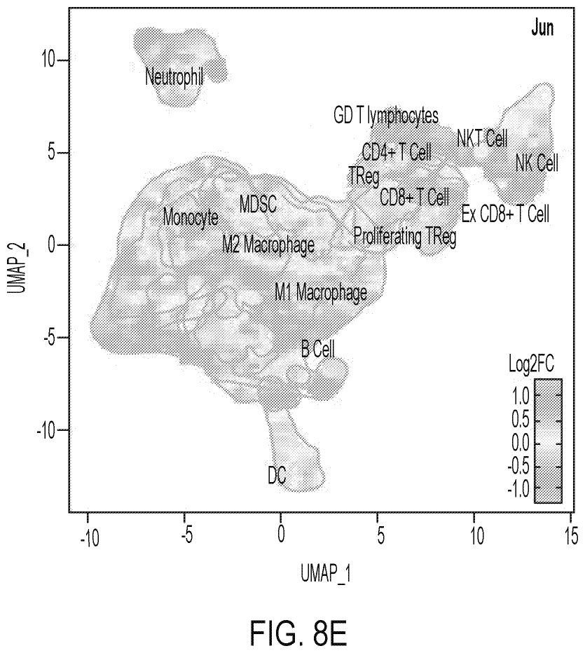

To assess the transcriptional state of tumor-infiltrating immune cells prior to tumor rejection, MC38 were subcutaneously injected cells into control and vav-cre+ VP16-PPARδ+ mice. 12 days after tumor injection, when tumor sizes were comparable, CD45+ tumor infiltrating immune cells (TILs) were isolated using fluorescent activated cell sorting (FACS) and performed single cell RNA sequencing (scRNA-seq). The clustering analysis identified major populations of lymphoid and myeloid tumor infiltrating immune cells (FIGS. 2A and 8A-8C).

Analysis of top differentially expressed genes across all TILs revealed significant upregulation of genes involved in interferon response (Ifi208, Irf7, Ifit1), antigen presentation (H2-D1, Ctsb), chemoattraction (Ccl2, Ccl9) and survival (Jun, Fos) (FIGS. 2B, 8D, and 8E). (see, e.g., Embgenbroich and Burgdorf, 2018; Honda et al., 2005; Kronke et al., 2007; Landolfo et al., 1998; Li et al., 2022; Pidugu et al., 2019; Shaulian and Karin, 2001). As these processes are key components of robust inflammatory responses, the expression of “hallmark inflammatory response” genes was scored across all TILs (see, e.g., Liberzon et al., 2015). Remarkably, PPARδ activation in immune cells increased expression of hallmark inflammatory response genes in not only pro-inflammatory cell types such as CD8 T cells and natural killer (NK) cells, but also in tolerogenic, immunosuppressive cell types such as M2-like macrophages, myeloid derived suppressive cells (MDSCs) and regulatory T cells (T_{reg}) (FIG. 2C).

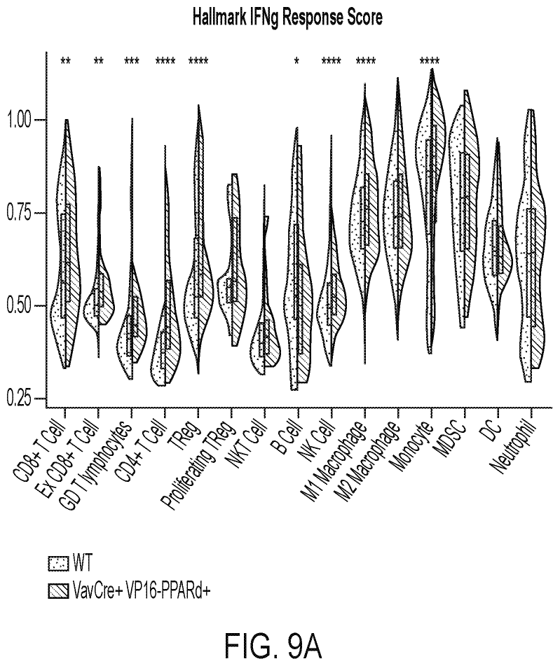

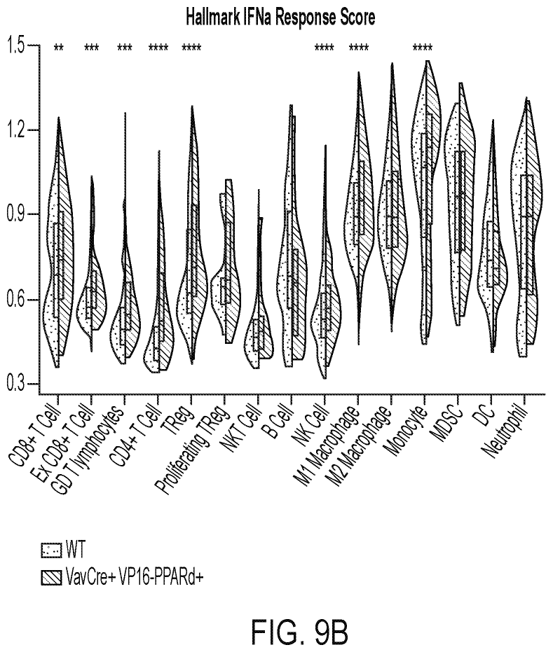

Interferons (IFN) are key pro-inflammatory cytokines that are involved in anti-viral and anti-tumor immunity. Interferons can exert their anti-tumor effects through direct tumor intrinsic mechanisms as well as regulating the function of tumor infiltrating immune cells. Type I interferons IFN-α and IFN-β are mainly produced by dendritic cells but can be secreted by almost all cell types. Type II interferon IFN-γ is mainly produced by natural killer (NK) cells and cytotoxic CD8 T cells. Interferon binding to interferon receptors (IFNAR) on cell surface activates Janus kinase—signal transducer and activator of transcription (JAK-STAT) signaling pathway and regulates the expression of various IFN-inducible genes (Parker et al., 2016). Interferon regulatory factor 7 (Irf7) is the master regulator of type I IFN responses (Honda et al., 2005). Irf7 and its downstream type I IFN pathways were previously demonstrated to promote anti-tumor immunity (Bidwell et al., 2012). Intriguingly, PPARδ gain-of-function in immune cells promoted Irf7 expression across TILs (FIG. 2D). In addition, IFN-α inducible chemokine Ccl2 plays a critical role on inflammatory immune cell recruitment, and was upregulated in myeloid clusters upon PPARδ gain-of-function (Conrady et al., 2013) (FIG. 2E). Furthermore, IFNs can promote the expression of major histocompatibility complexes (MHC), thereby regulating antigen presentation (see, e.g., Steimle et al., 1994; Zhou, 2009). Several genes in the murine H2 locus (H2.DMb2, H2.D1, H2.Aa) were upregulated upon PPARδ gain-of-function. H2 locus encodes for MHC Class I and II molecules, which can indicate enhanced antigen presentation upon PPARδ activation (FIG. 8D). Upon noting significant upregulation of various IFN inducible genes, the expression of hallmark IFN-α and IFN-γ response genes were scored (see, e.g., Liberzon et al., 2015). PPARδ activation in immune cells induced robust upregulation of genes involved in IFN-α and IFN-γ responses across TILs (FIGS. 9A and 9B).

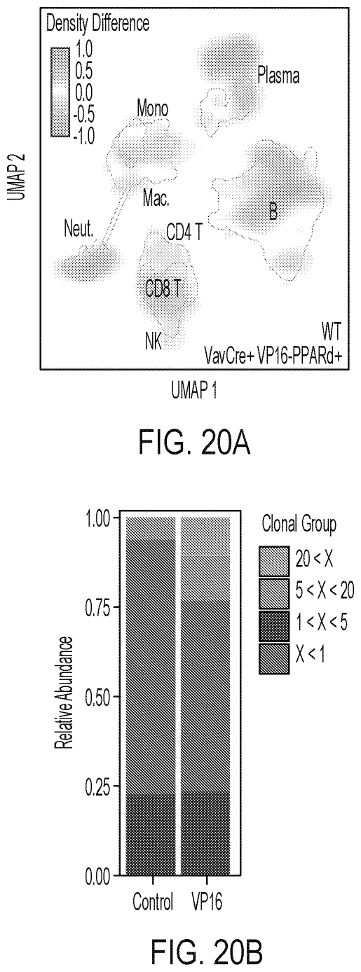

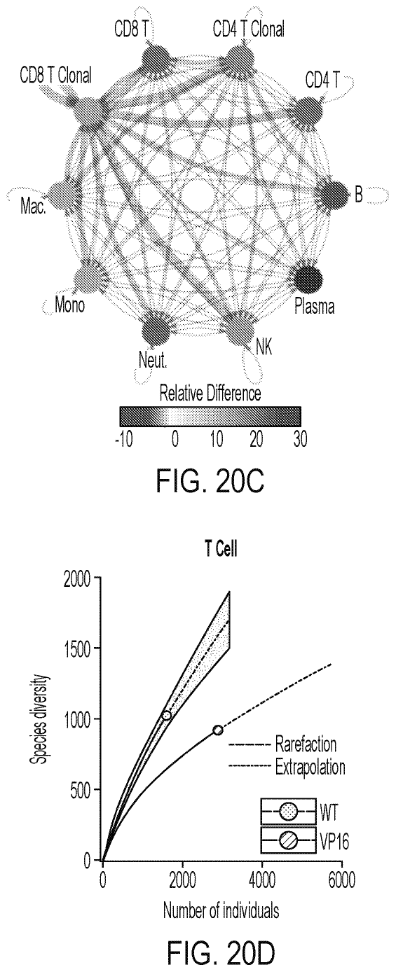

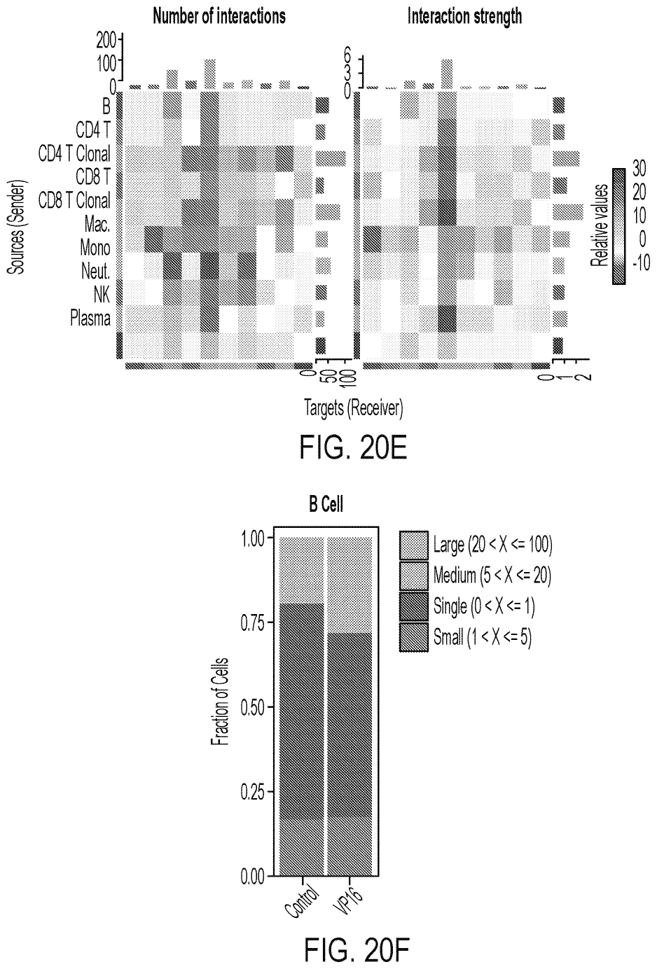

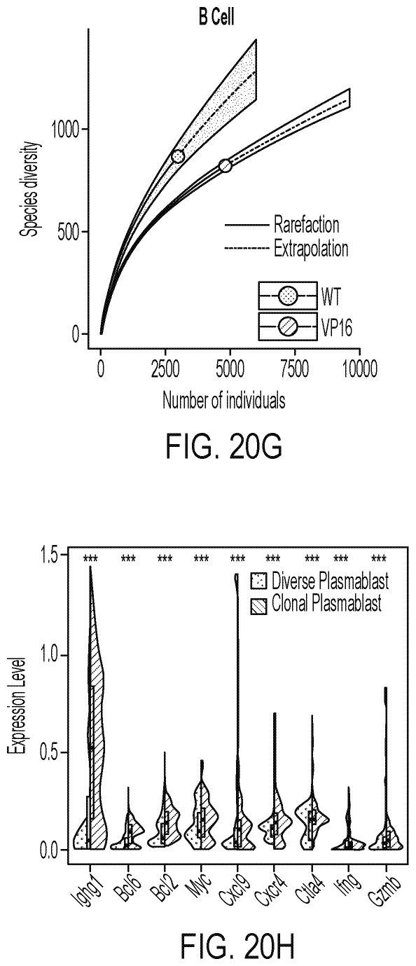

Notably, cell type abundance revealed that AKPS tumors in mice with immune-intrinsic PPARδ activation showed increased numbers of effector immune cell types including plasmablasts, and CD8 T-cells (FIG. 20A). Analyzing TCR sequences from tumor-infiltrating T-cells, it was found that clonal expansion of T-cells, indicative of proper T-cell activation and function, was largely absent in control mice, whereas vavCre+ VP16-PPARd+ mice displayed clonally expanded T-cell populations (FIGS. 20B and 20D). Notably, such clonally expanded CD8 and CD4 T-cells are predicted to interact more with other immune cell types within the tumor microenvironment (FIGS. 20C and 20E). Clonal expansion was also observed within the B-cell compartment (FIGS. 20F-20H). In sum, these analyses support the notion that PPARδ activation enhances survival and fitness of immune cells, leading of increased functional capacities within the TME. Taken together, these results suggest that PPARδ gain-of-function in all immune cells remodels the tumor microenvironment (TME) into a pro-inflammatory state. Anti-inflammatory features of the TME such as immunosuppressive immune cells, immunosuppressive cytokines, and anti-inflammatory metabolites are major barriers to anti-tumor immunity and cancer immunotherapy response (Sharma et al., 2017). A pro-inflammatory tumor microenvironment with heightened interferon response can promote anti-tumor immunity through attracting immune cells to the tumor site, promoting cytotoxic CD8 T cell priming by dendritic cells, boosting NK cell and CD8 T cell cytotoxicity, enhancing inflammatory cytokine production by macrophages and dampening regulatory T cell suppressive activity (Zitvogel et al., 2015). Thus, without being bound by a particular theory, PPARδ mediated tumor microenvironment remodeling can lead to tumor rejections through boosting pro-inflammatory immune cell recruitment and activity, as well as impairing anti-inflammatory immune cell function.

In order to test the clinical relevance of these findings, a human melanoma single cell RNA sequencing dataset was analyzed (see, e.g., Tirosh et al., 2016) (FIG. 2F). It was determined the expression of top 50 upregulated genes across all vav-cre+ VP16-PPARδ+ TILs (“PPAR induced”) was significantly correlated with the expression of genes involved in hallmark IFNγ response and hallmark inflammatory response in the human TILs. Similarly, PPAR induced gene expression in human TILs was correlated with “immune response” genes, which were the top upregulated genes in the dataset involved in interferon response, antigen presentation, chemoattraction and immune cell survival. In addition, the expression of genes involved in PPAR signaling pathway (see, e.g., Kanehisa and Goto, 2000) was significantly correlated with immune response in human TILs. These results suggest that activation of PPAR signaling in human TILs is positively correlated with a pro-inflammatory anti-tumor immune response and therapies that promote PPAR signaling in TILs have the potential to enhance anti-tumor immunity and cancer immunotherapy efficacy.

### Additional Materials and Methods

The following materials and methods were used to generate the data shown in the indicated figures.
FIGS. 20A-20H—scRNAseq of TILs from AKPS Tumors
Day-22 orthotopic AKPS tumors were excised and tumor infiltrating lymphocytes were isolated as described above. CD45+ cells were sorted using Sony SH800S cell sorter. 2-3 tumors per group were combined prior to library preparation. For the steady state experiment, naïve splenocytes were isolated and CD45+ cells were sorted using Sony SH800S cell sorter.
Single cell libraries were prepared using a 10× Genomics Chromium Controller (10× Genomics, 120223), the 10× Genomics Chromium Next GEM Single Cell 3′ Gene Expression kit (10× Genomics, 1000268), the Chromium Next GEM Single Cell 5′ Kit v2 (10× Genomics, 1000263), and TCR Amplification Kit (10× Genomics, 1000254) according to manufacturer's instructions at Cold Spring Harbor Laboratory Single Cell Biology Shared Resource. Cell suspensions were adjusted to target a yield of 8,000 cells per sample. cDNA and libraries were checked for quality on Agilent Bioanalyzer, quantified by KAPA qPCR, and sequenced on either NextSeq500 or NextSeq2000 (Illumina) instruments to an average depth of approximately 20,000 reads per cell. The Cellranger pipeline (v6.0.0 10× Genomics) was used to align FASTQs to the mouse reference genome (10× Genomics, gex-mm10-2020-A) and produce digital gene-cell counts matrices with default parameters.
Single cell datasets for each experiment were independently assessed for data quality following the guidelines described by (Love et al., Genome Biol. 2014 Dec. 5; 15(12):1-21; Wickham, Ggplot2: elegant graphicsfor data analysis. p212, Use R! (Springer, New York, 2009)). Cells with more than 10% mitochondrial transcripts as well as cells that had fewer than 250 feature counts or expressed fewer than 500 genes were removed. After quality control (QC), Seurat (v5.0.1) (Butler et al., Nature Biotechnology 2018 36:5. 2018 Apr. 2; 36(5):411-20) was used for normalization, graph-based clustering and differential expression analysis. Each dataset was normalized using SCTransform and the 5000 most variable genes were identified with SelectIntegrationFeatures. All conditions were integrated into a singular dataset via using the PrepSCTIntegration, FindIntegrationAnchors, and IntegrateData functions (Stuart et al., Cell. 2019 Jun. 13; 177(7):1888-1902.e21). MAGIC imputation was conducted on integrated data to impute missing values and account for technical noise (van Dijk et al., Cell. 2018 Jul. 26; 174(3):716-729.e27). RunPCA was implemented on the integrated datasets to identify the top 25 principal components (PCs) that were used for UMAP analysis and clustering. UMAP was calculated using the runUMAP function. Clustering was conducted by first constructing a nearest neighbor graph using the FindNeighbors function and then implementing the FindClusters function to perform clustering using the Louvain algorithm at a resolution of 1. Clusters were labeled in accordance with CD45 tumor infiltrating lymphocyte subtype signatures identified by (Zheng et al., Science (1979). 2021 Dec. 17; 374(6574)). Differential expression analysis was conducted between groups using the FindMarkers function with the MAST method to evaluate differences within the transcriptome (Finak et al., Genome Biol. 2015 Dec. 10; 16(1):1-13). T cell and B cell clonality was assessed using the scRepertoire package (v2.0.0 (Borcherding et al., F1000Res. 2020; 9)). Scores were assigned calculating the average z-score of the average expression of the genes in each cell. Wilcoxon rank-sum tests to determine if gene expression was significant was conducted using the wilcox.test function in stats (v4.1.0, (R: A Language and Environment for Statistical Computing. R Foundation for Statistical Computing, Vienna.; 2019. scirp.org/reference/referencespapers?referenceid=3131254)). Differential interactome modeling was conducted using the NicheNet (v2.0.5 (Browaeys et al., Nat Methods. 2020 Feb. 1; 17(2):159-62)) and CellChat packages (v2.1.0 (Jin et al., Nature Communications. 2021 Feb. 17; 12(1):1-20)). Gene set enrichment analysis was conducted using EnrichR (v3.2, (Kuleshov et al., Nucleic Acids Res. 2016 Jul. 8; 44(W1):W90-7)). Analysis code is available on GitHub (github.com/Vyoming/A-metabolic-switch-that-boosts-immune-cell-fitness-against-cancer.git).

### Example 3: Immune Cell Specific PPARδ Activation does not Lead to any Adverse Effects

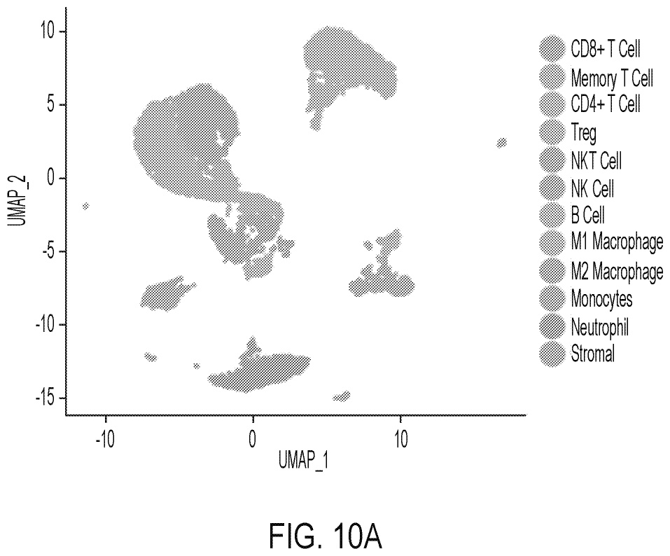

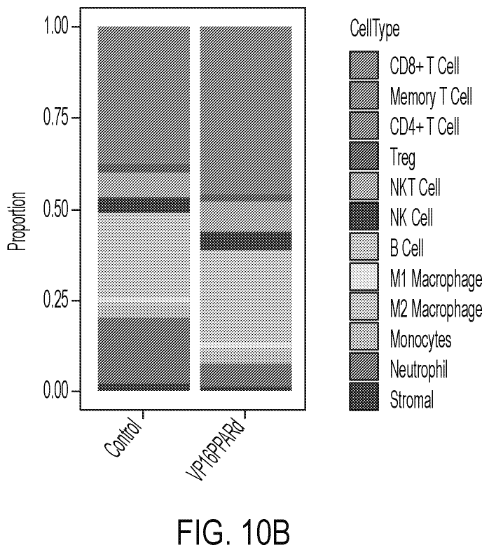

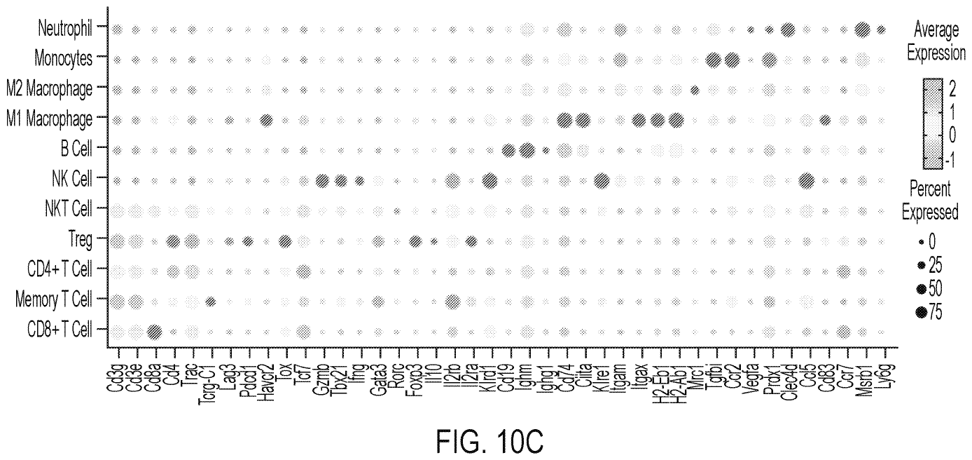

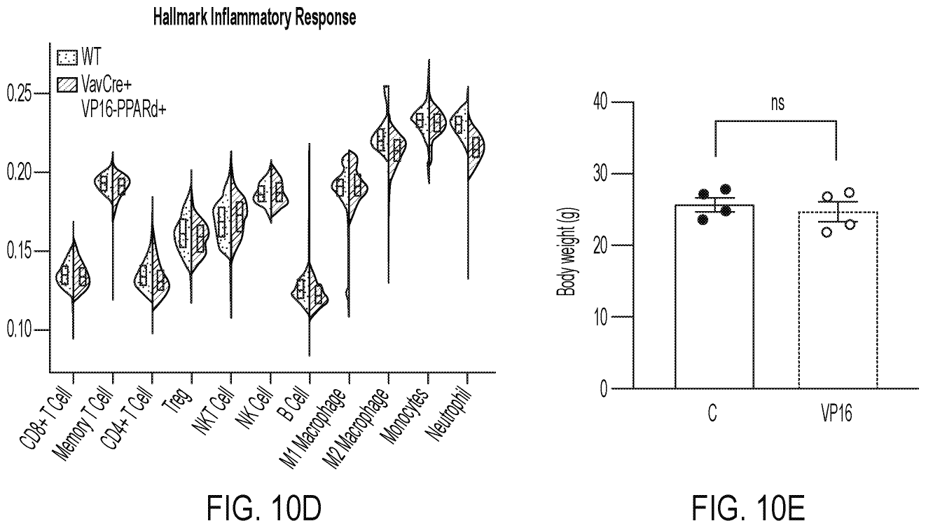

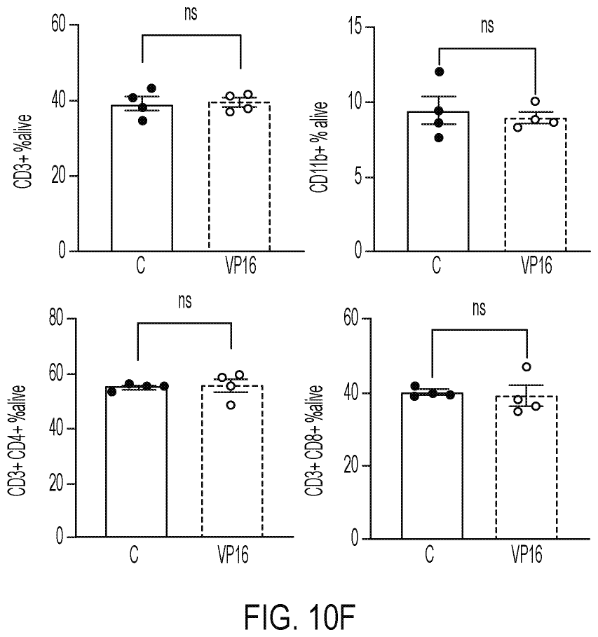

Cancer immunotherapies are designed to invigorate the immune system in order to eliminate malignant cells. However, unleashing the immune system against cancer cells often leads to adverse effects and toxicities such as cytokine release syndrome (CRS), immune effector cell-associated neurotoxicity syndrome (ICANS), or auto-immunity (Kennedy and Salama, 2020). Following the findings on PPARδ mediated pro-inflammatory response in tumor infiltrating immune cells disclosed above, the safety of vav-cre driven PPARδ gain-of-function was assessed. In order to assess the transcriptional changes upon PPARδ gain-of-function in immune cells at steady state, splenocytes were isolated from vav-cre+ VP16-PPARδ+ mice and single cell RNA sequencing was performed. The clustering analysis revealed major lymphoid and innate immune cell types (FIGS. 10A-10C). To determine if PPARδ signaling in immune cells lead to an inflammatory response prior to tumor challenge, the expression of hallmark inflammatory response genes across splenocytes was scored. In contrast to TILs, PPARδ activation in immune cells did not elevate the expression of genes involved in inflammatory response (FIG. 10D). This result is in line with the phenotypic observations, where vav-cre+ VP16-PPARδ+ mice do not exhibit any signs of disease, have comparable life-spans and similar body weights to age and sex matched control mice (FIG. 10E). In addition, flow cytometry analysis of splenocytes revealed comparable percentages of myeloid and lymphoid cells between vav-cre+ VP16-PPARδ+ and control mice (FIG. 10F). Moreover, histological examination of several tissues such as lung, spleen, liver, small intestine and colon by a board-certified pathologist confirmed the absence of any signs of auto-immunity (FIG. 10G). PPARδ gain-of-function did not cause any toxicity not only prior to tumor challenge, but also after tumor rejections. PPARδ activation in immune cells led to durable and safe anti-tumor responses, without any tumor relapses or signs of auto-immunity. Thus, PPARδ gain-of-function in immune cells is sufficient to promote anti-tumor immunity without causing any adverse effects. Developing novel therapeutics that activate PPARδ signaling in immune cells can lead to robust and safe alternatives to current cancer immunotherapies.

### Example 4: Cooperative Interactions Between Pro-Inflammatory Myeloid Cells and Cytotoxic CD8 T Cells Mediate PPARδ Driven Boost in Anti-Tumor Immunity

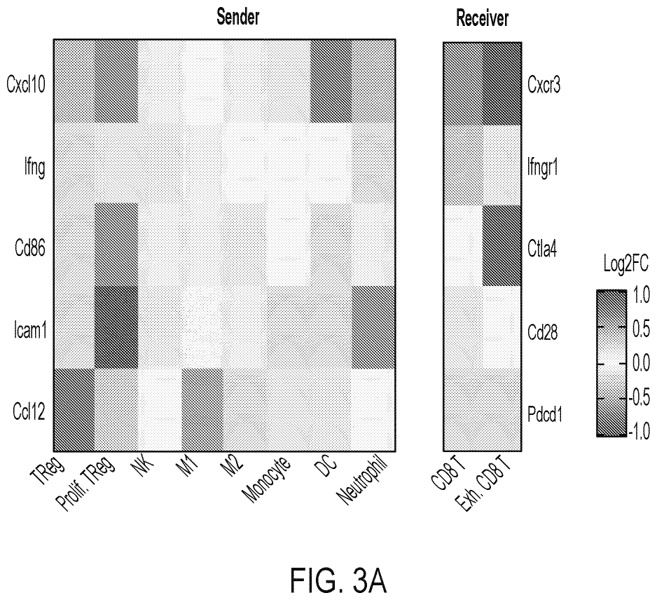

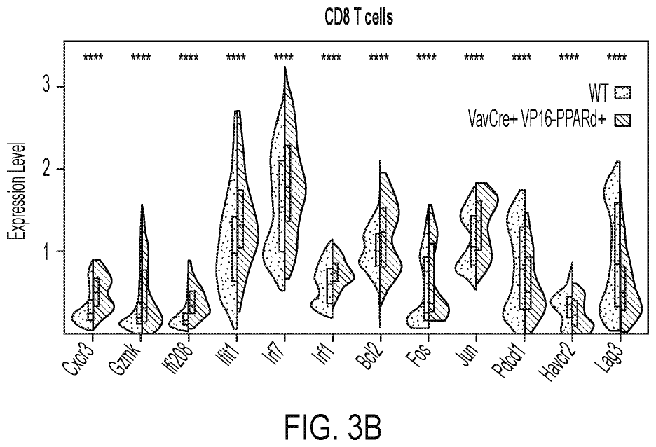

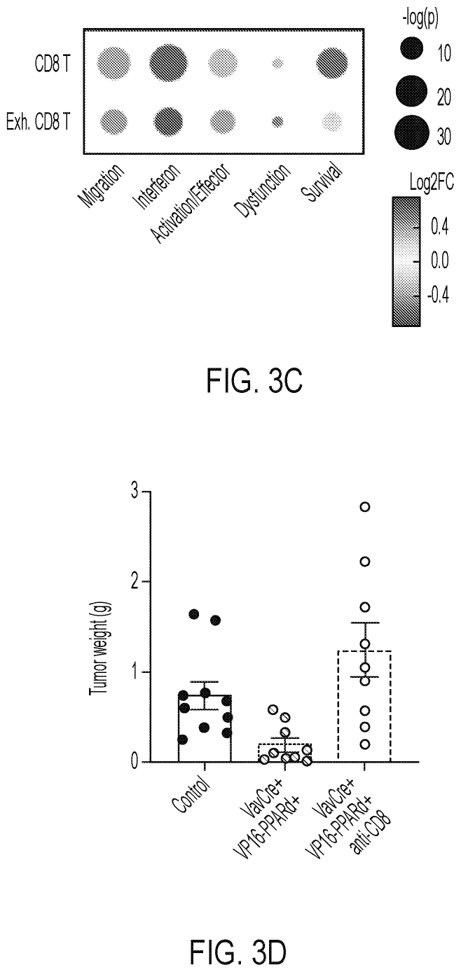

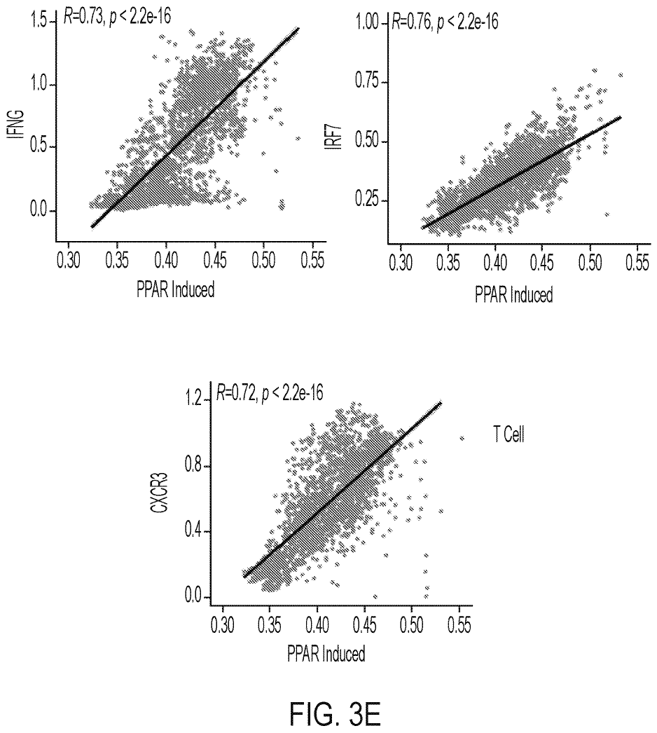

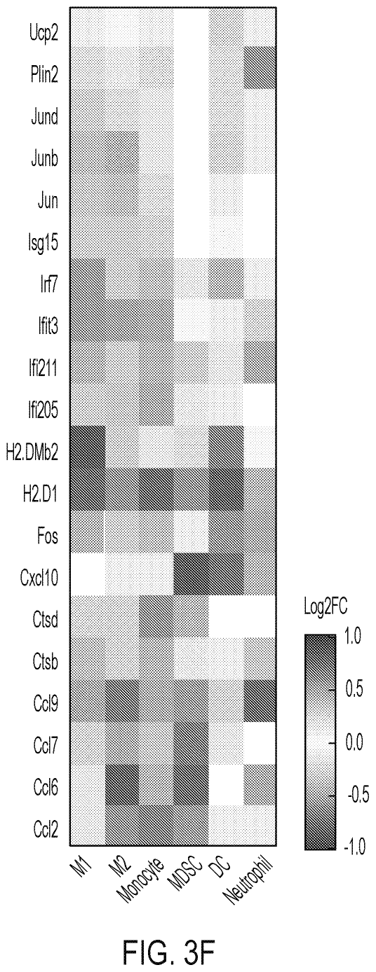

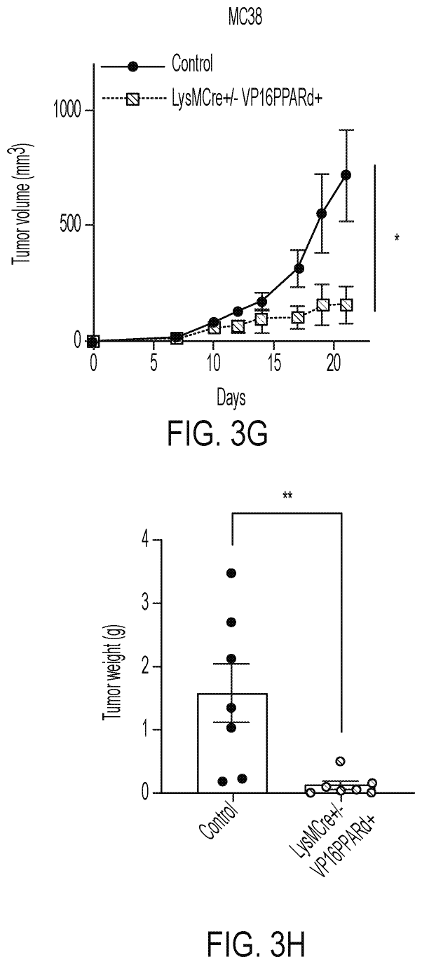

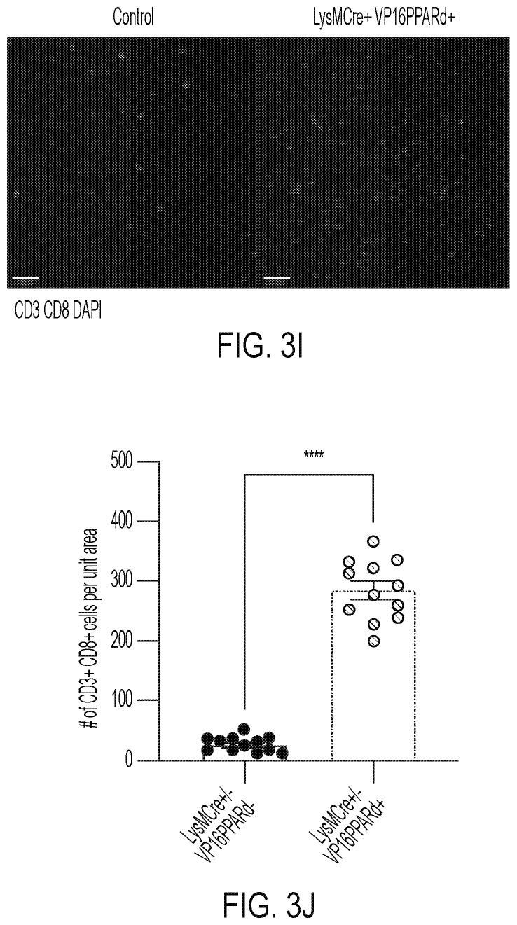

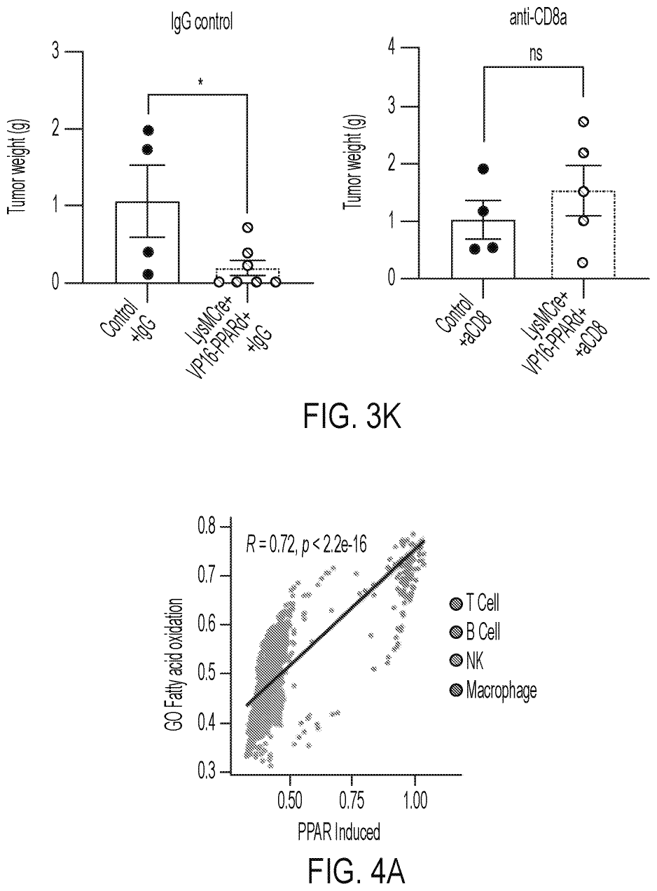

To understand cell-cell interactions in the tumor microenvironment that mediate PPARδ driven boost in anti-tumor immunity, the TIL scRNA-seq data was analyzed using a ligand-target prediction algorithm NicheNet (Browaeys et al., 2020). NicheNet analysis predicted up-regulation of CXCL10-CXCR3 axis in VP16-PPARδ+ TILs (FIG. 3A) CXCL10 is an interferon inducible chemokine mainly produced by myeloid cells such as dendritic cells, and its receptor CXCR3 is expressed on effector CD8 T cells and is essential for migration to inflammatory sites (Groom and Luster, 2011a). Expression of CXCR3 ligands positively correlates with PD-1 blockade immunotherapy response, and inducing CXCR3 ligands enhances anti-PD-1 immunotherapy response (Chow et al., 2019).

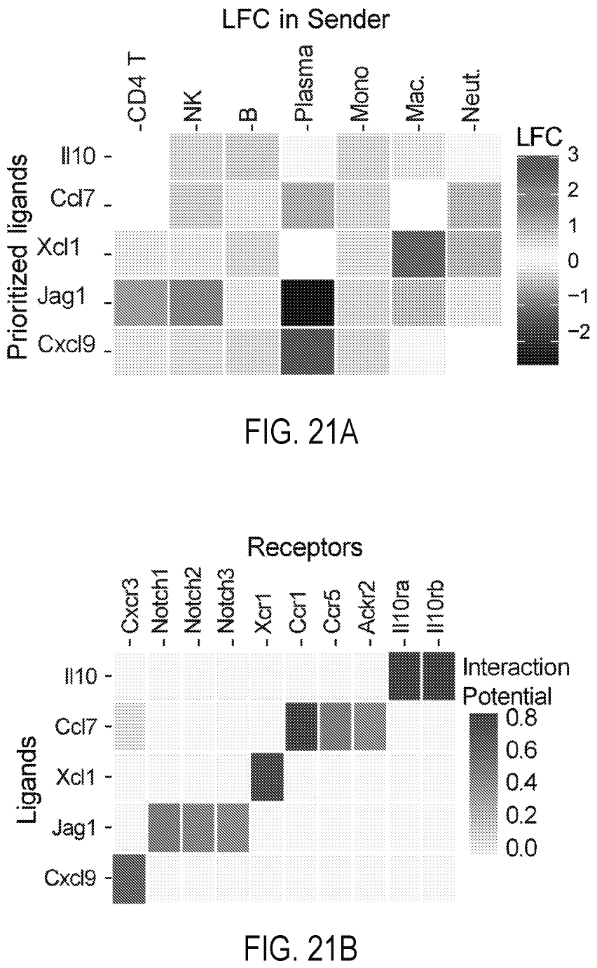

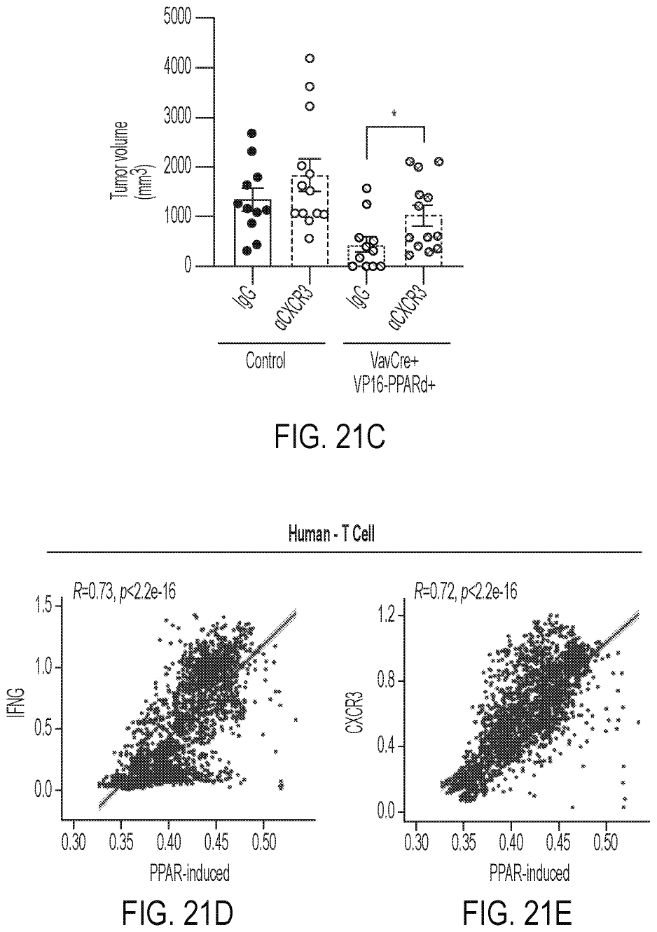

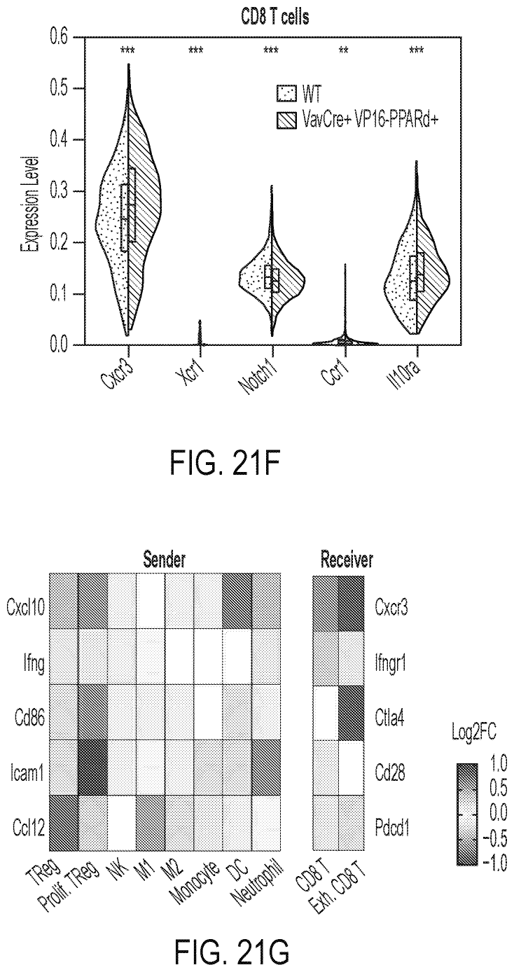

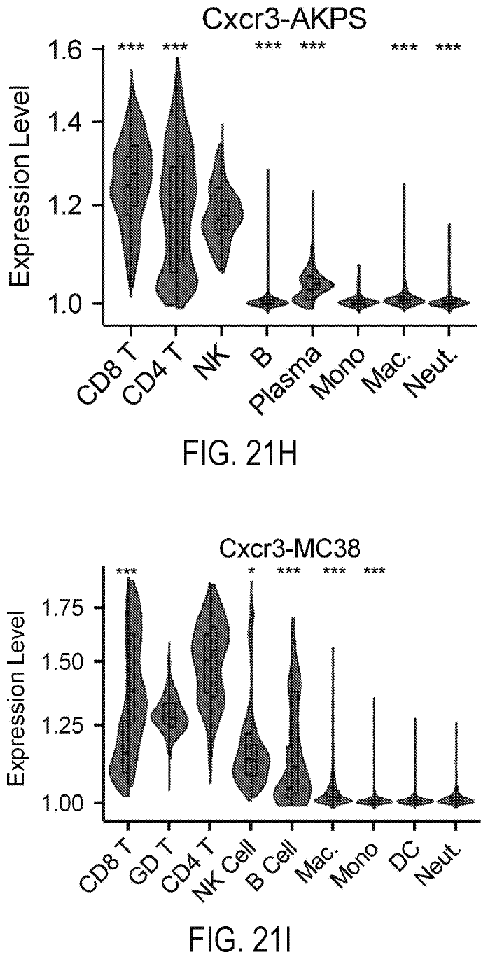

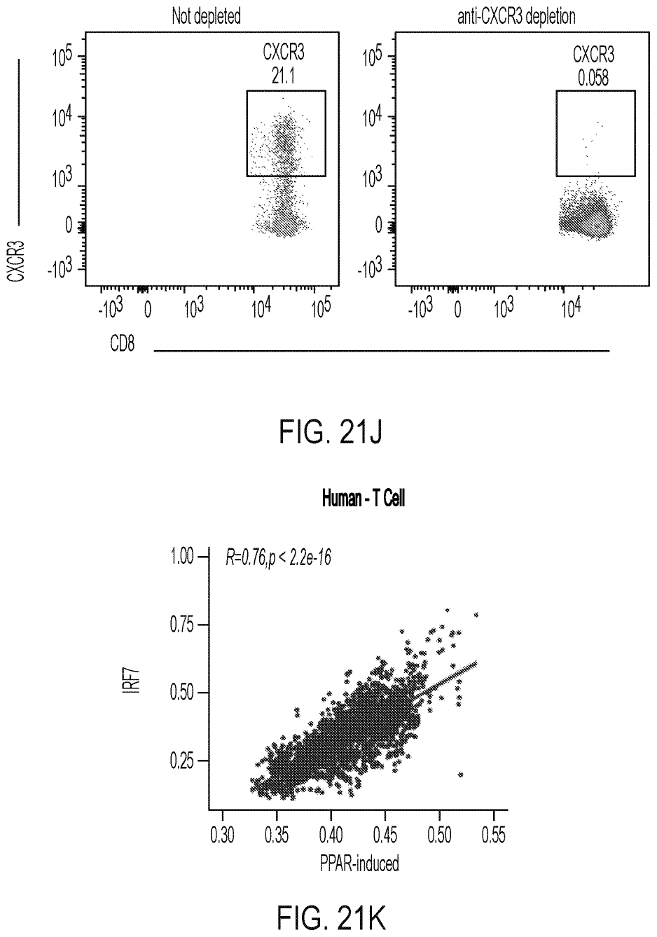

To understand cell-cell interactions in the tumor microenvironment that mediate PPARδ-driven boost in anti-tumor immunity, ligand-target prediction algorithm NicheNet (Sharma et al., Cell. 2017 Feb. 9; 168(4):707-72) was utilized. NicheNet analysis predicted upregulation of CXCL9-CXCR3 axis in AKPS VP16-PPARδ+ TILs and CXCL10-CXCR3 axis in MC38 VP16-PPARδ+ TILs (FIGS. 21A-21B and FIG. 21G). CXCL9 and CXCL10 are IFN-inducible chemokines, and their receptor CXCR3 is expressed on effector CD8 T cells and is essential for migration to inflammatory sites (Sterner et al., Blood Cancer J. 2021 Apr. 6; 11(4):6). Immune cell-intrinsic PPARδ activation significantly upregulated Cxcr3 expression in tumor infiltrating CD8 T cells (FIGS. 21F and 21H-21I) and depletion of Cxcr3+ cells significantly increased tumor size compared to IgG control (FIGS. 21C and 21J), demonstrating the necessity of Cxcr3+ cells for PPARδ-mediated anti-tumor immunity. Notably, expression CXCR3 and other functional markers in human melanoma infiltrating CD8 T-cells correlated with PPAR-induced gene signatures (FIGS. 21D-21E and 21K). These findings suggest a significant role for CXCR3 in CD8 T cells for PPARδ-induced anti-tumor effects.

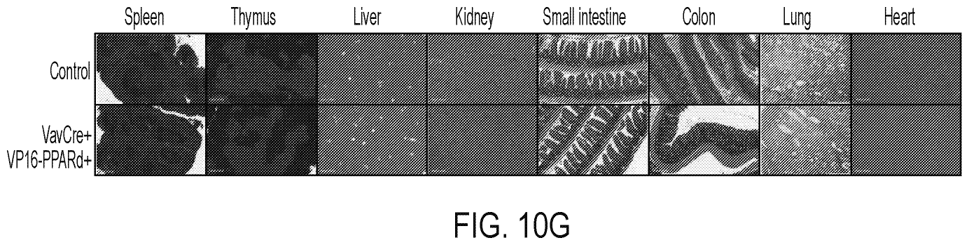

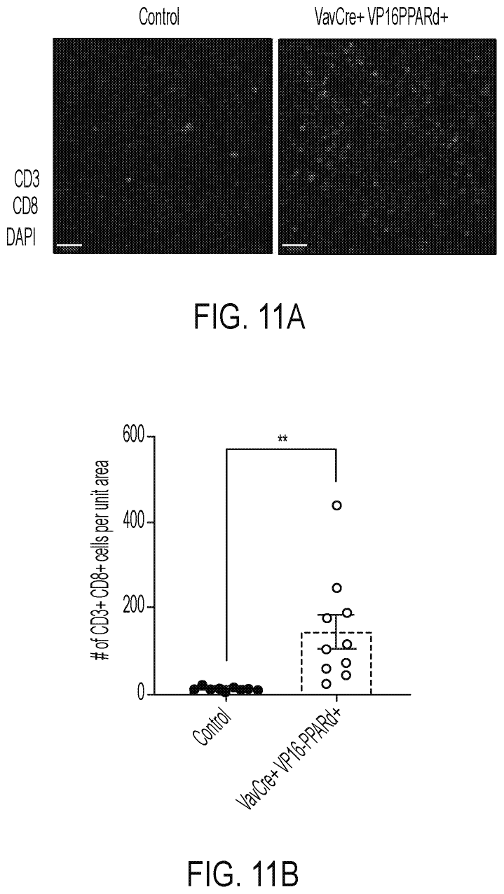

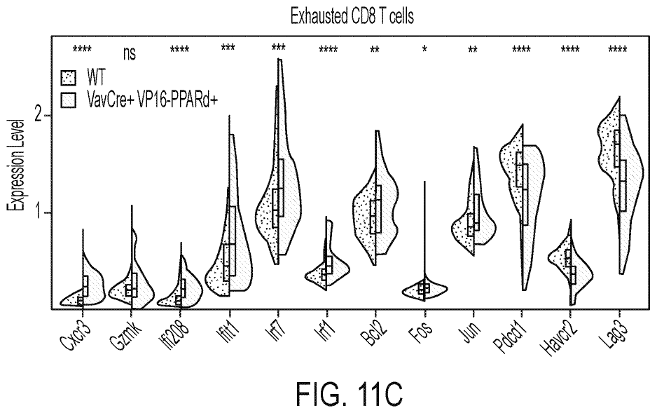

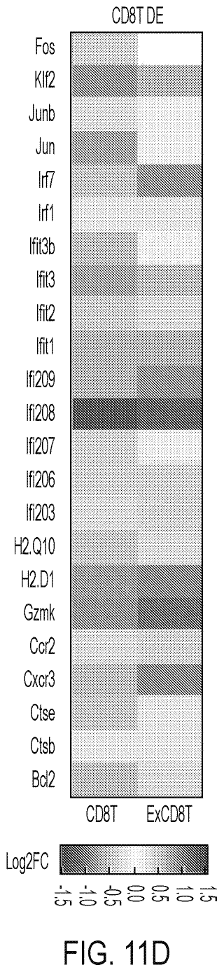

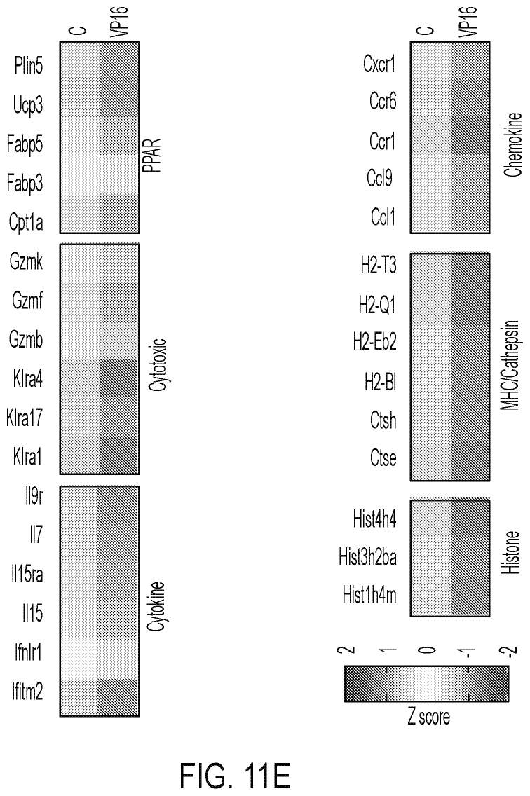

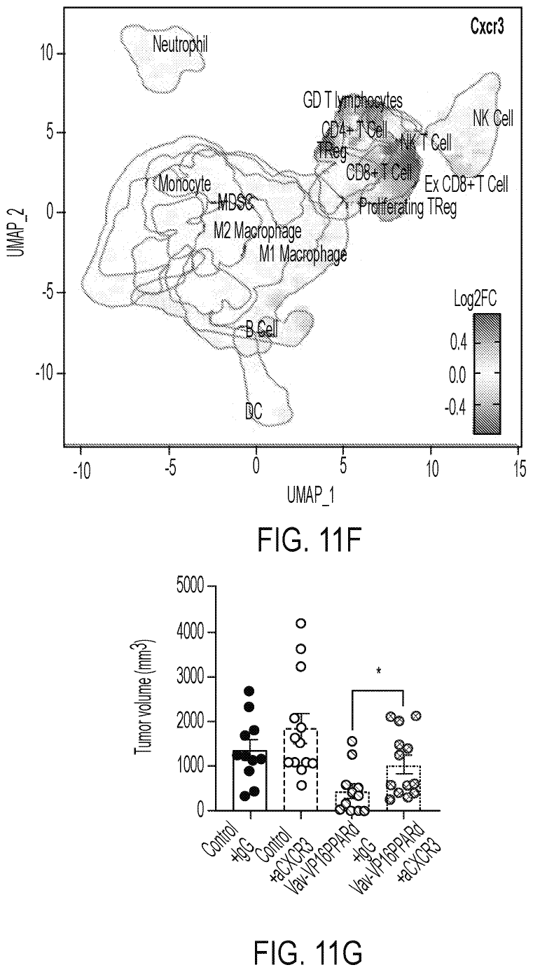

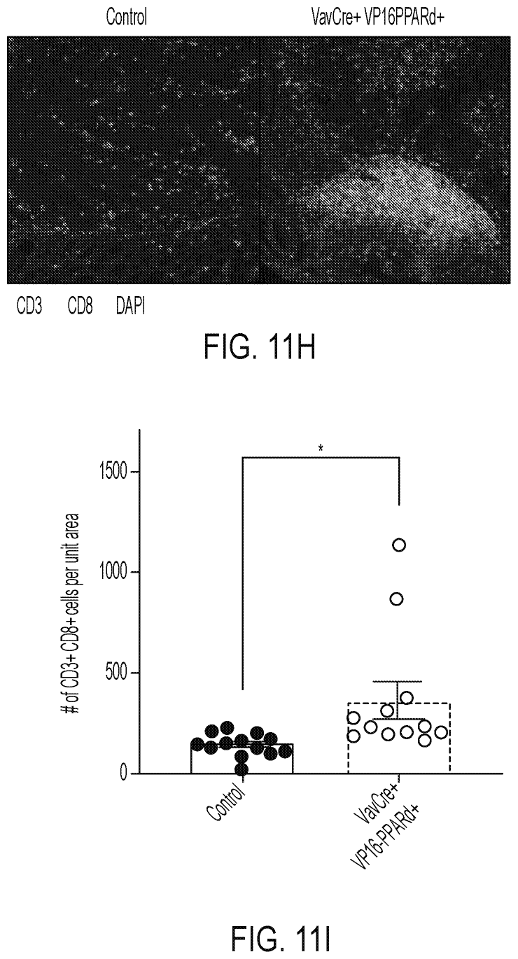

CD8 T cell presence was analyzed in MC38 tumors grown in vav-cre+ VP16-PPARδ+ and control mice using immunofluorescence staining. It was determined PPARδ gain-of-function in immune cells significantly increased the number of tumor-infiltrating CD8 T cells in subcutaneous MC38 tumors (FIGS. 11A and 11B). However, even tumors that are highly infiltrated by CD8 T cells continue to progress, which can indicate dampened effector function of tumor infiltrating CD8 T cells (Hellstrom et al., 1968; Philip and Schietinger, 2022). Tumor microenvironments often lead to CD8 T cell “dysfunction” (or “exhaustion”) marked by upregulation of inhibitory receptors, dampened cytokine secretion and impaired cytotoxic function (Schietinger et al., 2016; Speiser et al., 2016). Therefore, the transcriptional status of VP16-PPARδ+ tumor infiltrating CD8 T cells was assessed. Single cell RNA sequencing analysis of tumor-infiltrating VP16-PPARδ+ CD8 T cells revealed heightened expression of markers associated with CD8 T cell migration (Cxcr3), effector function (Gzmk), interferon response (Irf7, irf1) and survival (Bcl2, Fos, Jun), as well as dampened expression of T cell dysfunction markers PD-1 (Pdcd1), Tim-3(Havcr2) and Lag-3 (FIGS. 3B, 3C, and 11D). (Charo et al., 2005; Groom and Luster, 2011a; Honda et al., 2006; LaFleur et al., 2019; Philip and Schietinger, 2022; Shaulian and Karin, 2001). Surprisingly, PPARδ activation was also sufficient to promote the expression of genes involved in CD8 T cell effector function in exhausted CD8 T cells, a subset of CD8 T cells that express markers of CD8 T cell dysfunction such as transcription factor Tox, as well as inhibitory receptors PD-1, Tim-3 and Lag-3 (Scott et al., 2019). It is currently unclear whether dysfunctional T cells can be reinvigorated (Philip and Schietinger, 2022). Nevertheless, gene expression profile of vav-cre+ VP16-PPARδ+ exhausted CD8 T cells exhibit increased markers of T cell effector function (FIGS. 11C and 11D). Similarly, bulk RNA sequencing of CD8 T cells isolated from tumor draining lymph nodes revealed elevated expression of genes involved in cytotoxic function (Gzmb, Gzmf) and migration (Cxcr1, Ccr6) (FIG. 11E) (Kondo et al., 2007; Shi et al., 2009; Takata et al., 2004). In addition, several genes encoding for histone proteins (Hist4h4, Hist1h4m) were upregulated in vav-cre+ VP16-PPARδ+ CD8 T cells, which can indicate enhanced proliferation as histone synthesis is linked to cell cycle progression (see, e.g., Nelson et al., 2002). Taken together, these results demonstrate that PPARδ activation in immune cells promotes CD8 T cell tumor infiltration as well as migration, survival, and effector function, which leads to complete tumor elimination by the immune system.

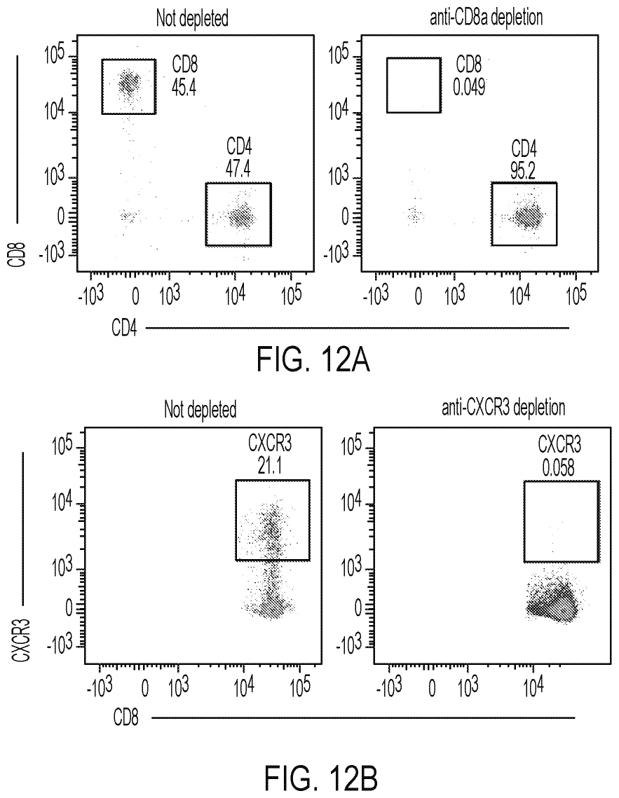

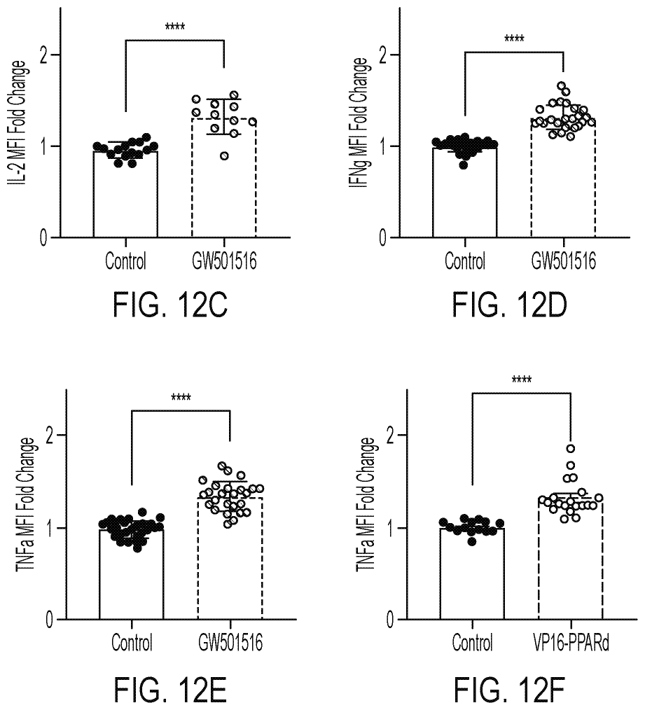

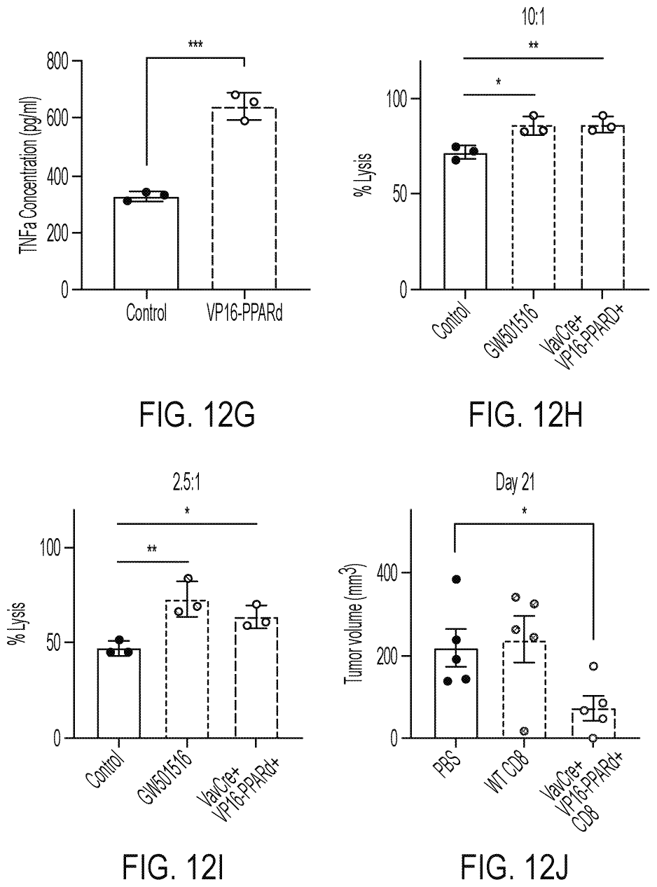

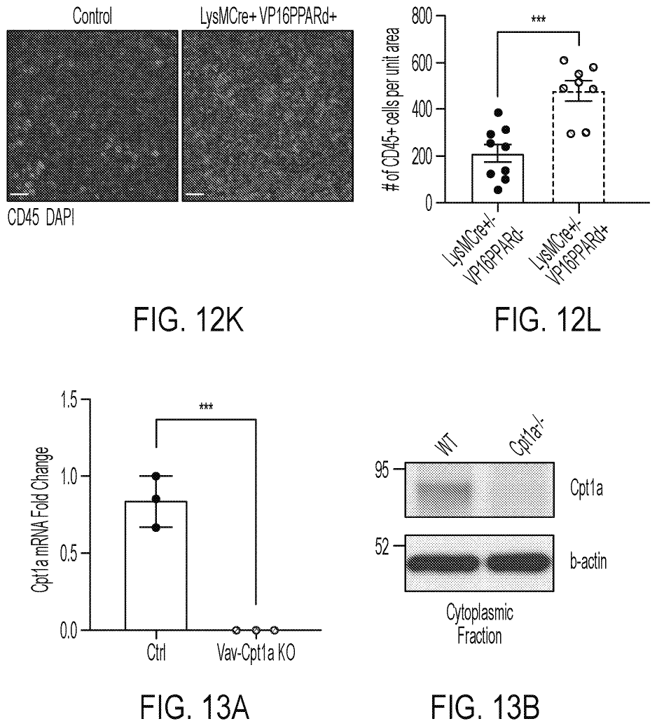

Next, in order to test the involvement of CD8 T cells for PPARδ driven anti-tumor immunity, CD8+ cells were depleted using in vivo depletion antibodies targeting the alpha subunit of CD8 (CD8α). A 100% CD8 T cell depletion efficiency was confirmed using peripheral blood flow cytometry analyses (FIG. 12). In the absence of CD8+ cells, PPARδ gain-of-function in immune cells failed to promote anti-tumor immunity against subcutaneous MC38 tumors, demonstrating the necessity of CD8+ cells for PPARδ mediated boost in anti-tumor immunity (FIG. 3D).

In order to investigate the CD8 T cell intrinsic effects of PPARδ activation, proxies of CD8 T cell function such as cytokine secretion and cytotoxicity upon genetic or pharmacological PPARδ activation in vitro were assessed. PPARδ agonist GW501516 treatment of murine CD8 T cells significantly increased the production of pro-survival cytokine IL-2, as well as pro-inflammatory cytokines IFNγ and TNFα (FIGS. 12C-12E). Similarly, CD8 T cells isolated from vav-cre+ VP16-PPARδ+ mice exhibited significantly increased TNFα secretion, measured by flow cytometry and ELISA (FIG. 12G). Moreover, GW501516-treated or VP16-PPARδ+ CD8 T cells demonstrated significantly enhanced in vitro cytotoxicity against MC38 cancer cell line (FIGS. 12H and 12I.). Thus, cell intrinsic pharmacological or genetic PPARδ activation promoted CD8 T cell effector function in vitro. Next, in order to study CD8 T cell sufficiency in vivo, VP16-PPARδ+ CD8 T cells were adoptively transferred to sub-lethally irradiated WT mice. After CD8 T cell adoptive transfer, MC38 cancer cells were subcutaneously injected. 21 days after cancer cell injection, mice that received VP16-PPARδ+ CD8 T cells had significantly smaller tumors compared to mice that received WT CD8 T cells (FIG. 12). Taken together, these results indicate that PPARδ activation in CD8 T cells is sufficient to enhance CD8 T cell effector function in vitro and in vivo.

Upon demonstrating the involvement CD8 T cells for PPARδ driven tumor rejections as well as the sufficiency of CD8 T cell intrinsic PPARδ activation for promoting CD8 T cell effector function, PPAR-induced genes were further examined to determine is the expression is associated with markers of enhanced T cell function in human TILs. Using a human melanoma TIL dataset (see, e.g., Tirosh et al., 2016), the correlation of PPAR-induced genes with key markers of T cell effector function (Ifng), interferon response (Irf7) and migration (Cxcr3) were determined. The expression of PPAR-induced genes was found to strongly correlate with Ifng, Irf7 and Cxcr3 expression in human tumor infiltrating T cells (FIG. 3E).

Similar to the correlation of Cxcr3 expression with PPAR-induced genes in human tumor infiltrating T cells, vav-cre driven PPARδ activation significantly upregulated Cxcr3 expression of tumor infiltrating T cells (FIG. 11F). Given the importance of Cxcr3 for T cell migration and function (Groom and Luster, 2011a), the involvement of Cxcr3+ cells for PPARδ mediated anti-tumor immunity was tested using in vivo depletion antibodies targeting Cxcr3 (FIG. 12B). Compared to IgG control treated vav-cre+ VP16-PPARδ+ mice, Cxcr3 depleted vav-cre+ VP16-PPARδ+ mice exhibited significantly increased tumor size (FIG. 11G). This indicates the relevance of Cxcr3 expressing immune cells (such as effector CD8 T cells and Th-1 polarized CD4 T cells) for PPARδ driven anti-tumor immunity. Surprisingly, PPARδ activation in immune cells was also sufficient to increase CD8 T cell infiltration into poorly immunogenic orthotopic AKP tumors (FIGS. 11H and 11I). In order to test if PPARδ activation and its downstream FAO pathways promote immune cell migration, human jurkat cells were treated with PPARδ agonist GW501516, or fatty acids (palmitic acid, oleic acid, arachidonic acid, or eicosapentaenoic acid) and performed an in vitro migration assay. Thus, without being bound by a particular theory, one of the mechanisms by which PPARδ enhances anti-tumor immunity could be promoting immune cell migration.

Innate and adaptive arms of the immune system work in synergy during robust inflammatory responses. Innate immune cells such as dendritic cells, macrophages and neutrophils are pivotal in priming adaptive immune responses that involve T and B cells (Iwasaki and Medzhitov, 2015). The outcome of an anti-tumor response is determined by the net balance of pro-inflammatory and anti-inflammatory factors. Therefore, it was assessed if PPARδ activation in innate immune cells could help alter this balance in favor of anti-tumor immunity. Consequently, the transcriptional status of tumor-infiltrating myeloid cells in MC38 tumors prior to tumor rejection was analyzed using scRNA-seq. Tumor-infiltrating myeloid cell populations are heterogeneous and plastic. Accumulating evidence suggests that tumor microenvironment skews myeloid cells towards tolerogenic, immune-suppressive states (Mantovani and Sica, 2010; Schouppe et al., 2012). Intriguingly, tumor-infiltrating myeloid cells from vav-cre+ VP16-PPARδ+ mice exhibited a robust pro-inflammatory, interferon-stimulated gene signature (Isg15, Irf7, Ifit3, Ifi208, Cxcl10) (FIG. 3F). (See, e.g., Fensterl and Sen, 2015; Groom and Luster, 2011b; Korant et al., 1984; Landolfo et al., 1998; Ning et al., 2011). Interferon signaling is crucial for anti-tumor immune responses and can regulate the activity of all immune cells. Interferons coordinate a complex anti-tumor immune response through dampening the suppressive function of myeloid derived suppressive cells (MDSCs), skewing macrophage polarization towards an M1-like immunostimulatory phenotype and promoting dendritic cell antigen presentation to T cells (Parker et al., 2016; Zitvogel et al., 2015). Thus, upregulation of an interferon response signature in tumor-infiltrating myeloid cells upon PPARδ gain-of-function suggests a role for myeloid cells in priming adaptive anti-tumor immunity.

To determine if PPARδ activation in myeloid cells is sufficient to promote anti-tumor immunity, LysMcre+ VP16-PPARδ+ mice were generated that overexpress the PPARδ gain-of-function fusion protein VP16-PPARδ+ in monocytes, macrophages and granulocytes (see, e.g., Clausen et al., 1999). When challenged with subcutaneous MC38 tumors, PPARδ gain-of-function in myeloid cells was sufficient to enhance anti-tumor immunity (FIG. 3G, 3H) Immunofluorescence analyses of MC38 tumors showed significantly increased CD45+ immune cell and CD8 T cell infiltration upon LysMcre driven PPARδ activation. (FIGS. 31, 3J, 12K, and 12L). This result was in line with upregulation of genes encoding pro-inflammatory chemokines such as Cxcl10, Ccl2, and Ccl3 in tumor-infiltrating myeloid cells in vav-cre+ VP16-PPARδ+ mice (FIG. 3F). Pro-inflammatory chemokines are not only crucial for attracting immune cells to sites of immune response, but they also regulate immune cell positioning and function (Sokol and Luster, 2015). To test if myeloid specific PPARδ gain-of-function promotes anti-tumor immunity through recruitment and activation of CD8 T cells, CD8+ cells in LysMcre+ VP16-PPARδ+ mice were depleted using in vivo depletion antibodies and assessed anti-tumor immune response against subcutaneous MC38 tumors. It was determined that PPARδ activation in myeloid cells failed to promote anti-tumor immunity in the absence of CD8+ cells (FIG. 3K). Thus, PPARδ activation in myeloid cells was sufficient to promote anti-tumor immunity in a CD8+ cell dependent manner.

### Additional Materials and Methods

The following materials and methods were used to generate the data shown in the indicated figures.

### FIGS. 21A-21B, 21G—Interactome Modeling

Differential interactome modeling was conducted using the NicheNet (v2.0.5), as described above. We plotted a summary of differential expression of top prioritized ligands and ligand-target predicted interaction potential between VP16-PPARδ+ tumor infiltrating immune cells in AKPS tumors as identified by NicheNet analysis.

### FIGS. 21F, 21H, and 21I—CXCR3 Expression

Differential expression analysis was conducted as described above.

### FIGS. 21C, 21J—CXCR3 Antibody MC38 Challenge

Mice were intraperitoneally injected with 200 ug in vivo depletion antibodies anti-CXCR3 (Bioxcell, clone CXCR3-17) or Armenian hamster IgG isotype control (Bioxcell, polyclonal) six times, on days −1, 1, 5, 9, 13, and 16, relative to subcutaneous tumor injection (day 0). For subcutaneous injection, mice were anesthetized using isoflurane, shaved at the injection site, and injected subcutaneously in the right flank with 3×10⁵ MC38 in 100 ul PBS. Tumor sizes were measured with a caliper every 2-3 days, until humane endpoints. Tumor volume was calculated with the formula 0.5×L×W² where L is the long diameter and W is the short diameter. Depletion efficiency was confirmed with tail bleeds followed by flow cytometry.

### FIGS. 21D-21E, 21K—CXCR3 in Human TIL Data

Single cell RNA sequencing data of melanoma samples from 19 patients was evaluated to determine if there was an association between PPAR associated responses and lymphocyte infiltration (Tirosh et al., Science (1979). 2016 Apr. 8; 352(6282):189-96). Scores were calculated using method detailed previously. Pearson correlation coefficient test was calculated to evaluate the significance of the association. Analysis code is available on GitHub (github.com/Vyoming/A-metabolic-switch-that-boosts-immune-cell-fitness-against-cancer.git).

### Example 5: PPARδ Target Cpt1a in Part Mediates the Effects of PPARδ on Anti-Tumor Immunity

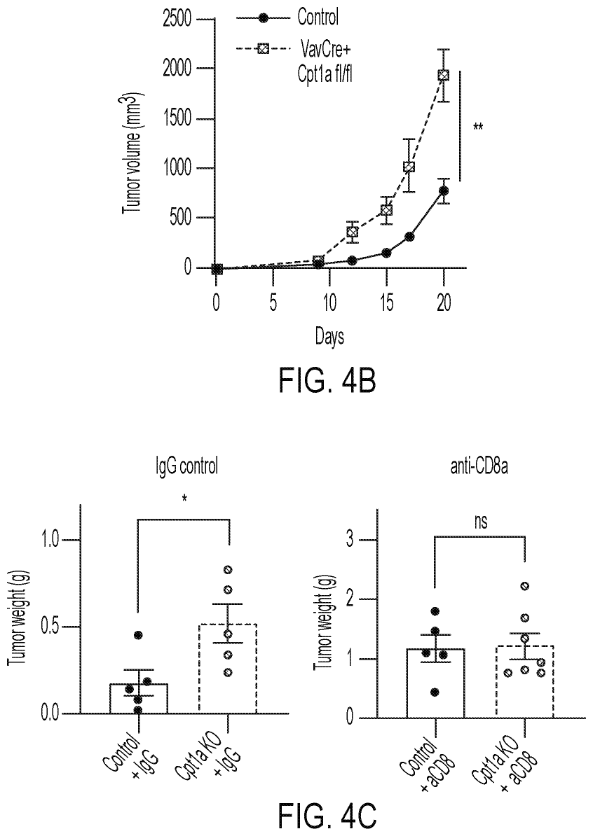

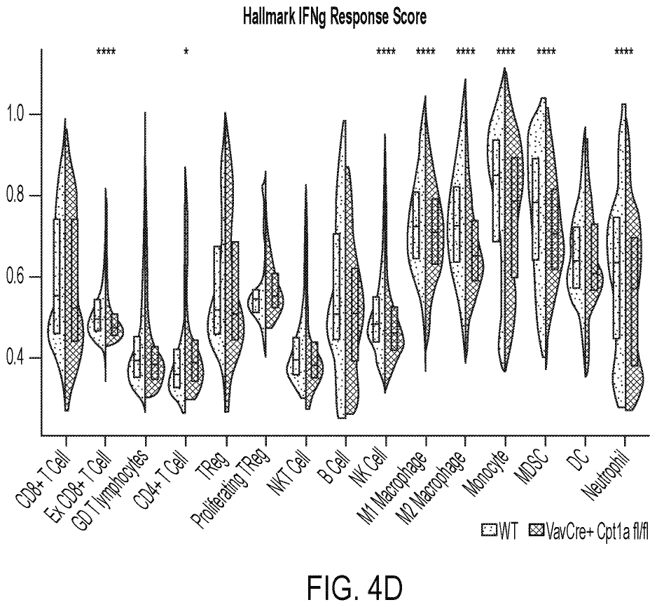

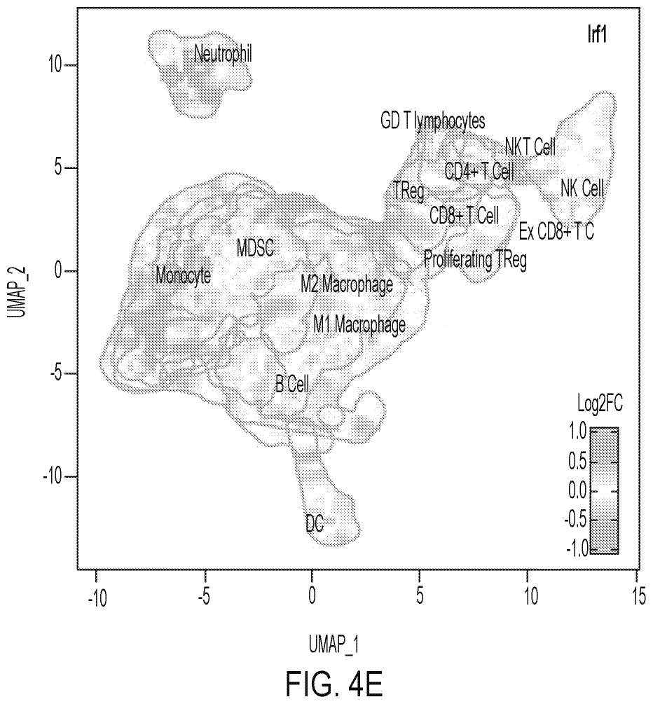

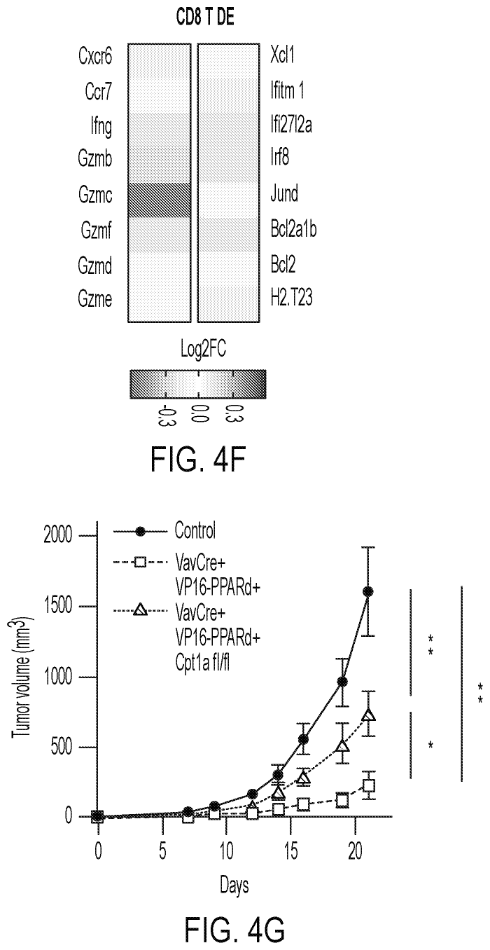

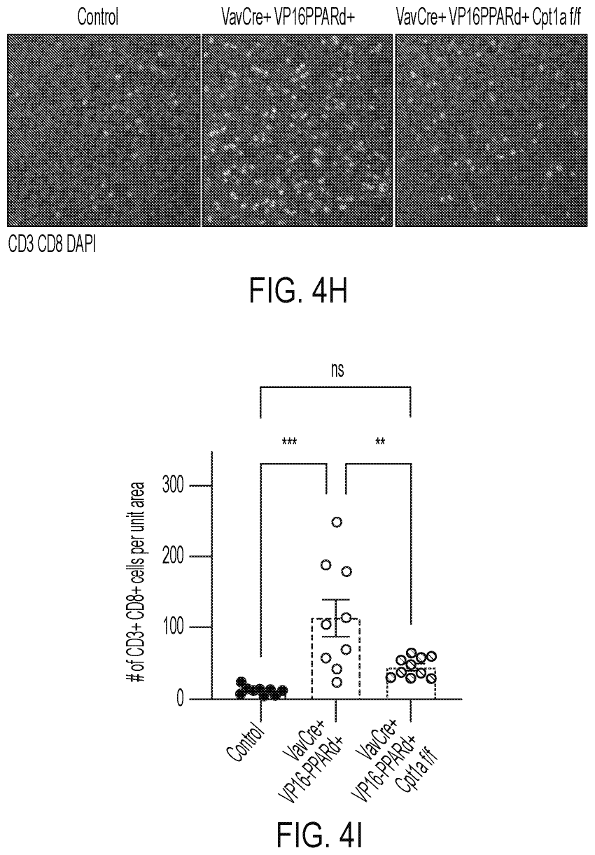

PPAR-induced genes from the TIL scRNA-seq experiment was strongly correlated with the expression of genes involved in fatty acid β-oxidation in human melanoma TILs (Tirosh et al., 2016) (FIG. 4A). The rate limiting enzyme of mitochondrial fatty acid β-oxidation (FAO) is carnitine palmitoyltransferase 1 (Cpt1). Cpt1a is the primary Cpt isoform that is expressed in various tissues such as liver, intestines and spleen, and is a key PPARδ target gene (Wang et al., 2004).

There are contradictory results on the effects of PPARδ-Cpt1a-FAO axis on immune cell function. Initial studies reported that pharmacological PPARδ activation or Cpt1a upregulation exert anti-inflammatory effects in murine auto-immunity models. In contrast, several studies reported pro-inflammatory effects of PPARδ-Cpt1a axis, exacerbating auto-immunity (Liu et al., 2018; Lochner et al., 2015). Recently, PPARδ mediated metabolic rewiring was shown to promote anti-tumor immunity and improve immunotherapy response (Chowdhury et al., 2018; Saibil et al., 2019; Zhang et al., 2017). However, PPARδ activation was also shown to support tumor suppressive functions of T_{regs} (Wang et al., 2020). In addition to these contrasting results, Raud et al. reported that PPARδ target Cpt1a mediated FAO is dispensable for T cell responses (Raud et al., 2018). Without being bound by a particular theory, a possible explanation for this discrepancy is the reliance on of pharmacological modulators due to the lack of immune lineage specific GEMMs. In order to address this gap in the field and to study the functional significance of PPARδ-Cpt1a axis in immune cells, a vav-cre driven Cpt1a loss-of-function mouse model (vav-cre+ Cpt1a^{fl/fl}) was generated.

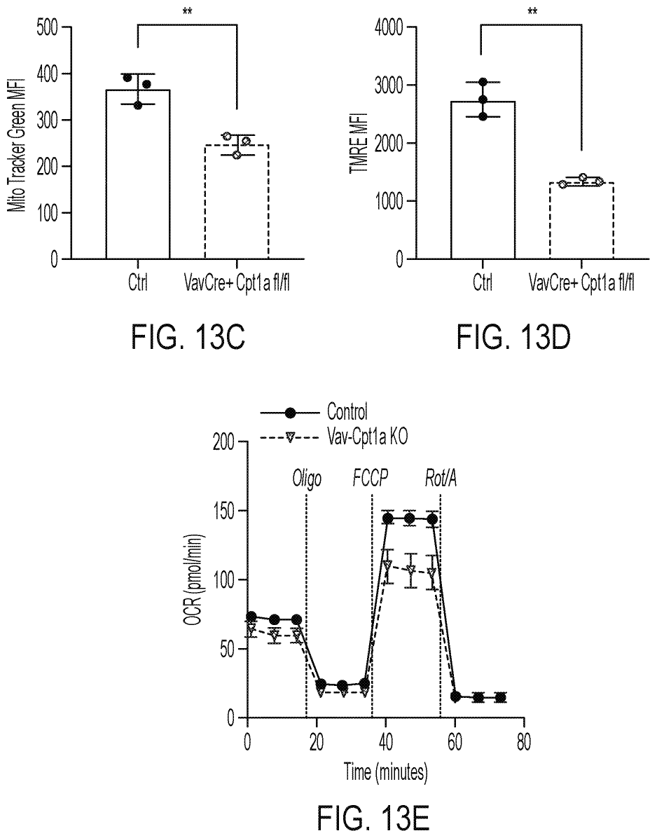

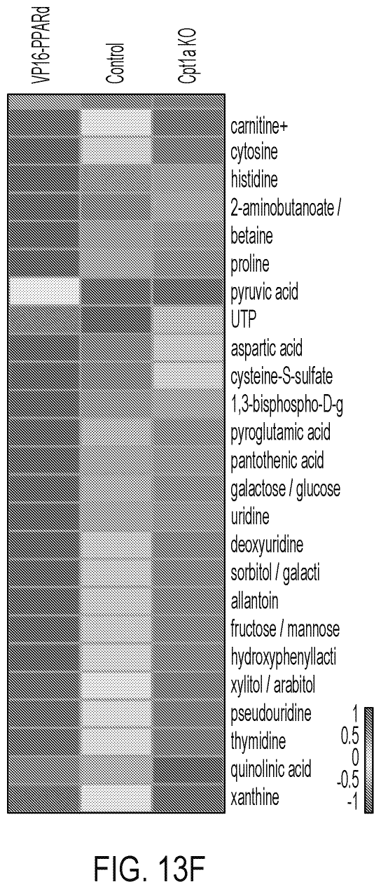

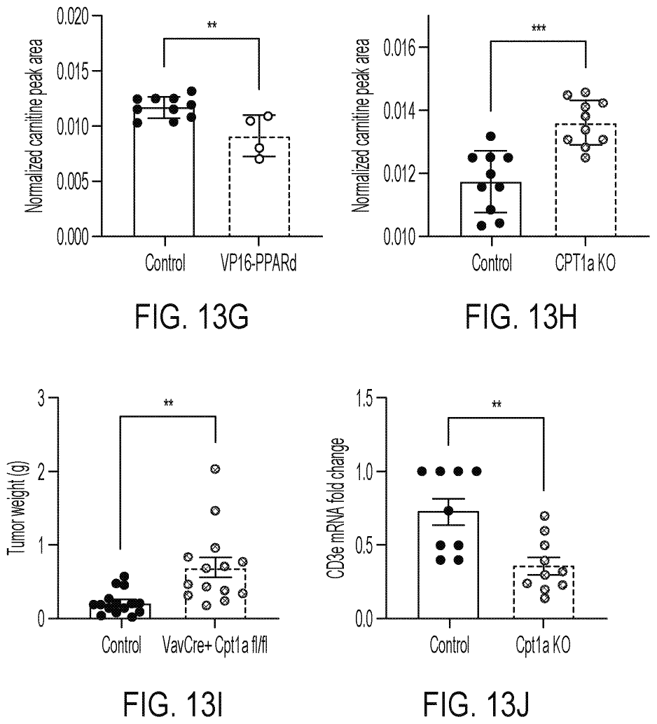

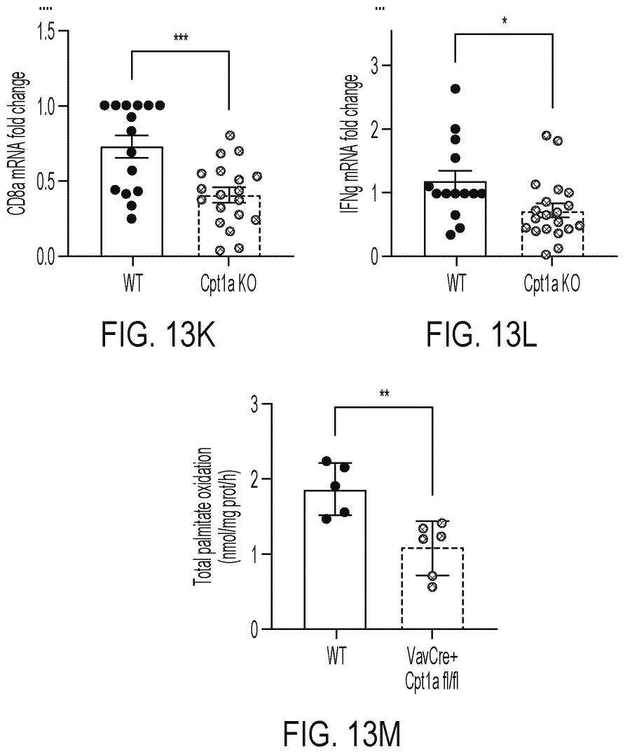

After confirming successful Cpt1a knock-out (KO) in immune cells at mRNA and protein levels (FIGS. 13A and 13B), the metabolic status of Cpt1a KO CD8 T cells was characterized using fluorescent dyes that localize to mitochondria. Fluorescent reporters that localize to mitochondria regardless of membrane potential (such as MITOTRACKER™ Green) are proxies of mitochondrial mass, while dyes that stain mitochondria depending on mitochondrial membrane potential [such as tetramethylrhodamine ethyl ester (TMRE)] indicate mitochondrial activity (Gokerkucuk et al., 2020). Accordingly, naïve CD8 T cells isolated from vav-cre+ Cpt1a^{fl/fl} mice were stained with MITOTRACKER™ Green and TMRE fluorescent dyes. It was determined that Cpt1a KO significantly dampens mitochondrial mass and activity, as measured by MITOTRACKER™ Green and TMRE fluorescent intensity respectively (FIGS. 13C and 13D). In addition, oxygen consumption rate (OCR) of Cpt1a KO CD8 T cells was measured using an extracellular flux analyzer. OCR is often used as a proxy of mitochondrial respiration (see, e.g., Voss et al., 2021). Extracellular flux analyzers can measure mitochondrial fitness by injecting various mitochondrial inhibitors and measuring OCR in real time. Extracellular flux analysis of Cpt1a KO CD8 T cells exhibited dampened OCR compared to control during maximal respiration, indicating reduced mitochondrial activity (FIG. 13E).

In order to further characterize the metabolic status of Cpt1a KO and VP16-PPARδ+CD8 T cells, polar metabolite levels were analyzed using liquid chromatography-mass spectrometry (LC-MS) (FIG. 13F). Cpt1a enzyme imports fatty-acyl-CoA molecules into the mitochondria in a carnitine depending manner (Schlaepfer and Joshi, 2020). Accordingly, it was determined that carnitine levels were decreased in VP16-PPARδ+ CD8 T cells compared to control, which can indicate carnitine being used by PPARδ target Cpt1a activity (FIG. 13G). Moreover, Cpt1a KO CD8 T cells had increased carnitine levels compared to control, where carnitine accumulation can be explained by impaired Cpt1a activity (FIG. 13H). In addition, levels of several metabolites involved in nucleotide metabolism (such as aspartate, uridine, thymidine) were increased in VP16-PPARδ+ CD8 T cells, while decreased in Cpt1a KO CD8 T cells (FIG. 13F) (see, e.g., Lane and Fan, 2015). Importantly, significantly decreased levels of ¹⁴C-palmitate oxidation were confirmed in Cpt1a KO CD8 T cells compared to WT CD8 T cells (FIG. 13M).

In order to study the necessity of Cpt1a mediated mitochondrial fatty acid β-oxidation in immune cells for anti-tumor immunity, vav-cre+ Cpt1a^{fl/fl} mice with subcutaneous MC38 tumors were challenged. In contrast to vav-cre+ VP16-PPARδ+ mice, Cpt1a KO in immune cells significantly increased MC38 tumor sizes compared to control (FIGS. 4B and 8I). This suggests that Cpt1a mediated fatty acid metabolism in immune cells helps mediate successful anti-tumor immune responses. In order to assess the proxies of T cell presence and function in MC38 tumors grown in vav-cre+ Cpt1a KO mice, RNA was isolated from tumor samples and quantified the expression of CD3e (T cell marker), CD8a (cytotoxic CD8 T cell marker) and IFNg (cytotoxic cytokine secreted by CD8 T cells and NK cells) genes using reverse-transcription quantitative PCR (RT-qPCR). It was determined that mRNA levels of CD3e, CD8a and IFNg genes were significantly dampened in MC38 tumors that were grown in vav-cre+ Cpt1a KO mice (FIGS. 13J-13L). This result suggests decreased presence and effector function of CD8 T cells upon Cpt1a KO. Accordingly, the involvement of CD8+ cells for impaired anti-tumor immunity upon Cpt1a loss in immune cells was tested using in vivo depletion antibodies. MC38 tumor sizes were comparable between control and Cpt1a KO mice upon CD8+ cell depletion, demonstrating the necessity of CD8+ cells for the impaired anti-tumor immunity phenotype observed in vav-cre+ Cpt1a KO mice (FIG. 4C).

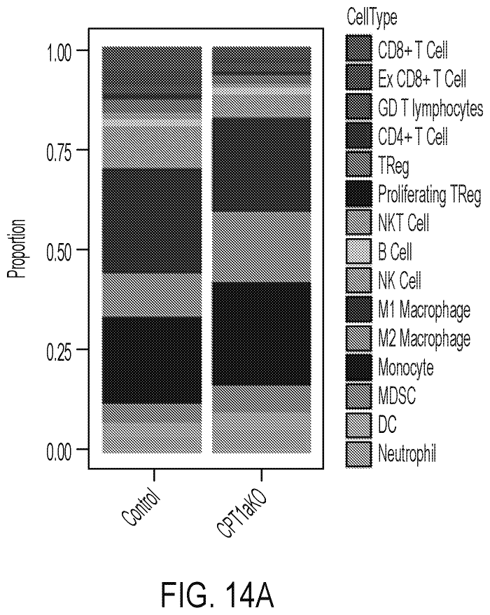

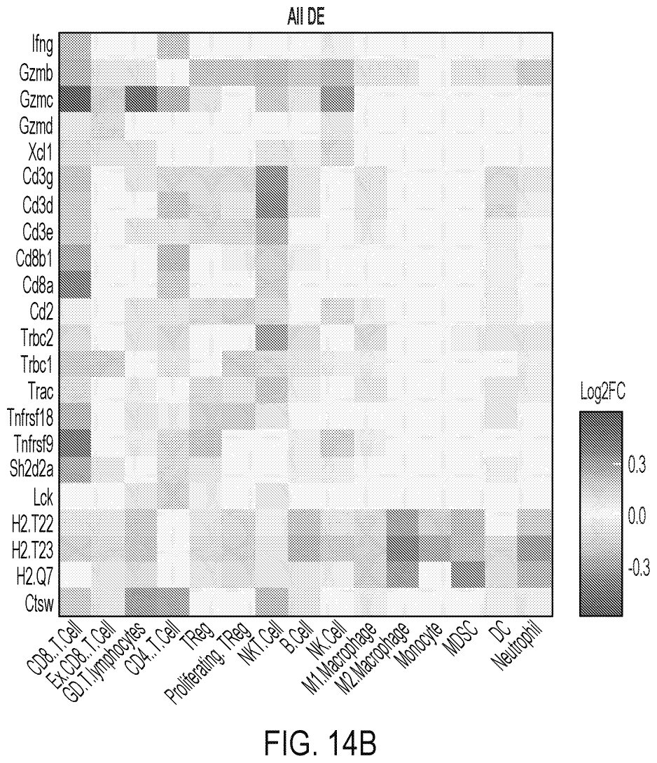

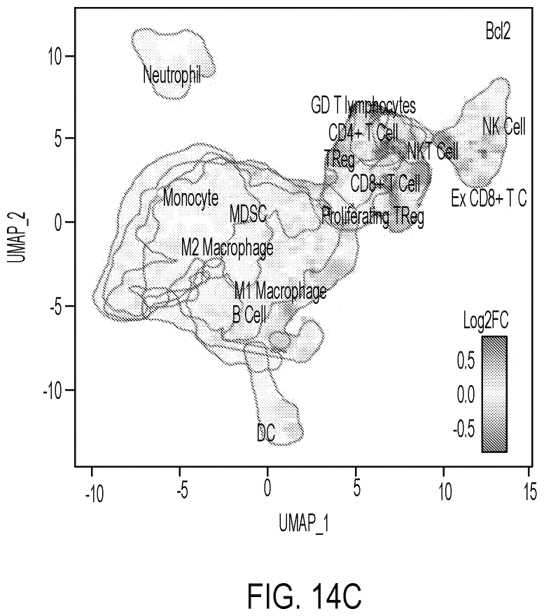

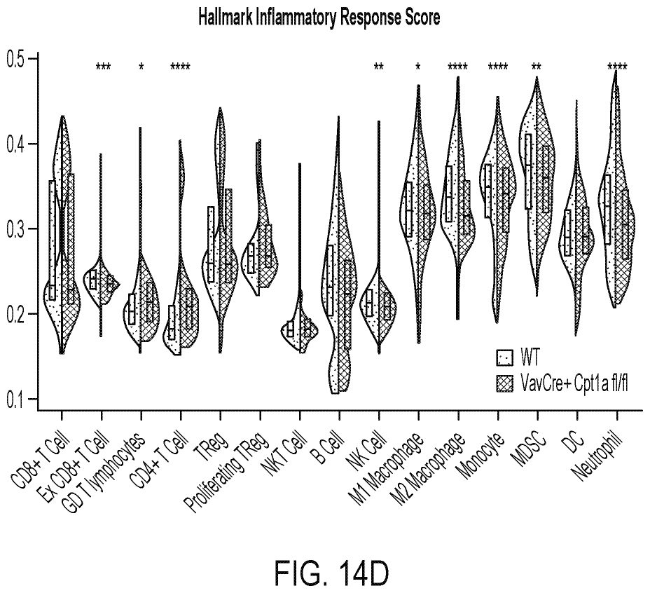

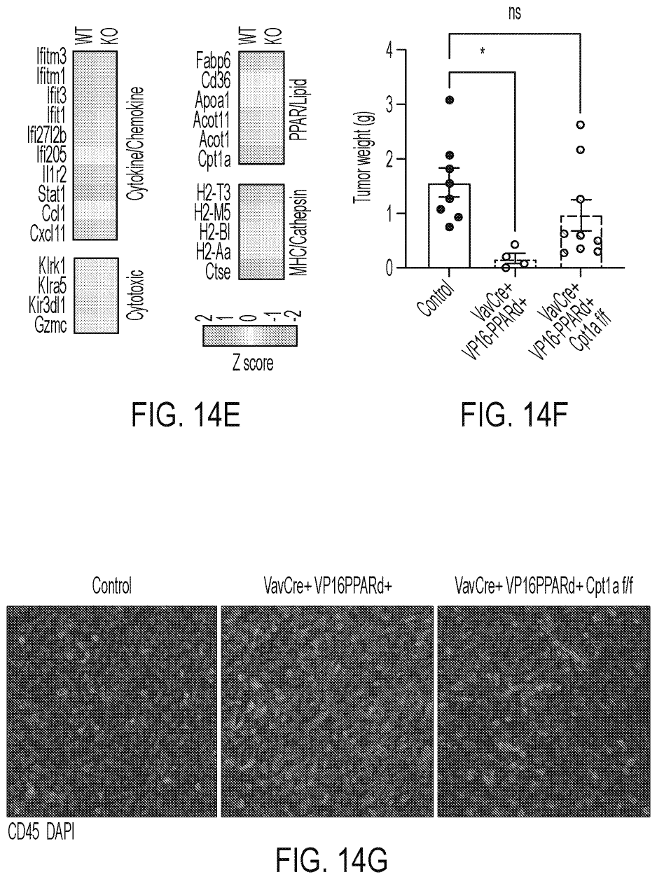

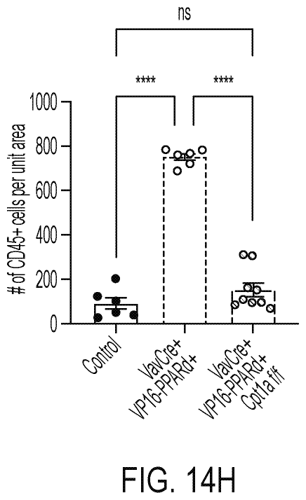

Next, the transcriptional changes in tumor infiltrating immune cells upon Cpt1a loss was assessed. MC38 tumor-infiltrating CD45+ TILs were isolated 12 days after subcutaneous tumor injection and performed scRNA-seq. Overall, Cpt1a KO TILs exhibited significant differences compared to VP16-PPARδ+ TILs. First, Cpt1a KO TILs exhibited dampened expression of MHC molecules H2-T22, H2-T23, H2-Q7, as well as cytotoxic molecules Ifng, Gzmb, Gzmc (FIG. 14B) (Getachew et al., 2008). Second, expressions of hallmark IFNg response and hallmark inflammatory response genes were decreased across several tumor infiltrating immune cell types (FIGS. 4D and 14D). Third, the expression of Irf1, a key regulator of type I IFN responses, was diminished across TILs (FIG. 4E). Moreover, tumor infiltrating CD8 T cells had decreased expression of genes involved in migration (Cxcr6, Cxcr7), cytotoxic function (Ifng, Gzmb, Gzmc), interferon response (Ifitm1, Irf8) and survival (Jund, Bcl2) (FIG. 4F). In addition, the expression of pro-survival protein Bcl2 was significantly diminished in several T cell clusters (FIG. 14C). Furthermore, bulk RNA-seq of CD8 T cells isolated from tumor draining lymph nodes corroborated the alterations in TIL scRNAseq, and revealed dampened expression of genes involved in interferon response (Ifit1, Ifit3) and cytotoxic function (Gzmc), as well as lipid metabolism (Fabp6, Cd36) (FIG. 14E) (Chabowski et al., 2007). Taken together, these results suggest that Cpt1a mediated mitochondrial fatty acid β-oxidation in immune cells is required for anti-tumor immunity. In contrast to pro-inflammatory TME rewiring upon PPARδ activation in immune cells, Cpt1a loss in TILs leads to dampened inflammatory response, along with decreased CD8 T cell migration, effector function and survival.

Upon demonstrating the opposing effects of PPARδ activation and deletion of PPARδ target gene Cpt1a in immune cells, the role of Cpt1a mediated mitochondrial fatty acid (3-oxidation was assessed in PPARδ driven boost in anti-tumor immunity. Accordingly, a mouse model was generated that over-expressed the constitutively active PPARδ fusion protein VP16-PPARδ in their immune cells, but lacked the rate limiting enzyme of mitochondrial fatty acid β-oxidation Cpt1a (vav-cre+ VP16-PPARδ+ Cpt1a^{fl/fl}). When challenged with subcutaneous MC38 tumors, VP16-PPARδ+ Cpt1a KO mice exhibited significantly increased tumor sizes compared to vav-cre+ VP16-PPARδ+ mice, yet smaller tumor sizes compared to control (FIGS. 4G and 14F). In addition, tumors grown in vav-cre+ VP16-PPARδ+ Cpt1a^{fl/fl} mice had significantly less CD45+ immune cell and CD8 T cell tumor infiltration compared to vav-cre+ VP16-PPARδ+ mice (FIGS. 4H, 4I, 14G, and 14H). Thus, Cpt1a mediated mitochondrial fatty acid β-oxidation was identified as a metabolic mechanism that partially mediates the effects of PPARδ activation on anti-tumor immunity.

### Additional Materials and Methods

The following materials and methods were used to generate the data shown in the indicated figures.

### FIG. 13M—Cpt1a KO C14 Assay

Fatty acid oxidation to CO₂ and acid-soluble products (ASP) were measured in primary CD8 T cells cultured in 25-cm² flasks. Conjugation of [1-¹⁴C] palmitate to BSA was done by dissolving 1 g of fatty acid free BSA (Proliant Biologicals, 68700) in 5.5 ml 0.9% NaCl by stirring and heating in a water bath at 40° C. Then 6.97 mg of cold palmitate (Sigma-Aldrich, P9767) and 1 ml of 0.1N NaOH were mixed and heated at 90° C. in a heat-block until the solution was clear. The cold palmitate-NaOH solution was added rapidly drop by drop into the BSA solution. Then 2.5 ml of [1-¹⁴C]palmitic acid (PerkinElmer, NEC075H250UC) was added drop by drop into the BSA solution. The final solution was then filtered through a 0.45 um filter and stored at −20° C.
Murine spleens were mechanically digested, strained through 40 um cell strainer and centrifuged at 300 g for 5 minutes. Red blood cells were lysed with ACK lysis buffer (CSHL, homemade) for 3 minutes. Splenic CD8 T cells were isolated using negative magnetic selection according to manufacturer's instructions (Stemcell Technologies, 19853). 1×10⁶ CD8+ T cells/ml were plated in either 96- or 24-well plates pre-coated with 2 μg/ml anti-CD3 and anti-CD8 antibodies (Biolegend, 100340, 102116) and incubated in primary T cell media (RPMI (Corning, 10-040-CV) supplemented with 10% fetal bovine serum (FBS), 1% penicillin-streptomycin (P/S), 50 uM β-mercaptoethanol (MilliporeSigma, M3148-25ML), and 20 ng/ml recombinant mouse IL-2 (R&D systems, 402-ML-020) at 37° C. in a humidified 5% CO₂ incubator for 72 hours.
After 72 hours of activation, cells were washed with Krebs-Ringer bicarbonate buffer containing 1 M HEPES (Thermo Fisher Scientific, 15630080) (KRBH) and 0.1% BSA. Cells were then incubated for 30 minutes at 37° C. in KRBH containing 1% BSA. Cells were washed again with KRBH containing 0.1% BSA, and 2×10⁶ cells were plated per flask. Per condition, one flask was harvested with 500 ul RIPA protein lysis buffer (Thermo Fisher Scientific, 89900) for protein quantification using Pierce BCA protein assay kit (Thermo Fisher Scientific, 23225). Cells were incubated for 3 hours at 37° C. in 2 ml of fresh KRBH in the presence of 0.8 mM L-carnitine (Sigma-Aldrich, C0158), 2.5 or 25 mM glucose (Agilent, 103577-100), and 0.25 mM [1-¹⁴C]palmitate. For oxidation measurements, the 25-cm² flasks were sealed at the beginning of the incubation with a rubber stopper holding a 3-cm length of PVC tubing containing a 1 cm² piece of Whatman paper soaked in 0.1N KOH. At the end of the incubation period, 200 ul of perchloric acid (Sigma-Aldrich, 244252) (40% vol/vol) was injected into each flask via a needle through the rubber stopper to acidify the medium and stop the reaction. Flasks were left overnight at room temperature, and then Whatman papers were removed and added into separate vials containing 5 ml of scintillation liquid (PerkinElmer, 6013321). The perchloric acid-treated medium was centrifuged at 14,000 g for 10 minutes, and 800 ul of the supernatant was added into separate vials containing 5 ml of scintillation liquid. Fatty acid oxidation into ASP was measured by liquid scintillation counting from the supernatants containing the labeled ASPs after being left overnight at room temperature in the scintillation liquid. Bound ¹⁴CO₂ in the Whatman paper was measured by liquid scintillation counting after being left overnight at room temperature in the scintillation liquid. Oxidation results are expressed as: nmol of palmitate.mg⁻¹ prot.h⁻¹=((CPM of sample−Blank flask CPM)×500)/(total CPM×mg protein×h). CPM is counts per minute the liquid scintillation counter detects. Where 500 is the total nmol of palmitate per flask, total CPM are the counts resulting from directly counting on the scintillation liquid 200 ul of 2.5 mM [1-¹⁴C]palmitate used per flask, mg protein is from the BCA assay and h is the time of incubation. ASP results are expressed as: nmol of palmitate.mg⁻¹ prot.h⁻¹=((CPM of sample−Blank flask CPM)×500×(2200/800))/(total CPM×mg protein×h). Where 500 is the total nmol of palmitate per flask, total CPM are the counts resulting from directly counting on the scintillation liquid 200 ul of 2.5 mM [1-¹⁴C]palmitate used per flask, 2200/800 is the dilution factor, mg protein is from the BCA assay and h is the time of incubation. Total palmitate oxidation is the sum of both oxidation and ASP results.

### Example 6: Cell Intrinsic PPARδ Activation is Sufficient to Enhance Human CAR-T Cell Cytotoxicity

CAR-T cells are genetically engineered T cells that express recombinant receptors which are involved in direct antigen binding and T cell activation (Sadelain et al., 2013). Unlike T cells, CAR-T cells do not require peptide presentation on self-MHC molecules, and can directly engage with tumor antigens. Once bound to target antigens, CAR signaling domains lead to robust T cell activation and cytotoxic function. CD19 targeting CAR-Ts have shown unprecedented efficacy for B cell malignancies. However, CAR-T therapies cannot effectively eliminate solid tumors due to many challenges such as immunosuppressive tumor microenvironments, impaired tumor infiltration, and downregulated target antigen on cancer cell surfaces (Sterner and Sterner, 2021).

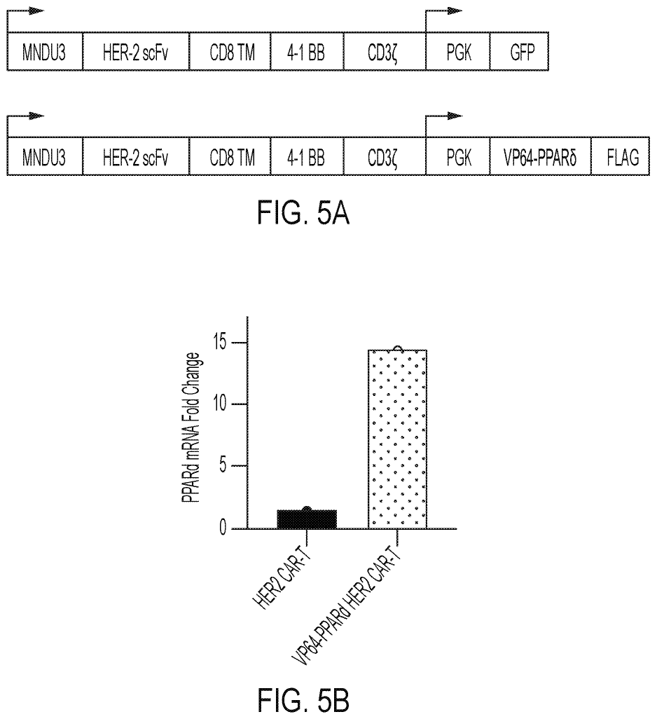

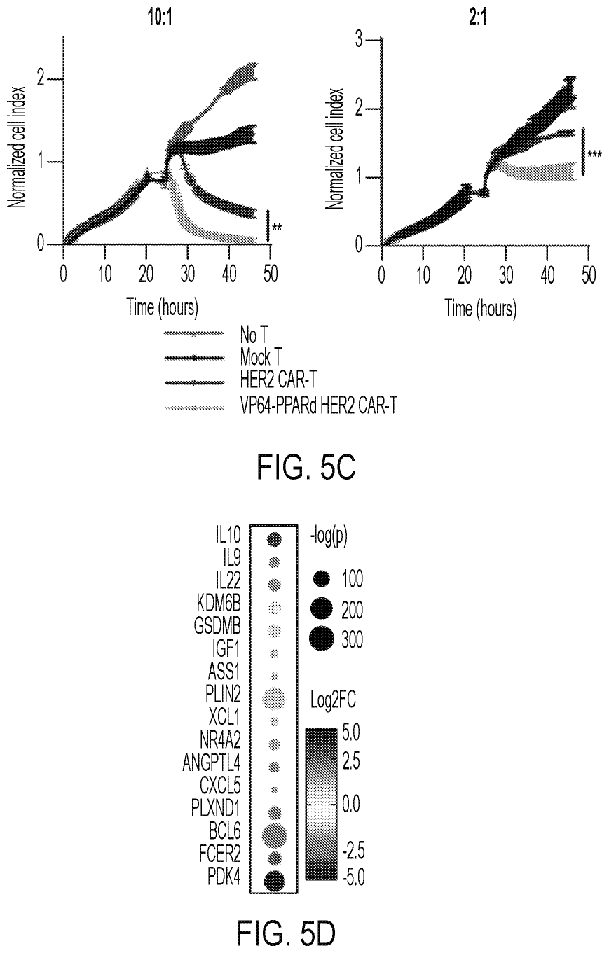

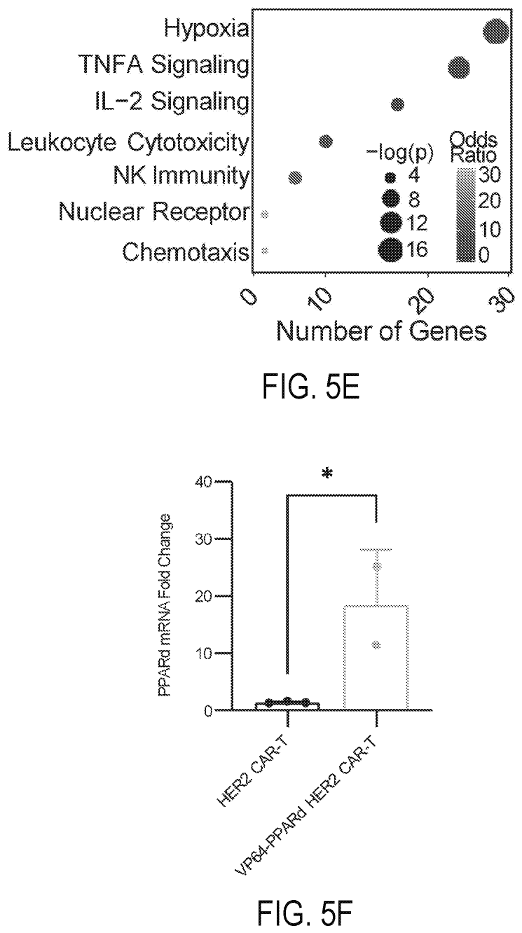

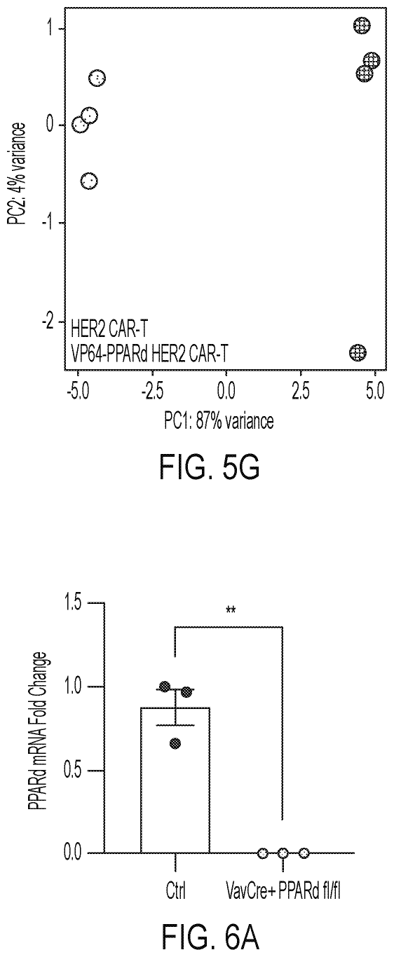

In order to study the effects of genetic PPARδ gain-of-function on CAR-T cell effector function in an additional cancer model that poorly respond to CAR-T therapies, the focus CAR-T cells that target HER-2 receptor overexpressed by several tumor types such as breast and ovarian cancer (see, e.g., Budi et al., 2022). Accordingly, a novel human CAR-T cell was generated that targets human epidermal growth factor receptor 2 (HER-2) and has PPARδ gain-of-function driven by VP64-PPARδ fusion protein (Beerli et al., 1998; Sadowski et al., 1988) (FIG. 5A). PPARδ over-expression was confirmed by q-RT-PCR (FIG. 5B and FIG. 5F). When co-cultured with HER-2 overexpressing ovarian cancer cell line SKOV3, VP64-PPARδ CAR-T cells exhibited significantly higher cytotoxic function compared to WT CAR-Ts at effector:target ratios 10:1 and 2:1. (FIG. 5C). When assessing gene expression changes in these engineered CAR T-cells, reduced expression of anti-inflammatory cytokines such as IL-10 and increased expression of genes related to fatty acid metabolism (PDK4, ANGPTL4, PLIN2), and migration (CXCL5, XCL1) (FIGS. 5D and 5G) was observed. Moreover, pathway enrichment analysis demonstrated upregulation in gene signatures associated increased immune cell function and cytotoxicity (FIG. 5E). Thus, it was determined that pharmacologic or genetic PPARδ activation enhanced human CAR-T cell cytotoxicity in vitro, irrespective of target antigen or cancer type.

### Additional Materials and Methods

The following materials and methods were used to generate the data shown in the indicated figures.

### FIGS. 5D-5E, 5G—HER2 CART RNAseq

For bulk RNA sequencing, CAR-T cells were thawed and cultured for 5 days in human T cell media before RNA was extracted. For this, cells were centrifuged, supernatants were discarded and cell pellets were resuspended in 300 ul TRIzol reagent (Thermo Fisher, 15596026). RNA was isolated using Direct-zol RNA Microprep kit (Zymo, R2062), according to manufacturer's instructions. Bulk RNA sequencing libraries were prepared using NebNext Ultra II kit (New England BioLabs, E7760) and sequenced using Illumina NextSeq. Raw outputs were trimmed with trim galore (v0.6.7) and aligned to GRCh37 using STAR (v2.7.2b, (Dobin et al., Bioinformatics. 2013 January; 29(1):15-21)). Aligned counts were quantified using Salmon (v1.5.2, (Patro et al., Nature Methods 2017 14:4. 2017 Mar. 6; 14(4):417-9)). StringTie was used for transcript assembly (v2.2.1, (Pertea et al., Nature Biotechnology 2015 33:3. 2015 Feb. 18; 33(3):290-5)). Read and alignment quality were analyzed with RSeqQC (v3.0.1, (Wang et al., Bioinformatics. 2012 August; 28(16):2184)) and summarized with MultiQC (v1.9, (Ewels et al., Bioinformatics. 2016 Oct. 1; 32(19):3047-8)). Differential gene expression between control and experimental samples was assessed with DEseq2 (v1.28, (Love et al., Genome Biol. 2014 Dec. 5; 15(12):1-21)), fitting a model with fixed effects for sequencing batch effect and treatment. Contrast for treatment were extracted and transcripts considered differentially expressed with an absolute fold change greater than log 2 (Westcott et al., Nature Cancer 2021 2:10. 2021 Sep. 30; 2(10):1071-85) and adjusted p-value of less than 0.05. For visualization purposes, heatmaps plots were generated with ggplot2 (v2.6.2, (Wickham, Ggplot2: elegant graphics for data analysis. p212, Use R! (Springer, New York, 2009))), volcano plots were generated with EnhancedVolcano (v1.8.0). Gene set enrichment analysis was conducted using EnrichR (v3.2, (Kuleshov et al., Nucleic Acids Res. 2016 Jul. 8; 44(W1):W90-7)). Analysis code is available on GitHub (github.com/Vyoming/A-metabolic-switch-that-boosts-immune-cell-fitness-against-cancer.git).

### FIG. 5F—PPARD Upregulation in HER2 CART

CAR-T experiments were performed in collaboration with ProMab Biotechnologies, Inc. GFP reporter in CAR-T cells targeting human HER-2 protein (Promab, PM-CAR1064, MNDU3-HER2 scFv-CD8 TM-41BB-CD3z-PGK-GFP) was replaced with VP64-human PPAR delta-FLAG sequence in order to promote over-expression and over-activation of human PPAR delta in HER-2 CAR-T cells. Demonstration of PPARD upregulation was performed using qRT-PCR.

### Example 7: PPARδ Activation in Regulatory T Cells (T_{regs}) Promotes Anti-Tumor Immunity

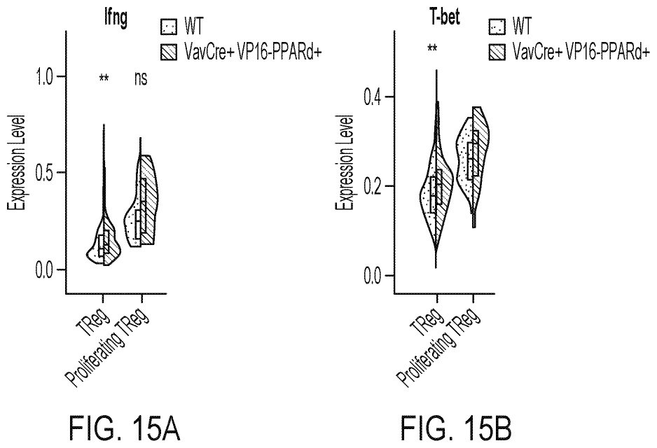

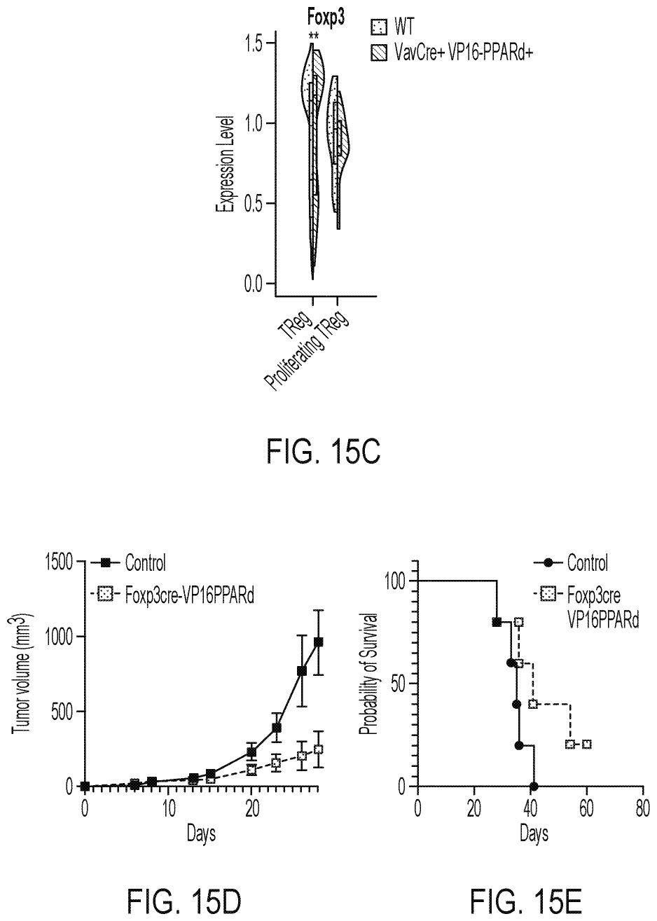

It was determined that tumor infiltrating vav-cre+ VP16-PPARδ+ T_{regs} and proliferating T_{regs} exhibited hallmarks of T_{reg} destabilization such as foxp3 downregulation and upregulation of genes associated with effector T cell function such as ifng and tbx21(t-bet) (Overacre and Vignali, 2016) (FIGS. 15A-15C). Building on these observations, it was assessed if T_{reg} specific PPARδ activation could be sufficient to promote anti-tumor immunity. In order to test this, Foxp3cre+ VP16-PPARδ+ mice were generated that over-expressed PPARδ gain-of-function fusion protein VP16-PPARδ in T_{regs}. To determine the effects of PPARδ activation in T_{regs} on anti-tumor immune function, Foxp3cre+ VP16-PPARδ+ mice were inoculated with subcutaneous MC38 tumors and followed tumor growth. Remarkably, it was determined that PPARδ activation in T_{regs} was sufficient to promote anti-tumor immunity and enhance survival (FIGS. 15D, 15E). Thus, it was determined that PPARδ activation in T_{regs} led to T_{reg} destabilization and enhance anti-tumor immunity.

### Example 8: Cell-Intrinsic PPARδ Activation Synergizes with Immunotherapy

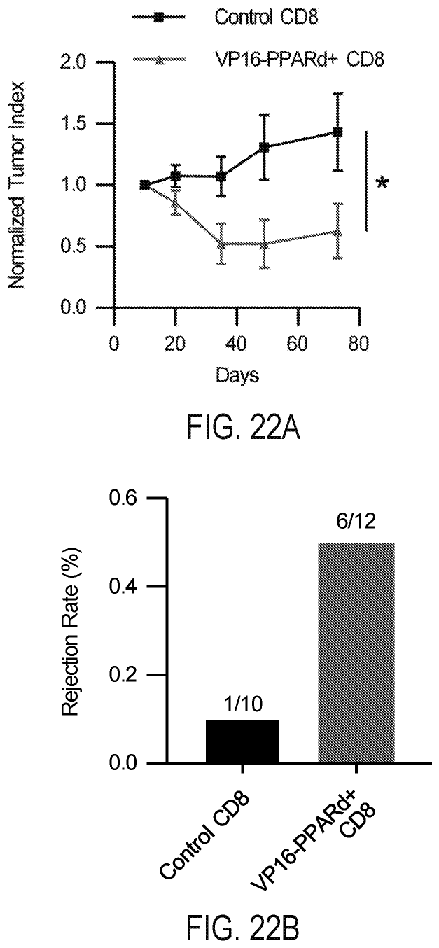

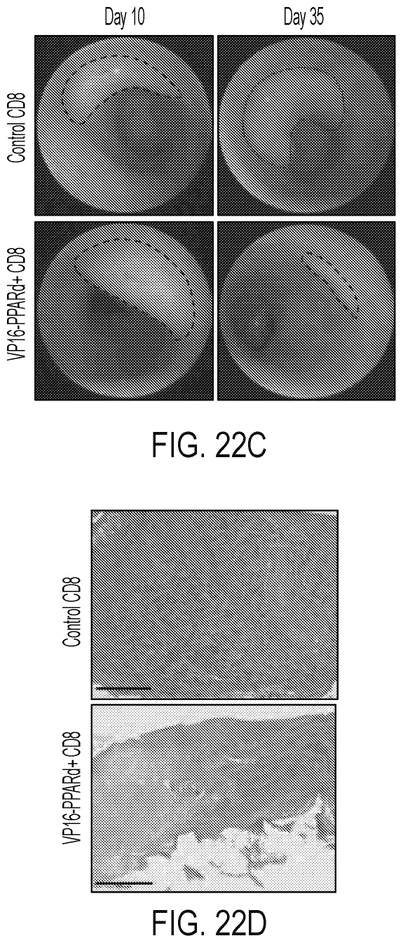

In order to explore the therapeutic applications of the findings presented herein, the synergistic effects of targeting PPARδ together with anti-PD-1 checkpoint blockade were assessed. Transferring in vitro stimulated VP16-PPARδ+ CD8 T cells into mice harboring established AKPS tumors lead to significantly reduced tumor growth compared to CD8 T cells transferred from wild-type mice when concomitantly treated with anti-PD-1 antibodies (FIGS. 22A-22C). Strikingly, 50% of mice that received VP16-PPARδ+ CD8 T cells rejected their tumors by day 35 (FIG. 22B). Histological examination of tumors that remained in mice treated with CD8 T-cells harboring active PPARδ by a pathologist showed large areas of fibrosis, with most of tumor eliminated by the immune system (FIG. 22D). Thus, activating PPARδ signaling can act in synergy with other modes of immunotherapy including immune checkpoint blockade.

### Additional Materials and Methods

The following materials and methods were used to generate the data shown in the indicated figures.

### FIGS. 22A-22C—Adoptive T-Cell Transfer

1×10⁶ AKPS cells were injected into colon sub-mucosa of C57BL/6J mice as described above. 7 days post-implantation tumors were confirmed via colonoscopy and on day 9 mice were sub-lethally irradiated using 5 Gy total body irradiation. CD8 T cells were isolated from control or vav-cre+ VP16-PPARδ+ mice and stimulated for 72 hours as described above. 48 hours after mice were irradiated, 3×10⁶ CD8 T cells were intravenously injected. Mice were treated with anti-PD-1 antibodies (200 ug, BioXCell, clone RMP1-14) intraperitoneally every 2-3 days for 14 days. Tumor growth was assessed using colonoscopy and mice were euthanized at humane endpoint. Tumor index was calculated by dividing the tumor diameter by the colon diameter using colonoscopy images. Each tumor index was normalized to their respective week-1 tumor index. Representative colonoscopy images were acquired at day 10 and day 35 of mice that received wild-type CD8 T cells, or VP16-PPARδ CD8 T cells with anti-PD-1 after AKPS tumor inoculation.

### FIG. 22D—Adoptive T-Cell Transfer H&E

Tumor tissues from mice in FIGS. 22A-22C were fixed and processed as described above. FFPE tumor slides were stained with hematoxylin and eosin.

### REFERENCES

Beerli, R. R., Segal, D. J., Dreier, B., and Barbas, C. F., 3rd (1998). Toward controlling gene expression at will: specific regulation of the erbB-2/HER-2 promoter by using polydactyl zinc finger proteins constructed from modular building blocks. Proc Natl Acad Sci USA 95, 14628-14633.
Berger, J., and Moller, D. E. (2002). The mechanisms of action of PPARs. Annu Rev Med 53, 409-435.
Beyaz, S., Mana, M. D., Roper, J., Kedrin, D., Saadatpour, A., Hong, S. J., Bauer-Rowe, K. E., Xifaras, M. E., Akkad, A., Arias, E., et al. (2016). High-fat diet enhances sternness and tumorigenicity of intestinal progenitors. Nature 531, 53-58.
Beyaz, S., Mana, M. D., and Yilmaz, O. H. (2021). High-fat diet activates a PPAR-delta program to enhance intestinal stem cell function. Cell Stem Cell 28, 598-599.
Beyaz, S., Roper, J., Bauer-Rowe, K. E., Xifaras, M. E., Ergin, I., Dohnalova, L., Biton, M., Shekar, K., Mou, H., Eskiocak, O., et al. (2020). Dietary suppression of MHC-II expression in intestinal stem cells enhances intestinal tumorigenesis. bioRxiv, 2020.2009.2005.284174.
Bidwell, B. N., Slaney, C. Y., Withana, N. P., Forster, S., Cao, Y., Loi, S., Andrews, D., Mikeska, T., Mangan, N. E., Samarajiwa, S. A., et al. (2012). Silencing of Irf7 pathways in breast cancer cells promotes bone metastasis through immune escape. Nat Med 18, 1224-1231.
Browaeys, R., Saelens, W., and Saeys, Y. (2020). NicheNet: modeling intercellular communication by linking ligands to target genes. Nat Methods 17, 159-162.
Budi, H. S., Ahmad, F. N., Achmad, H., Ansari, M. J., Mikhailova, M. V., Suksatan, W., Chupradit, S., Shomali, N., and Marofi, F. (2022). Human epidermal growth factor receptor 2 (HER2)-specific chimeric antigen receptor (CAR) for tumor immunotherapy; recent progress. Stem Cell Res Ther 13, 40.
Butler, L. M., Perone, Y., Dehairs, J., Lupien, L. E., de Laat, V., Talebi, A., Loda, M., Kinlaw, W. B., and Swinnen, J. V. (2020). Lipids and cancer: Emerging roles in pathogenesis, diagnosis and therapeutic intervention. Adv Drug Deliv Rev 159, 245-293.
Chabowski, A., Gorski, J., Luiken, J. J., Glatz, J. F., and Bonen, A. (2007). Evidence for concerted action of FAT/CD36 and FABPpm to increase fatty acid transport across the plasma membrane. Prostaglandins Leukot Essent Fatty Acids 77, 345-353.
Chamoto, K., Chowdhury, P. S., Kumar, A., Sonomura, K., Matsuda, F., Fagarasan, S., and Honjo, T. (2017). Mitochondrial activation chemicals synergize with surface receptor PD-1 blockade for T cell-dependent antitumor activity. Proc Natl Acad Sci USA 114, E761-E770.
Chang, C. H., and Pearce, E. L. (2016). Emerging concepts of T cell metabolism as a target of immunotherapy. Nat Immunol 17, 364-368.
Chang, C. H., Qiu, J., O'Sullivan, D., Buck, M. D., Noguchi, T., Curtis, J. D., Chen, Q., Gindin, M., Gubin, M. M., van der Windt, G. J., et al. (2015). Metabolic Competition in the Tumor Microenvironment Is a Driver of Cancer Progression. Cell 162, 1229-1241.
Charo, J., Finkelstein, S. E., Grewal, N., Restifo, N. P., Robbins, P. F., and Rosenberg, S. A. (2005). Bcl-2 overexpression enhances tumor-specific T-cell survival. Cancer Res 65, 2001-2008.
Chow, M. T., Ozga, A. J., Servis, R. L., Frederick, D. T., Lo, J. A., Fisher, D. E., Freeman, G. J., Boland, G. M., and Luster, A. D. (2019). Intratumoral Activity of the CXCR3 Chemokine System Is Required for the Efficacy of Anti-PD-1 Therapy. Immunity 50, 1498-1512 e1495.
Chowdhury, P. S., Chamoto, K., Kumar, A., and Honjo, T. (2018). PPAR-Induced Fatty Acid Oxidation in T Cells Increases the Number of Tumor-Reactive CD8(+) T Cells and Facilitates Anti-PD-1 Therapy. Cancer Immunol Res 6, 1375-1387.
Christofides, A., Konstantinidou, E., Jani, C., and Boussiotis, V. A. (2021). The role of peroxisome proliferator-activated receptors (PPAR) in immune responses. Metabolism 114, 154338.
Clausen, B. E., Burkhardt, C., Reith, W., Renkawitz, R., and Forster, I. (1999). Conditional gene targeting in macrophages and granulocytes using LysMcre mice. Transgenic Res 8, 265-277.
Conrady, C. D., Zheng, M., Mandal, N. A., van Rooijen, N., and Carr, D. J. (2013). IFN-alpha-driven CCL2 production recruits inflammatory monocytes to infection site in mice. Mucosal Immunol 6, 45-55.
Corbett, T. H., Griswold, D. P., Jr., Roberts, B. J., Peckham, J. C., and Schabel, F. M., Jr. (1975). Tumor induction relationships in development of transplantable cancers of the colon in mice for chemotherapy assays, with a note on carcinogen structure. Cancer Res 35, 2434-2439.
Curmi, P. A., Nogues, C., Lachkar, S., Carelle, N., Gonthier, M. P., Sobel, A., Lidereau, R., and Bieche, I. (2000). Overexpression of stathmin in breast carcinomas points out to highly proliferative tumours. Br J Cancer 82, 142-150.
DePeaux, K., and Delgoffe, G. M. (2021). Metabolic barriers to cancer immunotherapy. Nat Rev Immunol 21, 785-797.
Efremova, M., Rieder, D., Klepsch, V., Charoentong, P., Finotello, F., Hackl, H., Hermann-Kleiter, N., Lower, M., Baier, G., Krogsdam, A., et al. (2018). Targeting immune checkpoints potentiates immunoediting and changes the dynamics of tumor evolution. Nat Commun 9, 32.
Embgenbroich, M., and Burgdorf, S. (2018). Current Concepts of Antigen Cross-Presentation. Front Immunol 9, 1643.
Ewens, A., Mihich, E., and Ehrke, M. J. (2005). Distant metastasis from subcutaneously grown E0771 medullary breast adenocarcinoma. Anticancer Res 25, 3905-3915.
Fensterl, V., and Sen, G. C. (2015). Interferon-induced Ifit proteins: their role in viral pathogenesis. J Virol 89, 2462-2468.
Fidler, I. J. (1975). Biological behavior of malignant melanoma cells correlated to their survival in vivo. Cancer Res 35, 218-224.
Fridman, W. H., Pages, F., Sautes-Fridman, C., and Galon, J. (2012). The immune contexture in human tumours: impact on clinical outcome. Nat Rev Cancer 12, 298-306.
Georgiades, P., Ogilvy, S., Duval, H., Licence, D. R., Charnock-Jones, D. S., Smith, S. K., and Print, C. G. (2002). VavCre transgenic mice: a tool for mutagenesis in hematopoietic and endothelial lineages. Genesis 34, 251-256.
Getachew, Y., Stout-Delgado, H., Miller, B. C., and Thiele, D. L. (2008). Granzyme C supports efficient CTL-mediated killing late in primary alloimmune responses. J Immunol 181, 7810-7817.
Gokerkucuk, E. B., Tramier, M., and Bertolin, G. (2020). Imaging Mitochondrial Functions: from Fluorescent Dyes to Genetically-Encoded Sensors. Genes (Basel) 11.
Golstein, P., and Griffiths, G. M. (2018). An early history of T cell-mediated cytotoxicity. Nat Rev Immunol 18, 527-535.
Grasso, C. S., Giannakis, M., Wells, D. K., Hamada, T., Mu, X. J., Quist, M., Nowak, J. A., Nishihara, R., Qian, Z. R., Inamura, K., et al. (2018). Genetic Mechanisms of Immune Evasion in Colorectal Cancer. Cancer Discov 8, 730-749.
Groom, J. R., and Luster, A. D. (2011a). CXCR3 in T cell function. Exp Cell Res 317, 620-631.
Groom, J. R., and Luster, A. D. (2011b). CXCR3 ligands: redundant, collaborative and antagonistic functions. Immunol Cell Biol 89, 207-215.
Gurjao, C., Tsukrov, D., Imakaev, M., Luquette, L. J., and Mirny, L. A. (2020). Limited evidence of tumour mutational burden as a biomarker of response to immunotherapy. bioRxiv, 2020.2009.2003.260265.
Hamarsheh, S., Gross, O., Brummer, T., and Zeiser, R. (2020). Immune modulatory effects of oncogenic KRAS in cancer. Nat Commun 11, 5439.
Haslam, A., and Prasad, V. (2019). Estimation of the Percentage of US Patients With Cancer Who Are Eligible for and Respond to Checkpoint Inhibitor Immunotherapy Drugs. JAMA Netw Open 2, e192535.
Hellstrom, I., Hellstrom, K. E., Pierce, G. E., and Yang, J. P. (1968). Cellular and humoral immunity to different types of human neoplasms. Nature 220, 1352-1354.
Honda, K., Takaoka, A., and Taniguchi, T. (2006). Type I interferon gene induction by the interferon regulatory factor family of transcription factors. Immunity 25, 349-360.
Honda, K., Yanai, H., Negishi, H., Asagiri, M., Sato, M., Mizutani, T., Shimada, N., Ohba, Y., Takaoka, A., Yoshida, N., et al. (2005). IRF-7 is the master regulator of type-I interferon-dependent immune responses. Nature 434, 772-777.
Iwasaki, A., and Medzhitov, R. (2015). Control of adaptive immunity by the innate immune system. Nat Immunol 16, 343-353.
Kanehisa, M., and Goto, S. (2000). KEGG: kyoto encyclopedia of genes and genomes. Nucleic Acids Res 28, 27-30.
Kennedy, L. B., and Salama, A. K. S. (2020). A review of cancer immunotherapy toxicity. CA Cancer J Clin 70, 86-104.
Kim, T., Zhelyabovska, O., Liu, J., and Yang, Q. (2013). Generation of an inducible, cardiomyocyte-specific transgenic mouse model with PPAR beta/delta overexpression. Methods Mol Biol 952, 57-65.
Kondo, T., Takata, H., and Takiguchi, M. (2007). Functional expression of chemokine receptor CCR6 on human effector memory CD8+ T cells. Eur J Immunol 37, 54-65.
Korant, B. D., Blomstrom, D. C., Jonak, G. J., and Knight, E., Jr. (1984). Interferon-induced proteins. Purification and characterization of a 15,000-dalton protein from human and bovine cells induced by interferon. J Biol Chem 259, 14835-14839.
Kronke, G., Kadl, A., Ikonomu, E., Bluml, S., Furnkranz, A., Sarembock, I. J., Bochkov, V. N., Exner, M., Binder, B. R., and Leitinger, N. (2007). Expression of heme oxygenase-1 in human vascular cells is regulated by peroxisome proliferator-activated receptors. Arterioscler Thromb Vasc Biol 27, 1276-1282.
LaFleur, M. W., Nguyen, T. H., Coxe, M. A., Miller, B. C., Yates, K. B., Gillis, J. E., Sen, D. R., Gaudiano, E. F., Al Abosy, R., Freeman, G. J., et al. (2019). PTPN2 regulates the generation of exhausted CD8(+) T cell subpopulations and restrains tumor immunity. Nat Immunol 20, 1335-1347.
Landolfo, S., Gariglio, M., Gribaudo, G., and Lembo, D. (1998). The Ifi 200 genes: an emerging family of IFN-inducible genes. Biochimie 80, 721-728.
Lane, A. N., and Fan, T. W. (2015). Regulation of mammalian nucleotide metabolism and biosynthesis. Nucleic Acids Res 43, 2466-2485.
Le, D. T., Durham, J. N., Smith, K. N., Wang, H., Bartlett, B. R., Aulakh, L. K., Lu, S., Kemberling, H., Wilt, C., Luber, B. S., et al. (2017). Mismatch repair deficiency predicts response of solid tumors to PD-1 blockade. Science 357, 409-413.
Li, H., Wu, M., and Zhao, X. (2022). Role of chemokine systems in cancer and inflammatory diseases. MedComm (2020) 3, e147.
Liang, H., and Ward, W. F. (2006). PGC-lalpha: a key regulator of energy metabolism. Adv Physiol Educ 30, 145-151.
Liberzon, A., Birger, C., Thorvaldsdottir, H., Ghandi, M., Mesirov, J. P., and Tamayo, P. (2015). The Molecular Signatures Database (MSigDB) hallmark gene set collection. Cell Syst 1, 417-425.
Lin, J., Handschin, C., and Spiegelman, B. M. (2005). Metabolic control through the PGC-1 family of transcription coactivators. Cell Metab 1, 361-370.
Liu, Y., Colby, J. K., Zuo, X., Jaoude, J., Wei, D., and Shureiqi, I. (2018). The Role of PPAR-delta in Metabolism, Inflammation, and Cancer: Many Characters of a Critical Transcription Factor. Int J Mol Sci 19.
Lochner, M., Berod, L., and Sparwasser, T. (2015). Fatty acid metabolism in the regulation of T cell function. Trends Immunol 36, 81-91.
Luke, J. J., Bao, R., Sweis, R. F., Spranger, S., and Gajewski, T. F. (2019). WNT/beta-catenin Pathway Activation Correlates with Immune Exclusion across Human Cancers. Clin Cancer Res 25, 3074-3083.
Mantovani, A., and Sica, A. (2010). Macrophages, innate immunity and cancer: balance, tolerance, and diversity. Curr Opin Immunol 22, 231-237.
Manzo, T., Prentice, B. M., Anderson, K. G., Raman, A., Schalck, A., Codreanu, G. S., Nava Lauson, C. B., Tiberti, S., Raimondi, A., Jones, M. A., et al. (2020). Accumulation of long-chain fatty acids in the tumor microenvironment drives dysfunction in intrapancreatic CD8+ T cells. J Exp Med 217.
Michelet, X., Dyck, L., Hogan, A., Loftus, R. M., Duquette, D., Wei, K., Beyaz, S., Tavakkoli, A., Foley, C., Donnelly, R., et al. (2018). Metabolic reprogramming of natural killer cells in obesity limits antitumor responses. Nat Immunol 19, 1330-1340.
Munn, D. H., Sharma, M. D., and Johnson, T. S. (2018). Treg Destabilization and Reprogramming: Implications for Cancer Immunotherapy. Cancer Res 78, 5191-5199.
Neels, J. G., and Grimaldi, P. A. (2014). Physiological functions of peroxisome proliferator-activated receptor beta. Physiol Rev 94, 795-858.
Nelson, D. M., Ye, X., Hall, C., Santos, H., Ma, T., Kao, G. D., Yen, T. J., Harper, J. W., and Adams, P. D. (2002). Coupling of DNA synthesis and histone synthesis in S phase independent of cyclin/cdk2 activity. Mol Cell Biol 22, 7459-7472.
Ning, S., Pagano, J. S., and Barber, G. N. (2011). IRF7: activation, regulation, modification and function. Genes Immun 12, 399-414.
Overacre, A. E., and Vignali, D. A. (2016). T(reg) stability: to be or not to be. Curr Opin Immunol 39, 39-43.
Pan, Z. K., Weiskirch, L. M., and Paterson, Y. (1999). Regression of established B16F10 melanoma with a recombinant Listeria monocytogenes vaccine. Cancer Res 59, 5264-5269.
Parker, B. S., Rautela, J., and Hertzog, P. J. (2016). Antitumour actions of interferons: implications for cancer therapy. Nat Rev Cancer 16, 131-144.
Pelka, K., Hofree, M., Chen, J. H., Sarkizova, S., Pirl, J. D., Jorgji, V., Bejnood, A., Dionne, D., Ge, W. H., Xu, K. H., et al. (2021). Spatially organized multicellular immune hubs in human colorectal cancer. Cell 184, 4734-4752 e4720.
Philip, M., and Schietinger, A. (2022). CD8(+) T cell differentiation and dysfunction in cancer. Nat Rev Immunol 22, 209-223.
Picard, E., Verschoor, C. P., Ma, G. W., and Pawelec, G. (2020). Relationships Between Immune Landscapes, Genetic Subtypes and Responses to Immunotherapy in Colorectal Cancer. Front Immunol 11, 369.
Pidugu, V. K., Pidugu, H. B., Wu, M. M., Liu, C. J., and Lee, T. C. (2019). Emerging Functions of Human IFIT Proteins in Cancer. Front Mol Biosci 6, 148.
Raskov, H., Orhan, A., Christensen, J. P., and Gogenur, I. (2021). Cytotoxic CD8(+) T cells in cancer and cancer immunotherapy. Br J Cancer 124, 359-367.
Raud, B., Roy, D. G., Divakaruni, A. S., Tarasenko, T. N., Franke, R., Ma, E. H., Samborska, B., Hsieh, W. Y., Wong, A. H., Stuve, P., et al. (2018). Etomoxir Actions on Regulatory and Memory T Cells Are Independent of Cpt1a-Mediated Fatty Acid Oxidation. Cell Metab 28, 504-515 e507.
Reinfeld, B. I., Madden, M. Z., Wolf, M. M., Chytil, A., Bader, J. E., Patterson, A. R., Sugiura, A., Cohen, A. S., Ali, A., Do, B. T., et al. (2021). Cell-programmed nutrient partitioning in the tumour microenvironment. Nature 593, 282-288.
Ringel, A. E., Drijvers, J. M., Baker, G. J., Catozzi, A., Garcia-Canaveras, J. C., Gassaway, B. M., Miller, B. C., Juneja, V. R., Nguyen, T. H., Joshi, S., et al. (2020). Obesity Shapes Metabolism in the Tumor Microenvironment to Suppress Anti-Tumor Immunity. Cell 183, 1848-1866 e1826.
Roper, J., Tammela, T., Akkad, A., Almeqdadi, M., Santos, S. B., Jacks, T., and Yilmaz, O. H. (2018). Colonoscopy-based colorectal cancer modeling in mice with CRISPR-Cas9 genome editing and organoid transplantation. Nat Protoc 13, 217-234.
Roper, J., Tammela, T., Cetinbas, N. M., Akkad, A., Roghanian, A., Rickelt, S., Almeqdadi, M., Wu, K., Oberli, M. A., Sanchez-Rivera, F. J., et al. (2017). In vivo genome editing and organoid transplantation models of colorectal cancer and metastasis. Nat Biotechnol 35, 569-576.
Sadelain, M., Brentjens, R., and Riviere, I. (2013). The basic principles of chimeric antigen receptor design. Cancer Discov 3, 388-398.
Sadowski, I., Ma, J., Triezenberg, S., and Ptashne, M. (1988). GAL4-VP16 is an unusually potent transcriptional activator. Nature 335, 563-564.
Sahai, E., Astsaturov, I., Cukierman, E., DeNardo, D. G., Egeblad, M., Evans, R. M., Fearon, D., Greten, F. R., Hingorani, S. R., Hunter, T., et al. (2020). A framework for advancing our understanding of cancer-associated fibroblasts. Nat Rev Cancer 20, 174-186.
Sahin, I. H., Ciombor, K. K., Diaz, L. A., Yu, J., and Kim, R. (2022). Immunotherapy for Microsatellite Stable Colorectal Cancers: Challenges and Novel Therapeutic Avenues. Am Soc Clin Oncol Educ Book 42, 1-12.
Saibil, S. D., St Paul, M., Laister, R. C., Garcia-Batres, C. R., Israni-Winger, K., Elford, A. R., Grimshaw, N., Robert-Tissot, C., Roy, D. G., Jones, R. G., et al. (2019). Activation of Peroxisome Proliferator-Activated Receptors alpha and delta Synergizes with Inflammatory Signals to Enhance Adoptive Cell Therapy. Cancer Res 79, 445-451.
Schietinger, A., Philip, M., Krisnawan, V. E., Chiu, E. Y., Delrow, J. J., Basom, R. S., Lauer, P., Brockstedt, D. G., Knoblaugh, S. E., Hammerling, G. J., et al. (2016). Tumor-Specific T Cell Dysfunction Is a Dynamic Antigen-Driven Differentiation Program Initiated Early during Tumorigenesis. Immunity 45, 389-401.
Schlaepfer, I. R., and Joshi, M. (2020). CPT1A-mediated Fat Oxidation, Mechanisms, and Therapeutic Potential. Endocrinology 161.
Schoors, S., Bruning, U., Missiaen, R., Queiroz, K. C., Borgers, G., Elia, I., Zecchin, A., Cantelmo, A. R., Christen, S., Goveia, J., et al. (2015). Fatty acid carbon is essential for dNTP synthesis in endothelial cells. Nature 520, 192-197.
Schouppe, E., De Baetselier, P., Van Ginderachter, J. A., and Sarukhan, A. (2012). Instruction of myeloid cells by the tumor microenvironment: Open questions on the dynamics and plasticity of different tumor-associated myeloid cell populations. Oncoimmunology 1, 1135-1145.
Scott, A. C., Dundar, F., Zumbo, P., Chandran, S. S., Klebanoff, C. A., Shakiba, M., Trivedi, P., Menocal, L., Appleby, H., Camara, S., et al. (2019). TOX is a critical regulator of tumour-specific T cell differentiation. Nature 571, 270-274.
Sharma, P., Hu-Lieskovan, S., Wargo, J. A., and Ribas, A. (2017). Primary, Adaptive, and Acquired Resistance to Cancer Immunotherapy. Cell 168, 707-723.
Shaulian, E., and Karin, M. (2001). AP-1 in cell proliferation and survival. Oncogene 20, 2390-2400.
Shi, L., Wu, L., Wang, S., and Fan, Z. (2009). Granzyme F induces a novel death pathway characterized by Bid-independent cytochrome c release without caspase activation. Cell Death Differ 16, 1694-1706.
Snaebjornsson, M. T., Janaki-Raman, S., and Schulze, A. (2020). Greasing the Wheels of the Cancer Machine: The Role of Lipid Metabolism in Cancer. Cell Metab 31, 62-76.
Sokol, C. L., and Luster, A. D. (2015). The chemokine system in innate immunity. Cold Spring Harb Perspect Biol 7.
Speiser, D. E., Ho, P. C., and Verdeil, G. (2016). Regulatory circuits of T cell function in cancer. Nat Rev Immunol 16, 599-611.
Stadtfeld, M., and Graf, T. (2005). Assessing the role of hematopoietic plasticity for endothelial and hepatocyte development by non-invasive lineage tracing. Development 132, 203-213.
Steimle, V., Siegrist, C. A., Mottet, A., Lisowska-Grospierre, B., and Mach, B. (1994). Regulation of MHC class II expression by interferon-gamma mediated by the transactivator gene CITTA. Science 265, 106-109.
Sterner, R. C., and Sterner, R. M. (2021). CAR-T cell therapy: current limitations and potential strategies. Blood Cancer J 11, 69.
Su, P., Wang, Q., Bi, E., Ma, X., Liu, L., Yang, M., Qian, J., and Yi, Q. (2020). Enhanced Lipid Accumulation and Metabolism Are Required for the Differentiation and Activation of Tumor-Associated Macrophages. Cancer Res 80, 1438-1450.
Sugiura, K., and Stock, C. C. (1952). Studies in a tumor spectrum. I. Comparison of the action of methylbis (2-chloroethyl)amine and 3-bis(2-chloroethyl)aminomethyl-4-methoxymethyl-5-hydroxy-6-methylpyridine on the growth of a variety of mouse and rat tumors. Cancer 5, 382-402.
Sun, X., and Kaufman, P. D. (2018). Ki-67: more than a proliferation marker. Chromosoma 127, 175-186.
Tachibana, K., Yamasaki, D., Ishimoto, K., and Doi, T. (2008). The Role of PPARs in Cancer. PPAR Res 2008, 102737.
Takata, H., Tomiyama, H., Fujiwara, M., Kobayashi, N., and Takiguchi, M. (2004). Cutting edge: expression of chemokine receptor CXCR1 on human effector CD8+ T cells. J Immunol 173, 2231-2235.
Thorne, N., Inglese, J., and Auld, D. S. (2010). Illuminating insights into firefly luciferase and other bioluminescent reporters used in chemical biology. Chem Biol 17, 646-657.
Tirosh, I., Izar, B., Prakadan, S. M., Wadsworth, M. H., 2nd, Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-196.
Vermeulen, K., Van Bockstaele, D. R., and Berneman, Z. N. (2003). The cell cycle: a review of regulation, deregulation and therapeutic targets in cancer. Cell Prolif 36, 131-149.
Vignali, D. A., Collison, L. W., and Workman, C. J. (2008). How regulatory T cells work. Nat Rev Immunol 8, 523-532.
Voss, K., Hong, H. S., Bader, J. E., Sugiura, A., Lyssiotis, C. A., and Rathmell, J. C. (2021). A guide to interrogating immunometabolism. Nat Rev Immunol 21, 637-652.
Wang, H., Franco, F., Tsui, Y. C., Xie, X., Trefny, M. P., Zappasodi, R., Mohmood, S. R., Fernandez-Garcia, J., Tsai, C. H., Schulze, I., et al. (2020). CD36-mediated metabolic adaptation supports regulatory T cell survival and function in tumors. Nat Immunol 21, 298-308.
Wang, Y. X., Zhang, C. L., Yu, R. T., Cho, H. K., Nelson, M. C., Bayuga-Ocampo, C. R., Ham, J., Kang, H., and Evans, R. M. (2004). Regulation of muscle fiber type and running endurance by PPARdelta. PLoS Biol 2, e294.
Westcott, P. M. K., Muyas, F., Smith, O., Hauck, H., Sacks, N. J., Ely, Z. A., Jaeger, A. M., Rideout, W. M., Bhutkar, A., Zhang, D., et al. (2021). Mismatch repair deficiency is not sufficient to increase tumor immunogenicity. bioRxiv, 2021.2008.2024.457572.
Xu, S., Chaudhary, O., Rodriguez-Morales, P., Sun, X., Chen, D., Zappasodi, R., Xu, Z., Pinto, A. F. M., Williams, A., Schulze, I., et al. (2021). Uptake of oxidized lipids by the scavenger receptor CD36 promotes lipid peroxidation and dysfunction in CD8(+) T cells in tumors. Immunity 54, 1561-1577 e1567.
Zhang, Y., Kurupati, R., Liu, L., Zhou, X. Y., Zhang, G., Hudaihed, A., Filisio, F., Giles-Davis, W., Xu, X., Karakousis, G. C., et al. (2017). Enhancing CD8(+) T Cell Fatty Acid Catabolism within a Metabolically Challenging Tumor Microenvironment Increases the Efficacy of Melanoma Immunotherapy. Cancer Cell 32, 377-391 e379.
Zhou, F. (2009). Molecular mechanisms of IFN-gamma to up-regulate MHC class I antigen processing and presentation. Int Rev Immunol 28, 239-260.
Zitvogel, L., Galluzzi, L., Kepp, O., Smyth, M. J., and Kroemer, G. (2015). Type I interferons in anticancer immunity. Nat Rev Immunol 15, 405-414.
   Sequences Associated with the Disclosure

Amino acid sequence encoding a human PPARδ sequence (UniProtKB Accession No. Q03181-1):
MEQPQEEAPEVREEEEKEEVAEAEGAPELNGGPQHALPSSSYTDLSRSSSPPSLLDQL
QMGCDGASCGSLNMECRVCGDKASGFHYGVHACEGCKGFFRRTIRMKLEYEKCER
SCKIQKKNRNKCQYCRFQKCLALGMSHNAIRFGRMPEAEKRKLVAGLTANEGSQYN
PQVADLKAFSKHIYNAYLKNFNMTKKKARSILTGKASHTAPFVIHDIETLWQAEKGL
VWKQLVNGLPPYKEISVHVFYRCQCTTVETVRELTEFAKSIPSFSSLFLNDQVTLLKY
GVHEAIFAMLASIVNKDGLLVANGSGFVTREFLRSLRKPFSDIIEPKFEFAVKFNALEL
DDSDLALFIAAIILCGDRPGLMNVPRVEAIQDTILRALEFHLQANHPDAQYLFPKLLQ
KMADLRQLVTEHAQMMQRIKKTETETSLHPLLQEIYKDMY (SEQ ID NO: 1)
Amino acid sequence encoding a PPARδ sequence:
EQPQEEAPEVREEEEKEEVAEAEGAPELNGGPQHALPSSSYTDLSRSSSPPSLLDQLQ
MGCDGASCGSLNMECRVCGDKASGFHYGVHACEGCKGFFRRTIRMKLEYEKCERS
CKIQKKNRNKCQYCRFQKCLALGMSHNAIRFGRMPEAEKRKLVAGLTANEGSQYNP
QVADLKAFSKHIYNAYLKNFNMTKKKARSILTGKASHTAPFVIHDIETLWQAEKGLV
WKQLVNGLPPYKEISVHVFYRCQCTTVETVRELTEFAKSIPSFSSLFLNDQVTLLKYG
VHEAIFAMLASIVNKDGLLVANGSGFVTREFLRSLRKPFSDIIEPKFEFAVKFNALELD
DSDLALFIAAIILCGDRPGLMNVPRVEAIQDTILRALEFHLQANHPDAQYLFPKLLQK
MADLRQLVTEHAQMMQRIKKTETETSLHPLLQEIYKDMY (SEQ ID NO: 21)
Amino acid sequence encoding a human PPARδ sequence (UniProtKB Accession No. Q03181-2):
MEQPQEEAPEVREEEEKEEVAEAEGAPELNGGPQHALPSSSYTDLSRSSSPPSLLDQL
QMGCDGASCGSLNMECRVCGDKASGFHYGVHACEGCKGFFRRTIRMKLEYEKCER
SCKIQKKNRNKCQYCRFQKCLALGMSHNAIRFGRMPEAEKRKLVAGLTANEGSQYN
PQVADLKAFSKHIYNAYLKNFNMTKKKARSILTGKASHTAPFVIHDIETLWQAEKGL
VWKQLVNGLPPYKEISVHVFYRCQCTTVETVRELTEFAKSIPSFSSLFLNDQVTLLKY
GVHEAIFAMLASIVNKDGLLVANGSGFVTREFLRSLRKPFSDIIEPKFEFAVKFNALEL
DDSDLALFIAAIILCGGE (SEQ ID NO: 14)
Amino acid sequence encoding a human PPARδ sequence (UniProtKB Accession No. Q03181-3):
MHQRDLSRSSSPPSLLDQLQMGCDGASCGSLNMECRVCGDKASGFHYGVHACEGC
KGFFRRTIRMKLEYEKCERSCKIQKKNRNKCQYCRFQKCLALGMSHNAIRFGRMPE
AEKRKLVAGLTANEGSQYNPQVADLKAFSKHIYNAYLKNFNMTKKKARSILTGKAS
HTAPFVIHDIETLWQAEKGLVWKQLVNGLPPYKEISVHVFYRCQCTTVETVRELTEF
AKSIPSFSSLFLNDQVTLLKYGVHEAIFAMLASIVNKDGLLVANGSGFVTREFLRSLR
KPFSDIIEPKFEFAVKFNALELDDSDLALFIAAIILCGDRPGLMNVPRVEAIQDTILRAL
EFHLQANHPDAQYLFPKLLQKMADLRQLVTEHAQMMQRIKKTETETSLHPLLQEIY
KDMY (SEQ ID NO: 15)
Amino acid sequence encoding a human PPARδ sequence (UniProtKB Accession No. Q03181-4):
MEQPQEEAPEVREEEEKEEVAEAEGAPELNGGPQHALPSSSYTAIRFGRMPEAEKRK
LVAGLTANEGSQYNPQVADLKAFSKHIYNAYLKNFNMTKKKARSILTGKASHTAPF
VIHDIETLWQAEKGLVWKQLVNGLPPYKEISVHVFYRCQCTTVETVRELTEFAKSIPS
FSSLFLNDQVTLLKYGVHEAIFAMLASIVNKDGLLVANGSGFVTREFLRSLRKPFSDII
EPKFEFAVKFNALELDDSDLALFIAAIILCGDRPGLMNVPRVEAIQDTILRALEFHLQA
NHPDAQYLFPKLLQKMADLRQLVTEHAQMMQRIKKTETETSLHPLLQEIYKDMY
(SEQ ID NO: 16)
Disordered region (1-54) of a wild-type PPARδ
MEQPQEEAPEVREEEEKEEVAEAEGAPELNGGPQHALPSSSYTDLSRSSSPPSL (SEQ
ID NO: 24)
DNA Binding Domain (71-145) of a wild-type PPARδ
NMECRVCGDKASGFHYGVHACEGCKGFFRRTIRMKLEYEKCERSCKIQKKNRNKC
QYCRFQKCLALGMSHNAIRF (SEQ ID NO: 25)
Ligand Binding Domain (211-439) of a wild-type PPARδ
FVIHDIETLWQAEKGLVWKQLVNGLPPYKEISVHVFYRCQCTTVETVRELTEFAKSIP
SFSSLFLNDQVTLLKYGVHEAIFAMLASIVNKDGLLVANGSGFVTREFLRSLRKPFSD
IIEPKFEFAVKFNALELDDSDLALFIAAIILCGDRPGLMNVPRVEAIQDTILRALEFHLQ
ANHPDAQYLFPKLLQKMADLRQLVTEHAQMMQRIKKTETETSLHPLLQEIYKD
(SEQ ID NO: 26)
Amino acid sequence encoding a mouse PPARδ sequence (UniProtKB Accession No.
P35396):
MEQPQEETPEAREEEKEEVAMGDGAPELNGGPEHTLPSSSCADLSQNSSPSSLLDQL
QMGCDGASGGSLNMECRVCGDKASGFHYGVHACEGCKGFFRRTIRMKLEYEKCDR
ICKIQKKNRNKCQYCRFQKCLALGMSHNAIRFGRMPEAEKRKLVAGLTASEGCQHN
PQLADLKAFSKHIYNAYLKNFNMTKKKARSILTGKSSHNAPFVIHDIETLWQAEKGL
VWKQLVNGLPPYNEISVHVFYRCQSTTVETVRELTEFAKNIPNFSSLFLNDQVTLLKY
GVHEAIFAMLASIVNKDGLLVANGSGFVTHEFLRSLRKPFSDIIEPKFEFAVKFNALEL
DDSDLALFIAAIILCGDRPGLMNVPQVEAIQDTILRALEFHLQVNHPDSQYLFPKLLQ
KMADLRQLVTEHAQMMQWLKKTESETLLHPLLQEIYKDMY (SEQ ID NO: 2)
Nucleic acid sequence encoding a PPARδ (GenBank Accession No. AY919140.1):
ATGGAGCAGCCACAGGAGGAAGCCCCTGAGGTCCGGGAAGAGGAGGAGAAAGA
GGAAGTGGCAGAGGCAGAAGGAGCCCCAGAGCTCAATGGGGGACCACAGCATG
CACTTCCTTCCAGCAGCTACACAGACCTCTCCCGGAGCTCCTCGCCACCCTCACT
GCTGGACCAACTGCAGATGGGCTGTGACGGGGCCTCATGCGGCAGCCTCAACAT
GGAGTGCCGGGTGTGCGGGGACAAGGCATCGGGCTTCCACTACGGTGTTCATGC
ATGTGAGGGGTGCAAGGGCTTCTTCCGTCGTACGATCCGCATGAAGCTGGAGTAC
GAGAAGTGTGAGCGCAGCTGCAAGATTCAGAAGAAGAACCGCAACAAGTGCCA
GTACTGCCGCTTCCAGAAGTGCCTGGCACTGGGCATGTCACACAACGCTATCCGT
TTTGGTCGGATGCCGGAGGCTGAGAAGAGGAAGCTGGTGGCAGGGCTGACTGCA
AATGAGGGGAGCCAGTACAACCCACAGGTGGCCGACCTGAAGGCCTTCTCCAAG
CACATCTACAATGCCTACCTGAAAAACTTCAACATGACCAAAAAGAAGGCCCGC
AGCATCCTCACCGGCAAAGCCAGCCACACGGCGCCCTTTGTGATCCACGACATCG
AGACATTGTGGCAGGCAGAGAAGGGGCTGGTGTGGAAGCAGTTGGTGAATGGCC
TGCCTCCCTACAAGGAGATCAGCGTGCACGTCTTCTACCGCTGCCAGTGCACCAC
AGTGGAGACCGTGCGGGAGCTCACTGAGTTCGCCAAGAGCATCCCCAGCTTCAG
CAGCCTCTTCCTCAACGACCAGGTTACCCTTCTCAAGTATGGCGTGCACGAGGCC
ATCTTCGCCATGCTGGCCTCTATCGTCAACAAGGACGGGCTGCTGGTAGCCAACG
GCAGTGGCTTTGTCACCCGTGAGTTCCTGCGCAGCCTCCGCAAACCCTTCAGTGA
TATCATTGAGCCTAAGTTTGAATTTGCTGTCAAGTTCAACGCCCTGGAACTTGAT
GACAGTGACCTGGCCCTATTCATTGCGGCCATCATTCTGTGTGGAGACCGGCCAG
GCCTCATGAACGTTCCACGGGTGGAGGCTATCCAGGACACCATCCTGCGTGCCCT
CGAATTCCACCTGCAGGCCAACCACCCTGATGCCCAGTACCTCTTCCCCAAGCTG
CTGCAGAAGATGGCTGACCTGCGGCAACTGGTCACCGAGCACGCCCAGATGATG
CAGCGGATCAAGAAGACCGAAACCGAGACCTCGCTGCACCCTCTGCTCCAGGAG
ATCTACAAGGACATGTACTAA (SEQ ID NO: 3)
Nucleic acid sequence encoding a PPARδ
GAGCAGCCACAGGAGGAAGCCCCTGAGGTCCGGGAAGAGGAGGAGAAAGAGGA
AGTGGCAGAGGCAGAAGGAGCCCCAGAGCTCAATGGGGGACCACAGCATGCAC
TTCCTTCCAGCAGCTACACAGACCTCTCCCGGAGCTCCTCGCCACCCTCACTGCT
GGACCAACTGCAGATGGGCTGTGACGGGGCCTCATGCGGCAGCCTCAACATGGA
GTGCCGGGTGTGCGGGGACAAGGCATCGGGCTTCCACTACGGTGTTCATGCATGT
GAGGGGTGCAAGGGCTTCTTCCGTCGTACGATCCGCATGAAGCTGGAGTACGAG
AAGTGTGAGCGCAGCTGCAAGATTCAGAAGAAGAACCGCAACAAGTGCCAGTAC
TGCCGCTTCCAGAAGTGCCTGGCACTGGGCATGTCACACAACGCTATCCGTTTTG
GTCGGATGCCGGAGGCTGAGAAGAGGAAGCTGGTGGCAGGGCTGACTGCAAATG
AGGGGAGCCAGTACAACCCACAGGTGGCCGACCTGAAGGCCTTCTCCAAGCACA
TCTACAATGCCTACCTGAAAAACTTCAACATGACCAAAAAGAAGGCCCGCAGCA
TCCTCACCGGCAAAGCCAGCCACACGGCGCCCTTTGTGATCCACGACATCGAGAC
ATTGTGGCAGGCAGAGAAGGGGCTGGTGTGGAAGCAGTTGGTGAATGGCCTGCC
TCCCTACAAGGAGATCAGCGTGCACGTCTTCTACCGCTGCCAGTGCACCACAGTG
GAGACCGTGCGGGAGCTCACTGAGTTCGCCAAGAGCATCCCCAGCTTCAGCAGC
CTCTTCCTCAACGACCAGGTTACCCTTCTCAAGTATGGCGTGCACGAGGCCATCT
TCGCCATGCTGGCCTCTATCGTCAACAAGGACGGGCTGCTGGTAGCCAACGGCA
GTGGCTTTGTCACCCGTGAGTTCCTGCGCAGCCTCCGCAAACCCTTCAGTGATAT
CATTGAGCCTAAGTTTGAATTTGCTGTCAAGTTCAACGCCCTGGAACTTGATGAC
AGTGACCTGGCCCTATTCATTGCGGCCATCATTCTGTGTGGAGACCGGCCAGGCC
TCATGAACGTTCCACGGGTGGAGGCTATCCAGGACACCATCCTGCGTGCCCTCGA
ATTCCACCTGCAGGCCAACCACCCTGATGCCCAGTACCTCTTCCCCAAGCTGCTG
CAGAAGATGGCTGACCTGCGGCAACTGGTCACCGAGCACGCCCAGATGATGCAG
CGGATCAAGAAGACCGAAACCGAGACCTCGCTGCACCCTCTGCTCCAGGAGATC
TACAAGGACATGTAC (SEQ ID NO: 19)
Nucleic acid sequence encoding a PPARδ (GenBank Accession No. NM_006238.5):
GCGGAGCGTGTGACGCTGCGGCCGCCGCGGACCTGGGGATTAATGGGAAAAGTT
TTGGCAGGAGCGGGAGAATTCTGCGGAGCCTGCGGGACGGCGGCGGTGGCGCCG
TAGGCAGCCGGGACAGTGTTGTACAGTGTTTTGGGCATGCACGTGATACTCACAC
AGTGGCTTCTGCTCACCAACAGATGAAGACAGATGCACCAACGAGGCTGATGGG
AACCACCCTGTAGAGGTCCATCTGCGTTCAGACCCAGACGATGCCAGAGCTATG
ACTGGGCCTGCAGGTGTGGCGCCGAGGGGAGATCAGCCATGGAGCAGCCACAGG
AGGAAGCCCCTGAGGTCCGGGAAGAGGAGGAGAAAGAGGAAGTGGCAGAGGCA
GAAGGAGCCCCAGAGCTCAATGGGGGACCACAGCATGCACTTCCTTCCAGCAGC
TACACAGACCTCTCCCGGAGCTCCTCGCCACCCTCACTGCTGGACCAACTGCAGA
TGGGCTGTGACGGGGCCTCATGCGGCAGCCTCAACATGGAGTGCCGGGTGTGCG
GGGACAAGGCATCGGGCTTCCACTACGGTGTTCATGCATGTGAGGGGTGCAAGG
GCTTCTTCCGTCGTACGATCCGCATGAAGCTGGAGTACGAGAAGTGTGAGCGCA
GCTGCAAGATTCAGAAGAAGAACCGCAACAAGTGCCAGTACTGCCGCTTCCAGA
AGTGCCTGGCACTGGGCATGTCACACAACGCTATCCGTTTTGGTCGGATGCCGGA
GGCTGAGAAGAGGAAGCTGGTGGCAGGGCTGACTGCAAACGAGGGGAGCCAGT
ACAACCCACAGGTGGCCGACCTGAAGGCCTTCTCCAAGCACATCTACAATGCCTA
CCTGAAAAACTTCAACATGACCAAAAAGAAGGCCCGCAGCATCCTCACCGGCAA
AGCCAGCCACACGGCGCCCTTTGTGATCCACGACATCGAGACATTGTGGCAGGC
AGAGAAGGGGCTGGTGTGGAAGCAGTTGGTGAATGGCCTGCCTCCCTACAAGGA
GATCAGCGTGCACGTCTTCTACCGCTGCCAGTGCACCACAGTGGAGACCGTGCGG
GAGCTCACTGAGTTCGCCAAGAGCATCCCCAGCTTCAGCAGCCTCTTCCTCAACG
ACCAGGTTACCCTTCTCAAGTATGGCGTGCACGAGGCCATCTTCGCCATGCTGGC
CTCTATCGTCAACAAGGACGGGCTGCTGGTAGCCAACGGCAGTGGCTTTGTCACC
CGTGAGTTCCTGCGCAGCCTCCGCAAACCCTTCAGTGATATCATTGAGCCTAAGT
TTGAATTTGCTGTCAAGTTCAACGCCCTGGAACTTGATGACAGTGACCTGGCCCT
ATTCATTGCGGCCATCATTCTGTGTGGAGACCGGCCAGGCCTCATGAACGTTCCA
CGGGTGGAGGCTATCCAGGACACCATCCTGCGTGCCCTCGAATTCCACCTGCAGG
CCAACCACCCTGATGCCCAGTACCTCTTCCCCAAGCTGCTGCAGAAGATGGCTGA
CCTGCGGCAACTGGTCACCGAGCACGCCCAGATGATGCAGCGGATCAAGAAGAC
CGAAACCGAGACCTCGCTGCACCCTCTGCTCCAGGAGATCTACAAGGACATGTA
CTAACGGCGGCACCCAGGCCTCCCTGCAGACTCCAATGGGGCCAGCACTGGAGG
GGCCCACCCACATGACTTTTCCATTGACCAGCCCTTGAGCACCCGGCCTGGAGCA
GCAGAGTCCCACGATCGCCCTCAGACACATGACACCCACGGCCTCTGGCTCCCTG
TGCCCTCTCTCCCGCTTCCTCCAGCCAGCTCTCTTCCTGTCTTTGTTGTCTCCCTCT
TTCTCAGTTCCTCTTTCTTTTCTAATTCCTGTTGCTCTGTTTCTTCCTTTCTGTAGGT
TTCTCTCTTCCCTTCTCCCTTGCCCTCCCTTTCTCTCTCCACCCCCCACGTCTGTCC
TCCTTTCTTATTCTGTGAGATGTTTTGTATTATTTCACCAGCAGCATAGAACAGGA
CCTCTGCTTTTGCACACCTTTTCCCCAGGAGCAGAAGAGAGTGGGGCCTGCCCTC
TGCCCCATCATTGCACCTGCAGGCTTAGGTCCTCACTTCTGTCTCCTGTCTTCAGA
GCAAAAGACTTGAGCCATCCAAAGAAACACTAAGCTCTCTGGGCCTGGGTTCCA
GGGAAGGCTAAGCATGGCCTGGACTGACTGCAGCCCCCTATAGTCATGGGGTCC
CTGCTGCAAAGGACAGTGGGCAGGAGGCCCCAGGCTGAGAGCCAGATGCCTCCC
CAAGACTGTCATTGCCCCTCCGATGCTGAGGCCACCCACTGACCCAACTGATCCT
GCTCCAGCAGCACACCTCAGCCCCACTGACACCCAGTGTCCTTCCATCTTCACAC
TGGTTTGCCAGGCCAATGTTGCTGATGGCCCCCTGCACTGGCCGCTGGACGGCAC
TCTCCCAGCTTGGAAGTAGGCAGGGTTCCCTCCAGGTGGGCCCCCACCTCACTGA
AGAGGAGCAAGTCTCAAGAGAAGGAGGGGGGATTGGTGGTTGGAGGAAGCAGC
ACACCCAATTCTGCCCCTAGGACTCGGGGTCTGAGTCCTGGGGTCAGGCCAGGG
AGAGCTCGGGGCAGGCCTTCCGCCAGCACTCCCACTGCCCCCCTGCCCAGTAGCA
GCCGCCCACATTGTGTCAGCATCCAGGGCCAGGGCCTGGCCTCACATCCCCCTGC
TCCTTTCTCTAGCTGGCTCCACGGGAGTTCAGGCCCCACTCCCCCTGAAGCTGCC
CCTCCAGCACACACACATAAGCACTGAAATCACTTTACCTGCAGGCTCCATGCAC
CTCCCTTCCCTCCCTGAGGCAGGTGAGAACCCAGAGAGAGGGGCCTGCAGGTGA
GCAGGCAGGGCTGGGCCAGGTCTCCGGGGAGGCAGGGGTCCTGCAGGTCCTGGT
GGGTCAGCCCAGCACCTGCTCCCAGTGGGAGCTTCCCGGGATAAACTGAGCCTGT
TCATTCTGATGTCCATTTGTCCCAATAGCTCTACTGCCCTCCCCTTCCCCTTTACTC
AGCCCAGCTGGCCACCTAGAAGTCTCCCTGCACAGCCTCTAGTGTCCGGGGACCT
TGTGGGACCAGTCCCACACCGCTGGTCCCTGCCCTCCCCTGCTCCCAGGTTGAGG
TGCGCTCACCTCAGAGCAGGGCCAAAGCACAGCTGGGCATGCCATGTCTGAGCG
GCGCAGAGCCCTCCAGGCCTGCAGGGGCAAGGGGCTGGCTGGAGTCTCAGAGCA
CAGAGGTAGGAGAACTGGGGTTCAAGCCCAGGCTTCCTGGGTCCTGCCTGGTCCT
CCCTCCCAAGGAGCCATTCTGTGTGTGACTCTGGGTGGAAGTGCCCAGCCCCTGC
CCCTACGGGCGCTGCAGCCTCCCTTCCATGCCCCAGGATCACTCTCTGCTGGCAG
GATTCTTCCCGCTCCCCACCTACCCAGCTGATGGGGGTTGGGGTGCTTCCTTTCAG
GCCAAGGCTATGAAGGGACAGCTGCTGGGACCCACCTCCCCCTCCCCGGCCACA
TGCCGCGTCCCTGCCCCGACCCGGGTCTGGTGCTGAGGATACAGCTCTTCTCAGT
GTCTGAACAATCTCCAAAATTGAAATGTATATTTTTGCTAGGAGCCCCAGCTTCC
TGTGTTTTTAATATAAATAGTGTACACAGACTGACGAAACTTTAAATAAATGGGA
ATTAAATATTTAA (SEQ ID NO: 17)
G4S linker
GGGGS (SEQ ID NO: 4)
(G4S)₂ linker
GGGGSGGGGS (SEQ ID NO: 32)
(G4S)₃ linker
GGGGSGGGGSGGGGS (SEQ ID NO: 33)
(G4S)₄ linker
GGGGSGGGGSGGGGSGGGGS (SEQ ID NO: 34)
EAAAK linker
EAAAK (SEQ ID NO: 35)
(EAAAK)₂ linker
EAAAKEAAAK (SEQ ID NO: 36)
(EAAAK)₃ linker
EAAAKEAAAKEAAAK (SEQ ID NO: 37)
PAPAP linker
PAPAP (SEQ ID NO: 38)
AEAAAKEAAAKA linker
AEAAAKEAAAKA (SEQ ID NO: 39)
Rat CPT1AM (active mutant of carnitine palmitoyltransferase-1a) sequence [2322 bp total]:
ATGGCAGAGGCTCACCAAGCTGTGGCCTTCCAGTTCACCGTCACCCCCGATGGCA
TTGACCTCCGCCTGAGCCACGAAGCCCTCAAACAGATCTGCCTGTCGGGGCTGCA
CTCCTGGAAGAAGAAGTTCATCCGGTTCAAGAATGGCATCATCACTGGTGTGTTC
CCCGCGAATCCGTCCAGCTGGCTTATCGTGGTGGTGGGTGTGATTTCATCCATGC
ATGCCAAAGTGGACCCCTCCCTGGGCATGATCGCAAAGATCAGTCGGACCCTAG
ACACCACTGGCCGCATGTCAAGCCAGACGAAGAACATTGTGAGCGGCGTCCTCT
TTGGTACAGGGCTCTGGGTGGCAGTCATCATGACCATGCGCTACTCGCTGAAGGT
GCTGCTCTCCTACCACGGCTGGATGTTTGCAGAACACGGCAAAATGAGCCGCAG
CACCAAGATCTGGATGGCTATGGTCAAGGTCCTCTCAGGTCGGAAGCCCATGTTG
TACAGCTTCCAGACGTCTCTGCCACGCCTGCCTGTCCCAGCTGTCAAAGATACTG
TGAGCAGGTACCTGGAATCTATGAGGCCACTGATGAAGGAAGAAGACTTCCAGC
GCATGACAGCACTGGCCCAGGATTTTGCTGTCAACCTCGGACCCAAATTGCAGTG
GTATTTGAAGCTAAAATCCTGGTGGGCCACAAATTACGTGAGTGACTGGTGGGA
AGAATATATCTACCTGCGGGGCCGAGGGCCGCTCATGGTCAACAGCAACTACTA
CGCCATGGAGATGCTGTACATCACCCCAACCCATATCCAGGCAGCGAGAGCTGG
CAACACCATCCACGCCATACTGCTGTATCGTCGCACATTAGACCGTGAGGAACTC
AAACCCATTCGTCTTCTGGGATCCACCATTCCACTCTGCTCAGCCCAGTGGGAGC
GACTCTTCAATACTTCCCGGATCCCTGGGGAGGAGACAGACACCATCCAACATAT
CAAGGACAGCAGGCACATTGTTGTGTACCACAGAGGGCGGTACTTCAAGGTCTG
GCTCTACCACGATGGGAGGCTGCTGAGGCCCCGAGAGCTGGAGCAGCAAATGCA
GCAGATCCTGGATGATCCCTCAGAGCCACAGCCTGGGGAGGCCAAGCTGGCCGC
CCTCACTGCTGCAGACAGAGTGCCCTGGGCAAAGTGTCGGCAGACCTATTTTGCA
CGAGGGAAAAATAAGCAGTCCCTGGATGCGGTGGAAAAGGCAGCGTTCTTCGTG
ACGTTGGACGAATCGGAGCAGGGATACAGAGAGGAGGATCCTGAGGCATCCATC
GACAGCTACGCCAAATCCCTGCTGCATGGAAGATGCTTTGACAGGTGGTTTGACA
AGTCCATCACCTTTGTTGTCTTCAAAAACAGCAAGATAGGCATAAATGCAGAGCA
CTCCTGGGCGGACGCGCCCATCGTGGGCCATTTGTGGGAGTATGTCATGGCCACC
GACGTCTTCCAGCTGGGTTACTCAGAGGATGGACACTGTAAAGGAGACACCAAC
CCCAACATCCCTAAGCCCACAAGGCTACAATGGGACATTCCAGGAGAGTGCCAG
GAGGTCATAGATGCATCCCTGAGCAGCGCCAGTCTTTTGGCAAATGATGTGGACC
TGCATTCCTTCCCATTTGACTCTTTCGGCAAAGGCTTGATCAAGAAGTGCCGGAC
GAGTCCCGATGCCTTCATCCAGCTGGCGCTGCAGCTCGCACATTACAAGGACATG
GGCAAGTTCTGCCTCACATATGAGGCCTCCAGTACCCGGCTCTTCCGAGAAGGGA
GGACAGAGACTGTACGCTCCTGCACTATGGAGTCCTGCAACTTTGTGCAGGCCAT
GATGGACCCCAAGTCAACGGCAGAGCAGAGGCTCAAGCTGTTCAAGATAGCTTG
TGAGAAGCACCAGCACCTGTACCGCCTCGCCATGACGGGCGCCGGCATCGACCG
CCATCTCTTCTGCCTCTATGTGGTGTCCAAGTATCTTGCAGTCGACTCACCTTTCC
TGAAGGAGGTATTGTCTGAGCCATGGAGGTTGTCTACGAGCCAGACTCCTCAGCA
GCAGGTGGAGCTCTTTGACTTTGAGAAAAACCCTGACTATGTGTCCTGTGGAGGG
GGCTTTGGGCCGGTTGCCGATGACGGCTATGGTGTCTCCTACATTATAGTGGGAG
AGAATTTTATCCACTTCCATATTTCTTCCAAGTTCTCTAGCCCTGAGACAGACTCA
CACCGCTTTGGGAAGCACTTGAGACAAGCCATGATGGACATTATCACCTTGTTTG
GCCTCACCATCAATTCTAAAAAGTAA (SEQ ID NO: 5)
VP16 Sequence:
DALDDFDLDML (SEQ ID NO: 6)
VP64 Sequence:
DALDDFDLDMLGSDALDDFDLDMLGSDALDDFDLDMLGSDALDDFDLDML (SEQ ID NO: 7)
Key for SEQ ID NO: 7:
GS linkers are underlined
DALDDFDLDML (SEQ ID NO: 6) corresponds to amino acids 437-447 of Herpes Simplex
Viral Protein 16
Nucleotide sequence encoding VP64:
gatgctttagacgattttgacttagatatgcttggttcagacgcgttagacgacttcgacctagacatgttaggctcagatgcattggacga
cttcgatttagatatgttgggctccgatgccctagatgacttcgacctggacatgctg (SEQ ID NO: 8)
(Gly)₆ linker:
GGGGGG (SEQ ID NO: 9)
(Gly)₈ linker:
GGGGGGGG (SEQ ID NO: 10)
A helical (H4)₂ linker:
A(EAAAK)₄ALEA(EAAAK)₄A (SEQ ID NO: 11)
Nucleic acid sequence encoding a linker:
ggaggaggaggtagt (SEQ ID NO: 18)
Amino acid sequence encoding human CPT1A (UniProt Accession No. P50416):
MAEAHQAVAFQFTVTPDGIDLRLSHEALRQIYLSGLHSWKKKFIRFKNGIITGVYPAS
PSSWLIVVVGVMTTMYAKIDPSLGIIAKINRTLETANCMSSQTKNVVSGVLFGTGLW
VALIVTMRYSLKVLLSYHGWMFTEHGKMSRATKIWMGMVKIFSGRKPMLYSFQTS
LPRLPVPAVKDTVNRYLQSVRPLMKEEDFKRMTALAQDFAVGLGPRLQWYLKLKS
WWATNYVSDWWEEYIYLRGRGPLMVNSNYYAMDLLYILPTHIQAARAGNAIHAIL
LYRRKLDREEIKPIRLLGSTIPLCSAQWERMENTSRIPGEETDTIQHMRDSKHIVVYHR
GRYFKVWLYHDGRLLKPREMEQQMQRILDNTSEPQPGEARLAALTAGDRVPWARC
RQAYFGRGKNKQSLDAVEKAAFFVTLDETEEGYRSEDPDTSMDSYAKSLLHGRCYD
RWFDKSFTFVVFKNGKMGLNAEHSWADAPIVAHLWEYVMSIDSLQLGYAEDGHCK
GDINPNIPYPTRLQWDIPGECQEVIETSLNTANLLANDVDFHSFPFVAFGKGIIKKCRT
SPDAFVQLALQLAHYKDMGKFCLTYEASMTRLFREGRTETVRSCTTESCDFVRAMV
DPAQTVEQRLKLFKLASEKHQHMYRLAMTGSGIDRHLFCLYVVSKYLAVESPFLKE
VLSEPWRLSTSQTPQQQVELFDLENNPEYVSSGGGFGPVADDGYGVSYILVGENLIN
FHISSKFSCPETDSHRFGRHLKEAMTDIITLFGLSSNSKK (SEQ ID NO: 12)
Nucleic acid sequence encoding human CPTIA (NCBI Reference Sequence: NM_001876.4):
aatccgctgctgccggcgtcgggtgcgctcggcctcgcccgcggccctccttccccggctcccgctcgccgctcgttcactccaccg
ccgccgccgccgccgccgctgccgctgccgctgccgcacctccgtagctgactcggtactctctgaagatggcagaagctcaccaa
gctgtggcctttcagttcacggtcactccggacgggattgacctgcggctgagccatgaagctcttagacaaatctatctctctggacttc
attcctggaaaaagaagttcatcagattcaagaacggcatcatcactggcgtgtacccggcaagcccctccagttggcttatcgtggtg
gtgggcgtgatgacaacgatgtacgccaagatcgacccctcgttaggaataattgcaaaaatcaatcggactctggaaacggccaact
gcatgtccagccagacgaagaacgtggtcagcggcgtgctgtttggcaccggcctgtgggtggccctcatcgtcaccatgcgctact
ccctgaaagtgctgctctcctaccacgggtggatgttcactgagcacggcaagatgagtcgtgccaccaagatctggatgggtatggt
caagatcttttcaggccgaaaacccatgttgtacagcttccagacatcgctgcctcgcctgccggtcccggctgtcaaagacactgtga
acaggtatctacagtcggtgaggcctcttatgaaggaagaagacttcaaacggatgacagcacttgctcaagattttgctgtcggtcttg
gaccaagattacagtggtatttgaagttaaaatcctggtgggctacaaattacgtgagcgactggtgggaggagtacatctacctccga
ggacgagggccgctcatggtgaacagcaactattatgccatggatctgctgtatatccttccaactcacattcaggcagcaagagccg
gcaacgccatccatgccatcctgctttacaggcgcaaactggaccgggaggaaatcaaaccaattcgtcttttgggatccacgattcca
ctctgctccgctcagtgggagcggatgtttaatacttcccggatcccaggagaggagacagacaccatccagcacatgagagacagc
aagcacatcgtcgtgtaccatcgaggacgctacttcaaggtctggctctaccatgatgggcggctgctgaagccccgggagatggag
cagcagatgcagaggatcctggacaatacctcggagcctcagcccggggaggccaggctggcagccctcaccgcaggagacaga
gttccctgggccaggtgtcgtcaggcctattttggacgtgggaaaaataagcagtctcttgatgctgtggagaaagcagcgttctttgtg
acgttagatgaaactgaagaaggatacagaagtgaagacccggatacgtcaatggacagctacgccaaatctctactacacggccga
tgttacgacaggtggtttgacaagtcgttcacgtttgttgtcttcaaaaacgggaagatgggcctcaacgctgaacactcctgggcagat
gcgccgatcgtggcccacctttgggagtacgtcatgtccattgacagcctccagctgggctatgcggaggatgggcactgcaaaggc
gacatcaatccgaacattccgtaccccaccaggctgcagtgggacatcccgggggaatgtcaagaggttatagagacctccctgaac
accgcaaatcttctggcaaacgacgtggatttccattccttcccattcgtagcctttggtaaaggaatcatcaagaaatgtcgcacgagcc
cagacgcctttgtgcagctggccctccagctggcgcactacaaggacatgggcaagttttgcctcacatacgaggcctccatgacccg
gctcttccgagaggggaggacggagaccgtgcgctcctgcaccactgagtcatgcgacttcgtggggccatggtggacccggccc
agacggtggaacagaggctgaagttgttcaagttggcgtctgagaagcatcagcatatgtatcgcctcgccatgaccggctctgggat
cgatcgtcacctcttctgcctttacgtggtgtctaaatatctcgctgtggagtcccctttccttaaggaagttttatctgagccttggagat
tatcaacaagccagacccctcagcagcaagtggagctgtttgacttggagaataacccagagtacgtgtccagcggagggggctttgga
ccggttgctgatgacggctatggtgtgtcgtacatccttgtgggagagaacctcatcaatttccacatttcttccaagttctcttgccctgag
acggattctcatcgctttggaaggcacctgaaagaagcaatgactgacatcatcactttgtttggtctcagttctaattccaaaaagtaattc
cactggagctgctgggaaggaaaacgagctcttctgatgcaaaccaaatgaaaaataggcattaatcctgaccttagctcgggatgaa
acactgctcttaaaaaaactcagttttccttccagaaaatgtgggtgtttttttttcctagaacagtatctctcccctgtgaagcataacccc
actacttccagacttgccctcccttgggggacatctgataaagtctcccctgatgtctccgcatcggcttggatttattaagggatgcaaatc
ttgttgagttaatgaaggaattagtagggttgtggcttcacacacagtggaatggaaatggtgtgctttctcagtggcaaccgaaggcct
agtgcttaagggcatttagcatcatccaagcagggtaaacttttgttttgttaaaagaaaaatgtgttattcaagttggtgtccccagttgta
gctaacacatctggaatgcactaaccaaaatgctgtgctttggagacctgcttttgtcaccgtgggtaaccgttcccgtctggtccagtag
cctgtgtttgcctctccacatttgaagcaagcaggatgcaaggtcttcagttttactgaccttgtatgtcttcaagtcttcacaacccagtgc
cttaaaaatgaaaggccctaaatgtaagggagatggagagaaagatttattttgtagagtctttgggtggaattgtgggtatactgttccct
tcacaattgactgagtatggataaccgtacataagcatttgctacaccccaccagccccctccccctcagaaacaccagttccttcccaa
gggcagctgtgccagactcccctcccgggactgccttcttgtcatcataagcaacaaaagaaataacaggcacatgtcataaaagggg
agcaagggccgtgatggtcagataattcactcaagaataaaacatgacacgtgcctcaggaggatctctttcccaaagtgacagcaag
gagggcagggcatcggccaccaagcggggactagcaagtgaggaaggggagggcagcccaccgtggtgaggagagagtggct
ccacgaccccaagggatggccttctcctcccacccggtgaggggaaagactcaccagagggtgatggagacagtatgccggctca
ccttggtgaccagccaagatgtctcaagtgacagtgctaggtgttcacccagcctgtccttcagataggagtgccttcacgaaagcgtct
catggaccacaaagcaattatgcactgagtcatcttcagtatttaatgcaaaaatgaagcatcatggaatgaaattcccactgtctgtcatg
acaagcttagctgtccattgttttaaattgtgtatttatttttttgaccacttggttctagttgggcctgactccttcagagtgctgcacccc
gatagtacaacagcgatggctgaactgttggagtcgatggaaggtgcttgccggagaacacgtgcctttttttttttttttttttttgagatg
gagtttcactcttgttgcccaggctggagtgcagtggtgcaatctcggctcactgcagcatctgccttgcaggttcaagcgattctcctgtct
cagcctcccaagtagctgggattacacgcccaacaccatgccctgctaatttttgtatttttagtagagacggggtttcatcatattggtcag
gctggtctcgaactcctgacctcaggtgatccacctgcctcagcctcccaaagtgctgggattacaggcatgagccaccatgcgcggcc
cacatgcatgttttatgtatgtatacttcatgatgtaaaaatcccacctttatgggccaaagattttttttctcctgaaagcaagaaaaaatg
aaaacaaaagacaaaaaaaaaaaaaaaagcgtccaggcgcggtggctcatgcgtgtaatcccagcactttgggaggccaaggggg
cggatcacgaggtcaggagatggagaccatcctggctaacacggtgaaaccccgtctctactaaaaattcaaaaaatgagccgggcg
tggtggcgggcgcctgtagtcccagctactcaggaggctgaggcaggagaatggcgtgaacccgggaggcggagcttgcagtga
gccgagatcgctccactgcactccagcctgggcgacagagcgagactccgtctaaaaaaaaaagcaaaaacaaaccaacaacaaa
agcccctgactgtccgtcaagcaggcagcggggatgtagctctctctgccctgggcaagaatagcacttcccgttaaaagccagcag
ccggcgtcagtccctatcagagccagctagatcatgcactgttgaccactgagcaatctgtgttacactagagttcacagggcattttga
gtgtagacgtgagtgcttaaacatatttgggtttctctctcaggttttaaatgtttcaaatgtaattgttgctcatcagtgcagttatcaatg
caattttatattccttgaggggagaaagaggggtcttattgtacatgtccaaggggggtgataagagtattatctgtttaatttaattggaac
aaaccattgtcttaacgcagccatggtttgaatttgttatcttgggctgaccggtgcatgtaaatacagtatgctctttggatgtaaatctta
gaaatgcagtgtgaatgtaggttatcattaataaaacattaaccccagtctactacaa (SEQ ID NO: 27)
Amino acid sequence encoding RAT CPT1AM:
MAEAHQAVAFQFTVTPDGIDLRLSHEALKQICLSGLHSWKKKFIRFKNGIITGVFPAN
PSSWLIVVVGVISSMHAKVDPSLGMIAKISRTLDTTGRMSSQTKNIVSGVLFGTGLW
VAVIMTMRYSLKVLLSYHGWMFAEHGKMSRSTKIWMAMVKVLSGRKPMLYSFQT
SLPRLPVPAVKDTVSRYLESMRPLMKEEDFQRMTALAQDFAVNLGPKLQWYLKLKS
WWATNYVSDWWEEYIYLRGRGPLMVNSNYYAMEMLYITPTHIQAARAGNTIHAIL
LYRRTLDREELKPIRLLGSTIPLCSAQWERLFNTSRIPGEETDTIQHIKDSRHIVVYHRG
RYFKVWLYHDGRLLRPRELEQQMQQILDDPSEPQPGEAKLAALTAADRVPWAKCR
QTYFARGKNKQSLDAVEKAAFFVTLDESEQGYREEDPEASIDSYAKSLLHGRCFDR
WFDKSITFVVFKNSKIGINAEHSWADAPIVGHLWEYVMATDVFQLGYSEDGHCKGD
TNPNIPKPTRLQWDIPGECQEVIDASLSSASLLANDVDLHSFPFDSFGKGLIKKCRTSP
DAFIQLALQLAHYKDMGKFCLTYEASSTRLFREGRTETVRSCTMESCNFVQAMMDP
KSTAEQRLKLFKIACEKHQHLYRLAMTGAGIDRHLFCLYVVSKYLAVDSPFLKEVLS
EPWRLSTSQTPQQQVELFDFEKNPDYVSCGGGFGPVADDGYGVSYIIVGENFIHFHIS
SKFSSPETDSHRFGKHLRQAMMDIITLFGLTINSKK (SEQ ID NO: 13)
Amino acid sequence of Rat CPT1A Wild-type Sequence (based on active mutant described
in Morillas et al., J Biol Chem. 2003 March 14; 278(11): 9058-63):
MAEAHQAVAFQFTVTPDGIDLRLSHEALKQICLSGLHSWKKKFIRFKNGIITGVFPAN
PSSWLIVVVGVISSMHAKVDPSLGMIAKISRTLDTTGRMSSQTKNIVSGVLFGTGLW
VAVIMTMRYSLKVLLSYHGWMFAEHGKMSRSTKIWMAMVKVLSGRKPMLYSFQT
SLPRLPVPAVKDTVSRYLESMRPLMKEEDFQRMTALAQDFAVNLGPKLQWYLKLKS
WWATNYVSDWWEEYIYLRGRGPLMVNSNYYAMEMLYITPTHIQAARAGNTIHAIL
LYRRTLDREELKPIRLLGSTIPLCSAQWERLFNTSRIPGEETDTIQHIKDSRHIVVYHRG
RYFKVWLYHDGRLLRPRELEQQMQQILDDPSEPQPGEAKLAALTAADRVPWAKCR
QTYFARGKNKQSLDAVEKAAFFVTLDESEQGYREEDPEASIDSYAKSLLHGRCFDR
WFDKSITFVVFKNSKIGINAEHSWADAPIVGHLWEYVMATDVFQLGYSEDGHCKGD
TNPNIPKPTRLQWDIPGECQEVIDASLSSASLLANDVDLHSFPFDSFGKGLIKKCRTSP
DAFIQLALQLAHYKDMGKFCLTYEASMTRLFREGRTETVRSCTMESCNFVQAMMDP
KSTAEQRLKLFKIACEKHQHLYRLAMTGAGIDRHLFCLYVVSKYLAVDSPFLKEVLS
EPWRLSTSQTPQQQVELFDFEKNPDYVSCGGGFGPVADDGYGVSYIIVGENFIHFHIS
SKFSSPETDSHRFGKHLRQAMMDIITLFGLTINSKK (SEQ ID NO: 28)
Amino acid sequence of Rat CPT1A Wild type Sequence (UniProt Accession No.: P32198 •
CPT1A_RAT)
MAEAHQAVAFQFTVTPDGIDLRLSHEALKQICLSGLHSWKKKFIRFKNGIITGVFPAN
PSSWLIVVVGVISSMHAKVDPSLGMIAKISRTLDTTGRMSSQTKNIVSGVLFGTGLW
VAVIMTMRYSLKVLLSYHGWMFAEHGKMSRSTKIWMAMVKVLSGRKPMLYSFQT
SLPRLPVPAVKDTVSRYLESVRPLMKEEDFQRMTALAQDFAVNLGPKLQWYLKLKS
WWATNYVSDWWEEYIYLRGRGPLMVNSNYYAMEMLYITPTHIQAARAGNTIHAIL
LYRRTLDREELKPIRLLGSTIPLCSAQWERLFNTSRIPGEETDTIQHIKDSRHIVVYHRG
RYFKVWLYHDGRLLRPRELEQQMQQILDDPSEPQPGEAKLAALTAADRVPWAKCR
QTYFARGKNKQSLDAVEKAAFFVTLDESEQGYREEDPEASIDSYAKSLLHGRCFDR
WFDKSITFVVFKNSKIGINAEHSWADAPVVGHLWEYVMATDVFQLGYSEDGHCKG
DTNPNIPKPTRLQWDIPGECQEVIDASLSSASLLANDVDLHSFPFDSFGKGLIKKCRTS
PDAFIQLALQLAHYKDMGKFCLTYEASMTRLFREGRTETVRSCTMESCNFVQAMMD
PKSTAEQRLKLFKIACEKHQHLYRLAMTGAGIDRHLFCLYVVSKYLAVDSPFLKEVL
SEPWRLSTSQTPQQQVELFDFEKNPDYVSCGGGFGPVADDGYGVSYIIVGENFIHFHI
SSKFSSPETDSHRFGKHLRQAMMDIITLFGLTINSKK (SEQ ID NO: 29)
A nucleic acid sequence encoding a VP64-PPARδ fusion protein (ATG-VP64-linker-PPAR
delta-Flag Tag-Stop codon)
ATGgatgctttagacgattttgacttagatatgcttggttcagacgcgttagacgacttcgacctagacatgttaggctcagat
gcattggacgacttcgatttagatatgttgggctccgatgccctagatgacttcgacctggacatgctgggaggaggaggtagt
ATGGAGCAGCCACAGGAGGAAGCCCCTGAGGTCCGGGAAGAGGAGGAGAAAGAGGA
AGTGGCAGAGGCAGAAGGAGCCCCAGAGCTCAATGGGGGACCACAGCATGCACTTCC
TTCCAGCAGCTACACAGACCTCTCCCGGAGCTCCTCGCCACCCTCACTGCTGGACCAA
CTGCAGATGGGCTGTGACGGGGCCTCATGCGGCAGCCTCAACATGGAGTGCCGGGT
GTGCGGGGACAAGGCATCGGGCTTCCACTACGGTGTTCATGCATGTGAGGGGTGCAA
GGGCTTCTTCCGTCGTACGATCCGCATGAAGCTGGAGTACGAGAAGTGTGAGCGCAG
CTGCAAGATTCAGAAGAAGAACCGCAACAAGTGCCAGTACTGCCGCTTCCAGAAGTGC
CTGGCACTGGGCATGTCACACAACGCTATCCGTTTTGGTCGGATGCCGGAGGCTGAG
AAGAGGAAGCTGGTGGCAGGGCTGACTGCAAATGAGGGGAGCCAGTACAACCCACAG
GTGGCCGACCTGAAGGCCTTCTCCAAGCACATCTACAATGCCTACCTGAAAAACTTCAA
CATGACCAAAAAGAAGGCCCGCAGCATCCTCACCGGCAAAGCCAGCCACACGGCGCC
CTTTGTGATCCACGACATCGAGACATTGTGGCAGGCAGAGAAGGGGCTGGTGTGGAA
GCAGTTGGTGAATGGCCTGCCTCCCTACAAGGAGATCAGCGTGCACGTCTTCTACCGC
TGCCAGTGCACCACAGTGGAGACCGTGCGGGAGCTCACTGAGTTCGCCAAGAGCATC
CCCAGCTTCAGCAGCCTCTTCCTCAACGACCAGGTTACCCTTCTCAAGTATGGCGTGC
ACGAGGCCATCTTCGCCATGCTGGCCTCTATCGTCAACAAGGACGGGCTGCTGGTAG
CCAACGGCAGTGGCTTTGTCACCCGTGAGTTCCTGCGCAGCCTCCGCAAACCCTTCAG
TGATATCATTGAGCCTAAGTTTGAATTTGCTGTCAAGTTCAACGCCCTGGAACTTGATG
ACAGTGACCTGGCCCTATTCATTGCGGCCATCATTCTGTGTGGAGACCGGCCAGGCCT
CATGAACGTTCCACGGGTGGAGGCTATCCAGGACACCATCCTGCGTGCCCTCGAATTC
CACCTGCAGGCCAACCACCCTGATGCCCAGTACCTCTTCCCCAAGCTGCTGCAGAAGA
TGGCTGACCTGCGGCAACTGGTCACCGAGCACGCCCAGATGATGCAGCGGATCAAGA
AGACCGAAACCGAGACCTCGCTGCACCCTCTGCTCCAGGAGATCTACAAGGACATGTA
CgattataaagatgatgatgataaaTAATAGTGA (SEQ ID NO: 20
Key for SEQ ID NO 20:
VP64 sequence (lower case font, bold, and underlined)
linker sequence (lower case font, bold, and italicized)
PPARδ SEQUENCE (CAPITALIZED FONT AND ITALICIZED)
flag tag sequence (lower case font and underlined)
A nucleic acid sequence encoding a VP64-PPAR8 fusion protein (ATG-VP64-linker-PPAR
delta-Stop codon)
ATGgatgctttagacgattttgacttagatatgcttggttcagacgcgttagacgacttcgacctagacatgttaggctcagat
gcattggacgacttcgatttagatatgttgggctccgatgccctagatgacttcgacctggacatgctgggaggaggaggtagt
ATGGAGCAGCCACAGGAGGAAGCCCCTGAGGTCCGGGAAGAGGAGGAGAAAGAGGA
AGTGGCAGAGGCAGAAGGAGCCCCAGAGCTCAATGGGGGACCACAGCATGCACTTCC
TTCCAGCAGCTACACAGACCTCTCCCGGAGCTCCTCGCCACCCTCACTGCTGGACCAA
CTGCAGATGGGCTGTGACGGGGCCTCATGCGGCAGCCTCAACATGGAGTGCCGGGT
GTGCGGGGACAAGGCATCGGGCTTCCACTACGGTGTTCATGCATGTGAGGGGTGCAA
GGGCTTCTTCCGTCGTACGATCCGCATGAAGCTGGAGTACGAGAAGTGTGAGCGCAG
CTGCAAGATTCAGAAGAAGAACCGCAACAAGTGCCAGTACTGCCGCTTCCAGAAGTGC
CTGGCACTGGGCATGTCACACAACGCTATCCGTTTTGGTCGGATGCCGGAGGCTGAG
AAGAGGAAGCTGGTGGCAGGGCTGACTGCAAATGAGGGGAGCCAGTACAACCCACAG
GTGGCCGACCTGAAGGCCTTCTCCAAGCACATCTACAATGCCTACCTGAAAAACTTCAA
CATGACCAAAAAGAAGGCCCGCAGCATCCTCACCGGCAAAGCCAGCCACACGGCGCC
CTTTGTGATCCACGACATCGAGACATTGTGGCAGGCAGAGAAGGGGCTGGTGTGGAA
GCAGTTGGTGAATGGCCTGCCTCCCTACAAGGAGATCAGCGTGCACGTCTTCTACCGC
TGCCAGTGCACCACAGTGGAGACCGTGCGGGAGCTCACTGAGTTCGCCAAGAGCATC
CCCAGCTTCAGCAGCCTCTTCCTCAACGACCAGGTTACCCTTCTCAAGTATGGCGTGC
ACGAGGCCATCTTCGCCATGCTGGCCTCTATCGTCAACAAGGACGGGCTGCTGGTAG
CCAACGGCAGTGGCTTTGTCACCCGTGAGTTCCTGCGCAGCCTCCGCAAACCCTTCAG
TGATATCATTGAGCCTAAGTTTGAATTTGCTGTCAAGTTCAACGCCCTGGAACTTGATG
ACAGTGACCTGGCCCTATTCATTGCGGCCATCATTCTGTGTGGAGACCGGCCAGGCCT
CATGAACGTTCCACGGGTGGAGGCTATCCAGGACACCATCCTGCGTGCCCTCGAATTC
CACCTGCAGGCCAACCACCCTGATGCCCAGTACCTCTTCCCCAAGCTGCTGCAGAAGA
TGGCTGACCTGCGGCAACTGGTCACCGAGCACGCCCAGATGATGCAGCGGATCAAGA
AGACCGAAACCGAGACCTCGCTGCACCCTCTGCTCCAGGAGATCTACAAGGACATGTA
CTAATAGTGA (SEQ ID NO: 30)
Key for SEQ ID NO 30:
VP64 sequence (lower case font, bold, and underlined)
linker sequence (lower case font, bold, and italicized)
PPARδ SEQUENCE (CAPITALIZED FONT AND ITALICIZED)
Amino acid sequence encoding a VP64-PPAR8 fusion protein:
MDALDDFDLDMLGSDALDDEDLDMLGSDALDDFDLDMLGSDALDDFDLDMLG
GGGSMEQPQEEAPEVREEEEKEEVAEAEGAPELNGGPQHALPSSSYTDLSRSSSPPSLLD
QLQMGCDGASCGSLNMECRVCGDKASGFHYGVHACEGCKGFFRRTIRMKLEYEKCERS
CKIQKKNRNKCQYCRFQKCLALGMSHNAIRFGRMPEAEKRKLVAGLTANEGSQYNPQVA
DLKAFSKHIYNAYLKNFNMTKKKARSILTGKASHTAPFVIHDIETLWQAEKGLVWKQLVN
GLPPYKEISVHVFYRCQCTTVETVRELTEFAKSIPSFSSLFLNDQVTLLKYGVHEAIFAMLAS
IVNKDGLLVANGSGFVTREFLRSLRKPFSDIIEPKFEFAVKFNALELDDSDLALFIAAIILCG
DRPGLMNVPRVEAIQDTILRALEFHLQANHPDAQYLFPKLLQKMADLRQLVTEHAQMMQ
RIKKTETETSLHPLLQEIYKDMYDYKDDDDK (SEQ ID NO: 22)
Key for SEQ ID NO: 22:
VP64 sequence (bold and underlined)
linker sequence (bold, and italicized)
PPARδ SEQUENCE (ITALICIZED)
FLAG tag sequence (underlined)
Amino acid sequence encoding a VP64-PPAR8 fusion protein:
MDALDDFDLDMLGSDALDDFDLDMLGSDALDDFDLDMLGSDALDDEDLDMLG
GGGSMEQPQEEAPEVREEEEKEEVAEAEGAPELNGGPQHALPSSSYTDLSRSSSPPSLLD
QLQMGCDGASCGSLNMECRVCGDKASGFHYGVHACEGCKGFFRRTIRMKLEYEKCERS
CKIQKKNRNKCQYCRFQKCLALGMSHNAIRFGRMPEAEKRKLVAGLTANEGSQYNPQVA
DLKAFSKHIYNAYLKNFNMTKKKARSILTGKASHTAPFVIHDIETLWQAEKGLVWKQLVN
GLPPYKEISVHVFYRCQCTTVETVRELTEFAKSIPSFSSLFLNDQVTLLKYGVHEAIFAMLAS
IVNKDGLLVANGSGFVTREFLRSLRKPFSDIIEPKFEFAVKFNALELDDSDLALFIAAIILCG
DRPGLMNVPRVEAIQDTILRALEFHLQANHPDAQYLFPKLLQKMADLRQLVTEHAQMMQ
RIKKTETETSLHPLLQEIYKDMY (SEQ ID NO: 31)
Key for SEQ ID NO 31:
VP64 sequence (bold and underlined)
linker sequence (bold, and italicized)
PPARδ SEQUENCE (ITALICIZED)
Wild-type VP16 protein sequence:
MDLLVDELFADMNADGASPPPPRPAGGPKNTPAAPPLYATGRLSQAQLMPSPPMPV
PPAALFNRLLDDLGFSAGPALCTMLDTWNEDLFSALPTNADLYRECKFLSTLPSDVV
EWGDAYVPERTQIDIRAHGDVAFPTLPATRDGLGLYYEALSRFFHAELRAREESYRT
VLANFCSALYRYLRASVRQLHRQAHMRGRDRDLGEMLRATIADRYYRETARLARV
LFLHLYLFLTREILWAAYAEQMMRPDLFDCLCCDLESWRQLAGLFQPFMFVNGALT
VRGVPIEARRLRELNHIREHLNLPLVRSAATEEPGAPLTTPPTLHGNQARASGYFMVL
IRAKLDSYSSFTTSPSEAVMREHAYSRARTKNNYGSTIEGLLDLPDDDAPEEAGLAAP
RLSFLPAGHTRRLSTAPPTDVSLGDELHLDGEDVAMAHADALDDFDLDMLGDGDSP
GPGFTPHDSAPYGALDMADFEFEQMFTDALGIDEYGG (SEQ ID NO: 23)
Key for SEQ ID NO: 23:
Amino acids 411-490: Transcriptional activation (underlined)
Amino acids 446-490: transcriptional activation (Used in VP16-PPARd mouse disclosed
herein)

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of engineering an immune cell comprising introducing into an immune cell a fusion protein that comprises or consists of: (a) a peroxisome proliferator-activated receptor delta (PPAR-δ) sequence linked to (b) an activation domain sequence.

2. The method of claim 1, wherein the PPAR-δ sequence comprises or consists of a DNA binding domain and a ligand-binding domain.

3. The method of claim 1, wherein the PPAR-δ sequence comprises or consists of a sequence that is at least 90% identical to SEQ ID NO: 21 or SEQ ID NOs: 24-26.

4. The method of any one of claims 1-3, wherein the activation domain sequence is a Herpes simplex virion protein 16 (VP16) activation domain sequence.

5. The method of claim 1 or 4, wherein the method comprises introducing a nucleic acid sequence encoding the fusion protein.

6. The method of claim 5, wherein the nucleic acid sequence is present on an expression vector.

7. The method of claim 6, wherein the expression vector is a viral vector.

8. The method of any one of claims 1-7, wherein the PPAR-δ sequence is linked to the VP16 activation domain sequence via a peptide linker.

9. The method of claim 8, wherein the peptide linker is a poly-Glycine-Serine (G4S) linker.

10. The method of any one of claims 1-9, wherein the immune cell is a lymphoid cell or a myeloid cell.

11. The method of any one of claims 1-10, wherein the immune cell is a myeloid cell.

12. The method of claim 11, wherein the myeloid cell is a macrophage.

13. The method of any one of claims 1-10, wherein the immune cell is a lymphoid cell.

14. The method of claim 13, wherein the lymphoid cell is a T-cell or natural killer cell.

15. The method of claim 14, wherein the T-cell is a regulatory T cell or a cytotoxic T cell.

16. The method of claim 14 or 15, wherein the T-cell comprises a chimeric antigen receptor (CAR).

17. The method of claim 14 or 15, comprising introducing a chimeric antigen receptor (CAR) into the T-cell.

18. The method of claim 16 or 17, wherein the CAR comprises a HER-2 antibody.

19. The method of any one of claims 11-18, wherein the VP16 activation domain sequence comprises four copies of VP16.

20. The method of any one of claims 11-19, wherein the VP16 activation domain sequence is VP64.

21. The method of any one of claims 1-20, wherein the fusion protein comprises or consists of an amino acid sequence that is at least 90% identical to SEQ ID NO: 1, 14, 15, or 16.

22. The method of any one of claims 11-21, wherein the VP16 activation domain sequence is linked to the N-terminus of the PPAR-δ sequence.

23. The method of any one of claims 1-22, wherein the fusion protein is introduced in an amount sufficient to increase expression of carnitine palmitoyltransferase 1A (CPT1A) by at least 25% as compared to the immune cell without the fusion protein.

24. The method of any one of claims 1-23, wherein the fusion protein is introduced in an amount sufficient to increase expression of expression of a proinflammatory molecule selected from the group consisting of ISG15, IRF7, IRF1, IFIT3, IFI208, and CXCL10 by at least 25% as compared to the immune cell without the fusion protein.

25. The method of any one of claims 1-24, wherein the fusion protein is introduced in an amount sufficient to increase expression of CXCR3, CXCR1, CCR6, GZMK, GZMB, GZMF, HIST4H4, HIST1H4M, BCL2, FOS, and/or JUN by at least 25% as compared to the immune cell without the fusion protein.

26. The method of any one of claims 1-25, wherein the fusion protein is introduced in an amount sufficient to decrease expression of PDCD1, TIM-3 and LAG-3 by at least 25% as compared to the immune cell without the fusion protein.

27. The method of any one of claims 1-26, wherein the fusion protein is introduced in an amount sufficient to increase expression of TNF-α by at least 25% as compared to the immune cell without the fusion protein.

28. The method of any one of claims 1-27, wherein the fusion protein is introduced in an amount sufficient to decrease expression of FOXP3 by at least 25% and/or increase expression of IFNG and TBX21 (t-bet) by at least 25% as compared to the immune cell without the fusion protein.

29. The method of any one of claims 1-28, wherein the immune cell expresses CD8 and/or CXCR3.

30. The method of any one of claims 1-29, wherein the immune cell comprises a nucleotide sequence encoding CPT1A and/or CXCR3.

31. The method of any one of claims 1-30, wherein the fusion protein is introduced in an amount sufficient to increase infiltration of the immune cell into a tumor and/or increases the lifespan of the immune cell as compared to an immune cell that does not comprise the fusion protein.

32. A method of increasing T cell-mediated cytotoxicity comprising introducing into a T cell a fusion protein that comprises: (a) a peroxisome proliferator-activated receptor delta (PPAR-δ) linked to (b) an activation domain sequence.

33. The method of claim 32, wherein the PPAR-δ sequence comprises or consists of a DNA binding domain and a ligand-binding domain.

34. The method of claim 32, wherein the PPAR-δ sequence comprises or consists of a sequence that is at least 90% identical to SEQ ID NO: 21 or SEQ ID NOs: 24-26.

35. The method of any one of claims 32-34, wherein the activation domain sequence is a Herpes simplex virion protein 16 (VP16) activation domain sequence.

36. The method of claim 32 or 35, wherein the method comprises introducing a nucleic acid sequence encoding the fusion protein.

37. The method of claim 36, wherein the nucleic acid sequence is present on an expression vector.

38. The method of claim 37, wherein the expression vector is a viral vector.

39. The method of any one of claims 32-38, wherein the PPAR-δ sequence is linked to the VP16 activation domain sequence via a peptide linker.

40. The method of claim 39, wherein the peptide linker is a poly-Glycine-Serine (G4S) linker.

41. The method of any one of claims 32-40, wherein the T-cell is a regulatory T cell or a cytotoxic T cell.

42. The method of any one of claims 32-41, wherein the T-cell comprises a chimeric antigen receptor (CAR).

43. The method of any one of claims 32-41 comprising introducing a chimeric antigen receptor (CAR) into the T-cell.

44. The method of claim 42 or 43, wherein the CAR comprises a HER-2 antibody.

45. The method of any one of claims 35-44, wherein the VP16 activation domain sequence comprises four copies of VP16.

46. The method of any one of claims 35-45, wherein the VP16 activation domain sequence is VP64.

47. The method of any one of claims 32-46, wherein the fusion protein comprises or consists of an amino acid sequence that is at least 90% identical to SEQ ID NO: 1, 14, 15, or 16.

48. The method of any one of claims 35-47, wherein the VP16 activation domain sequence is linked to the N-terminus of the PPAR-δ sequence.

49. The method of any one of claims 32-48, wherein the fusion protein is introduced in an amount sufficient to increase expression of carnitine palmitoyltransferase 1A (Cpt1A) by at least 25% as compared to the T-cell without the fusion protein.

50. The method of any one of claims 32-49, wherein the fusion protein is introduced in an amount sufficient to increase expression of CXCR3, CXCR1, CCR6, GZMK, GZMB, GZMF, HIST4H4, HIST1H4M, BCL2, FOS, and/or JUN by at least 25% as compared to the T-cell without the fusion protein.

51. The method of any one of claims 32-50, wherein the fusion protein is introduced in an amount sufficient to decrease expression of PDCD1, TIM-3 and LAG-3 by at least 25% as compared to the T-cell without the fusion protein.

52. The method of any one of claims 32-51, wherein the fusion protein is introduced in an amount sufficient to increase expression of TNF-α by at least 25% as compared to the T-cell without the fusion protein.

53. The method of any one of claims 32-52, wherein the fusion protein is introduced in an amount sufficient to decrease expression of FOXP3 by at least 25% and/or increase expression of IFNG and TBX21 (t-bet) by at least 25% as compared to the T-cell without the fusion protein.

54. The method of any one of claims 32-53, wherein the T-cell expresses CD8 and/or CXCR3.

55. The method of any one of claims 32-54, wherein the T-cell comprises a nucleotide sequence encoding CPT1A and/or CXCR3.

56. The method of any one of claims 32-55, wherein the fusion protein is introduced in an amount sufficient to increase infiltration of the T-cell into a tumor and/or increases the lifespan of the T-cell as compared to a T-cell that does not comprise the fusion protein.

57. A method of treating a subject with cancer comprising administering to the subject an immune cell comprising a fusion protein that comprises: (a) a peroxisome proliferator-activated receptor delta (PPAR-δ) linked to (b) an activation domain sequence.

58. The method of claim 57, wherein the PPAR-δ sequence comprises or consists of a DNA binding domain and a ligand-binding domain.

59. The method of claim 57, wherein the PPAR-δ sequence comprises or consists of a sequence that is at least 90% identical to SEQ ID NO: 21 or SEQ ID NOs: 24-26.

60. The method of any one of claims 57-59, wherein the activation domain sequence is a Herpes simplex virion protein 16 (VP16) activation domain sequence.

61. The method of claim 57 or 60, wherein the cancer is melanoma, breast cancer, colon cancer, or ovarian cancer.

62. The method of any one of claims 57-61, wherein the immune cell is introduced in a number sufficient to decrease the size of a tumor in the subject by at least 25% as compared to when the immune cell is not introduced to the subject.

63. The method of any one of claims 57-62, wherein the immune cell is introduced in a number sufficient to increase the number of tumor-infiltrating immune cells in a tumor of the subject by 25% as compared to when the immune cell is not introduced to the subject.

64. The method of any one of claims 57-63, wherein the immune cell is introduced in a number sufficient to decrease the rate of metastasis in a subject by 25% as compared to when the immune cell is not introduced to the subject.

65. A method of engineering an immune cell comprising introducing into an immune cell a CPT1A protein.

66. A method of treating a subject with cancer comprising administering to the subject an immune cell comprising a CPT1A protein.

67. The method of claim 65 or 66, wherein the method comprises introducing a nucleic acid sequence encoding the CPT1A protein.

68. The method of claim 67, wherein the nucleic acid sequence is present on an expression vector.

69. The method of claim 68, wherein the expression vector is a viral vector.

70. The method of any one of claims 65-69, wherein the immune cell is a lymphoid cell or a myeloid cell.

71. The method of any one of claims 65-69, wherein the immune cell is a myeloid cell.

72. The method of claim 71, wherein the myeloid cell is a macrophage.

73. The method of any one of claims 65-70, wherein the immune cell is a lymphoid cell.

74. The method of claim 73, wherein the lymphoid cell is a T-cell or natural killer cell.

75. The method of claim 74, wherein the T-cell is a regulatory T cell or a cytotoxic T cell.

76. The method of claim 74 or 75, wherein the T-cell comprises a chimeric antigen receptor (CAR).

77. The method of claim 76, comprising introducing a chimeric antigen receptor (CAR) into the T-cell.

78. The method of claim 76 or 77, wherein the CAR comprises a HER-2 antibody.

79. The method of any one of claims 65-78, wherein the CPT1A protein comprises an amino acid sequence that is at least 90% identical to SEQ ID NO: 12.

80. The method of any one of claims 65-79, wherein the immune cell expresses CD8 and/or CXCR3.

81. The method of any one of claims 65-80, wherein the immune cell comprises a nucleotide sequence encoding or CXCR3.

82. The method of any one of claims 65-81, wherein the fusion protein is introduced in an amount sufficient to increase infiltration of the immune cell into a tumor and/or increases the lifespan of the immune cell as compared to an immune cell that does not comprise the fusion protein.

83. The method of any one of claims 66-82, wherein the cancer is melanoma, breast cancer, colon cancer, or ovarian cancer.

84. The method of any one of claims 66-83, wherein the immune cell is introduced in number sufficient to decrease the size of a tumor in the subject by at least 25% as compared to when the immune cell is not introduced to the subject.

85. The method of any one of claims 66-84, wherein the immune cell is introduced in a number sufficient to increase the number of tumor-infiltrating immune cells in a tumor of the subject by 10% as compared to when the immune cell is not introduced to the subject.

86. A method of increasing T cell-mediated cytotoxicity comprising introducing into a T-cell a CPT1A protein.

87. The method of claim 86, wherein the method comprises introducing a nucleic acid sequence encoding the CPT1A protein.

88. The method of claim 87, wherein the nucleic acid sequence is present on an expression vector.

89. The method of claim 88, wherein the expression vector is a viral vector.

90. The method of any one of claims 86-89, wherein the T-cell comprises a chimeric antigen receptor (CAR).

91. The method of claim 90, comprising introducing a chimeric antigen receptor (CAR) into the T-cell.

92. The method of claim 90 or 91, wherein the CAR comprises a HER-2 antibody.

93. The method of any one of claims 86-92, wherein the CPT1A protein comprises an amino acid sequence that is at least 90% identical to SEQ ID NO: 12.

94. The method of any one of claims 86-93, wherein the immune cell expresses CD8 and/or CXCR3.

95. The method of any one of claims 86-94, wherein the immune cell comprises a nucleotide sequence encoding or CXCR3.

96. The method of any one of claims 86-95, wherein the fusion protein is introduced in an amount sufficient to increase infiltration of the T-cell into a tumor and/or increases the lifespan of the immune cell as compared to an T-cell that does not comprise the fusion protein.

97. A T-cell comprising: (a) a chimeric antigen receptor and (b) a fusion protein that comprises or consists of: (i) a peroxisome proliferator-activated receptor delta (PPAR-δ) linked to (ii) an activation domain sequence.

98. A T-cell comprising: (a) a chimeric antigen receptor and (b) a fusion protein that comprises or consists of: (i) a peroxisome proliferator-activated receptor delta (PPAR-δ) linked to (ii) an activation domain sequence.

99. A T-cell comprising: (a) a chimeric antigen receptor and (b) an engineered polynucleotide encoding (i) a peroxisome proliferator-activated receptor delta (PPAR-δ) linked to (ii) an activation domain sequence or (c) a chimeric antigen receptor and (d) an engineered polynucleotide encoding a CPT1A.

100. The method of any one of claims 57-64, further comprising administering an immune checkpoint inhibitor to the subject.

101. The method of claim 100, wherein the immune checkpoint inhibitor is a PD-1 inhibitor, a CTLA-4 inhibitor, or a PD-L1 inhibitor.


SEQUENCE LISTING (39 sequences)

SEQ ID NO: 1 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 2 - protein, 440 aa, Mus musculus
MEQPQEETPEAREEEKEEVAMGDGAPELNGGPEHTLPSSSCADLSQNSSPSSLLDQLQMGCDGASGGSLNMECRVCGDKASGFHYGVHACEGCKGFFRRTIRMKLEYEKCDRICKIQKKNRNKCQYCRFQKCLALGMSHNAIRFGRMPEAEKRKLVAGLTASEGCQHNPQLADLKAFSKHIYNAYLKNFNMTKKKARSILTGKSSHNAPFVIHDIETLWQAEKGLVWKQLVNGLPPYNEISVHVFYRCQSTTVETVRELTEFAKNIPNFSSLFLNDQVTLLKYGVHEAIFAMLASIVNKDGLLVANGSGFVTHEFLRSLRKPFSDIIEPKFEFAVKFNALELDDSDLALFIAAIILCGDRPGLMNVPQVEAIQDTILRALEFHLQVNHPDSQYLFPKLLQKMADLRQLVTEHAQMMQWLKKTESETLLHPLLQEIYKDMY

SEQ ID NO: 3 - DNA, 1326 bp, Homo sapiens
  source 1..1326: mol_type = genomic DNA
atggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaatgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaa

SEQ ID NO: 4 - protein, 5 aa, synthetic construct
GGGGS

SEQ ID NO: 5 - DNA, 2322 bp, Rattus norvegicus
  source 1..2322: mol_type = genomic DNA
atggcagaggctcaccaagctgtggccttccagttcaccgtcacccccgatggcattgacctccgcctgagccacgaagccctcaaacagatctgcctgtcggggctgcactcctggaagaagaagttcatccggttcaagaatggcatcatcactggtgtgttccccgcgaatccgtccagctggcttatcgtggtggtgggtgtgatttcatccatgcatgccaaagtggacccctccctgggcatgatcgcaaagatcagtcggaccctagacaccactggccgcatgtcaagccagacgaagaacattgtgagcggcgtcctctttggtacagggctctgggtggcagtcatcatgaccatgcgctactcgctgaaggtgctgctctcctaccacggctggatgtttgcagaacacggcaaaatgagccgcagcaccaagatctggatggctatggtcaaggtcctctcaggtcggaagcccatgttgtacagcttccagacgtctctgccacgcctgcctgtcccagctgtcaaagatactgtgagcaggtacctggaatctatgaggccactgatgaaggaagaagacttccagcgcatgacagcactggcccaggattttgctgtcaacctcggacccaaattgcagtggtatttgaagctaaaatcctggtgggccacaaattacgtgagtgactggtgggaagaatatatctacctgcggggccgagggccgctcatggtcaacagcaactactacgccatggagatgctgtacatcaccccaacccatatccaggcagcgagagctggcaacaccatccacgccatactgctgtatcgtcgcacattagaccgtgaggaactcaaacccattcgtcttctgggatccaccattccactctgctcagcccagtgggagcgactcttcaatacttcccggatccctggggaggagacagacaccatccaacatatcaaggacagcaggcacattgttgtgtaccacagagggcggtacttcaaggtctggctctaccacgatgggaggctgctgaggccccgagagctggagcagcaaatgcagcagatcctggatgatccctcagagccacagcctggggaggccaagctggccgccctcactgctgcagacagagtgccctgggcaaagtgtcggcagacctattttgcacgagggaaaaataagcagtccctggatgcggtggaaaaggcagcgttcttcgtgacgttggacgaatcggagcagggatacagagaggaggatcctgaggcatccatcgacagctacgccaaatccctgctgcatggaagatgctttgacaggtggtttgacaagtccatcacctttgttgtcttcaaaaacagcaagataggcataaatgcagagcactcctgggcggacgcgcccatcgtgggccatttgtgggagtatgtcatggccaccgacgtcttccagctgggttactcagaggatggacactgtaaaggagacaccaaccccaacatccctaagcccacaaggctacaatgggacattccaggagagtgccaggaggtcatagatgcatccctgagcagcgccagtcttttggcaaatgatgtggacctgcattccttcccatttgactctttcggcaaaggcttgatcaagaagtgccggacgagtcccgatgccttcatccagctggcgctgcagctcgcacattacaaggacatgggcaagttctgcctcacatatgaggcctccagtacccggctcttccgagaagggaggacagagactgtacgctcctgcactatggagtcctgcaactttgtgcaggccatgatggaccccaagtcaacggcagagcagaggctcaagctgttcaagatagcttgtgagaagcaccagcacctgtaccgcctcgccatgacgggcgccggcatcgaccgccatctcttctgcctctatgtggtgtccaagtatcttgcagtcgactcacctttcctgaaggaggtattgtctgagccatggaggttgtctacgagccagactcctcagcagcaggtggagctctttgactttgagaaaaaccctgactatgtgtcctgtggagggggctttgggccggttgccgatgacggctatggtgtctcctacattatagtgggagagaattttatccacttccatatttcttccaagttctctagccctgagacagactcacaccgctttgggaagcacttgagacaagccatgatggacattatcaccttgtttggcctcaccatcaattctaaaaagtaa

SEQ ID NO: 6 - protein, 11 aa, synthetic construct
DALDDFDLDML

SEQ ID NO: 7 - protein, 50 aa, synthetic construct
DALDDFDLDMLGSDALDDFDLDMLGSDALDDFDLDMLGSDALDDFDLDML

SEQ ID NO: 8 - DNA, 150 bp, synthetic construct
  source 1..150: mol_type = other DNA
gatgctttagacgattttgacttagatatgcttggttcagacgcgttagacgacttcgacctagacatgttaggctcagatgcattggacgacttcgatttagatatgttgggctccgatgccctagatgacttcgacctggacatgctg

SEQ ID NO: 9 - protein, 6 aa, synthetic construct
GGGGGG

SEQ ID NO: 10 - protein, 8 aa, synthetic construct
GGGGGGGG

SEQ ID NO: 11 - protein, 46 aa, synthetic construct
AEAAAKEAAAKEAAAKEAAAKALEAEAAAKEAAAKEAAAKEAAAKA

SEQ ID NO: 12 - protein, 773 aa, Homo sapiens
MAEAHQAVAFQFTVTPDGIDLRLSHEALRQIYLSGLHSWKKKFIRFKNGIITGVYPASPSSWLIVVVGVMTTMYAKIDPSLGIIAKINRTLETANCMSSQTKNVVSGVLFGTGLWVALIVTMRYSLKVLLSYHGWMFTEHGKMSRATKIWMGMVKIFSGRKPMLYSFQTSLPRLPVPAVKDTVNRYLQSVRPLMKEEDFKRMTALAQDFAVGLGPRLQWYLKLKSWWATNYVSDWWEEYIYLRGRGPLMVNSNYYAMDLLYILPTHIQAARAGNAIHAILLYRRKLDREEIKPIRLLGSTIPLCSAQWERMFNTSRIPGEETDTIQHMRDSKHIVVYHRGRYFKVWLYHDGRLLKPREMEQQMQRILDNTSEPQPGEARLAALTAGDRVPWARCRQAYFGRGKNKQSLDAVEKAAFFVTLDETEEGYRSEDPDTSMDSYAKSLLHGRCYDRWFDKSFTFVVFKNGKMGLNAEHSWADAPIVAHLWEYVMSIDSLQLGYAEDGHCKGDINPNIPYPTRLQWDIPGECQEVIETSLNTANLLANDVDFHSFPFVAFGKGIIKKCRTSPDAFVQLALQLAHYKDMGKFCLTYEASMTRLFREGRTETVRSCTTESCDFVRAMVDPAQTVEQRLKLFKLASEKHQHMYRLAMTGSGIDRHLFCLYVVSKYLAVESPFLKEVLSEPWRLSTSQTPQQQVELFDLENNPEYVSSGGGFGPVADDGYGVSYILVGENLINFHISSKFSCPETDSHRFGRHLKEAMTDIITLFGLSSNSKK

SEQ ID NO: 13 - protein, 773 aa, Rattus norvegicus
MAEAHQAVAFQFTVTPDGIDLRLSHEALKQICLSGLHSWKKKFIRFKNGIITGVFPANPSSWLIVVVGVISSMHAKVDPSLGMIAKISRTLDTTGRMSSQTKNIVSGVLFGTGLWVAVIMTMRYSLKVLLSYHGWMFAEHGKMSRSTKIWMAMVKVLSGRKPMLYSFQTSLPRLPVPAVKDTVSRYLESMRPLMKEEDFQRMTALAQDFAVNLGPKLQWYLKLKSWWATNYVSDWWEEYIYLRGRGPLMVNSNYYAMEMLYITPTHIQAARAGNTIHAILLYRRTLDREELKPIRLLGSTIPLCSAQWERLFNTSRIPGEETDTIQHIKDSRHIVVYHRGRYFKVWLYHDGRLLRPRELEQQMQQILDDPSEPQPGEAKLAALTAADRVPWAKCRQTYFARGKNKQSLDAVEKAAFFVTLDESEQGYREEDPEASIDSYAKSLLHGRCFDRWFDKSITFVVFKNSKIGINAEHSWADAPIVGHLWEYVMATDVFQLGYSEDGHCKGDTNPNIPKPTRLQWDIPGECQEVIDASLSSASLLANDVDLHSFPFDSFGKGLIKKCRTSPDAFIQLALQLAHYKDMGKFCLTYEASSTRLFREGRTETVRSCTMESCNFVQAMMDPKSTAEQRLKLFKIACEKHQHLYRLAMTGAGIDRHLFCLYVVSKYLAVDSPFLKEVLSEPWRLSTSQTPQQQVELFDFEKNPDYVSCGGGFGPVADDGYGVSYIIVGENFIHFHISSKFSSPETDSHRFGKHLRQAMMDIITLFGLTINSKK

SEQ ID NO: 14 - protein, 361 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGGE

SEQ ID NO: 15 - protein, 402 aa, Homo sapiens
MHQRDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 16 - protein, 343 aa, Homo sapiens
MEQPQEEAPEVREEEEKEEVAEAEGAPELNGGPQHALPSSSYTAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 17 - DNA, 3734 bp, Homo sapiens
  source 1..3734: mol_type = genomic DNA
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaa

SEQ ID NO: 18 - DNA, 15 bp, synthetic construct
  source 1..15: mol_type = other DNA
ggaggaggaggtagt

SEQ ID NO: 19 - DNA, 1320 bp, synthetic construct
  source 1..1320: mol_type = other DNA
gagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaatgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtac

SEQ ID NO: 20 - DNA, 1524 bp, synthetic construct
  source 1..1524: mol_type = other DNA
atggatgctttagacgattttgacttagatatgcttggttcagacgcgttagacgacttcgacctagacatgttaggctcagatgcattggacgacttcgatttagatatgttgggctccgatgccctagatgacttcgacctggacatgctgggaggaggaggtagtatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaatgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtacgattataaagatgatgatgataaataatagtga

SEQ ID NO: 21 - protein, 440 aa, synthetic construct
EQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 22 - protein, 505 aa, synthetic construct
MDALDDFDLDMLGSDALDDFDLDMLGSDALDDFDLDMLGSDALDDFDLDMLGGGGSMEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMYDYKDDDDK

SEQ ID NO: 23 - protein, 490 aa, Human alphaherpesvirus 1
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 24 - protein, 54 aa, synthetic construct
MEQPQEEAPEVREEEEKEEVAEAEGAPELNGGPQHALPSSSYTDLSRSSSPPSL

SEQ ID NO: 25 - protein, 75 aa, synthetic construct
NMECRVCGDKASGFHYGVHACEGCKGFFRRTIRMKLEYEKCERSCKIQKKNRNKCQYCRFQKCLALGMSHNAIRF

SEQ ID NO: 26 - protein, 229 aa, synthetic construct
FVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKD

SEQ ID NO: 27 - DNA, 5238 bp, Rattus norvegicus
  source 1..5238: mol_type = genomic DNA
aatccgctgctgccggcgtcgggtgcgctcggcctcgcccgcggccctccttccccggctcccgctcgccgctcgttcactccaccgccgccgccgccgccgccgctgccgctgccgctgccgcacctccgtagctgactcggtactctctgaagatggcagaagctcaccaagctgtggcctttcagttcacggtcactccggacgggattgacctgcggctgagccatgaagctcttagacaaatctatctctctggacttcattcctggaaaaagaagttcatcagattcaagaacggcatcatcactggcgtgtacccggcaagcccctccagttggcttatcgtggtggtgggcgtgatgacaacgatgtacgccaagatcgacccctcgttaggaataattgcaaaaatcaatcggactctggaaacggccaactgcatgtccagccagacgaagaacgtggtcagcggcgtgctgtttggcaccggcctgtgggtggccctcatcgtcaccatgcgctactccctgaaagtgctgctctcctaccacgggtggatgttcactgagcacggcaagatgagtcgtgccaccaagatctggatgggtatggtcaagatcttttcaggccgaaaacccatgttgtacagcttccagacatcgctgcctcgcctgccggtcccggctgtcaaagacactgtgaacaggtatctacagtcggtgaggcctcttatgaaggaagaagacttcaaacggatgacagcacttgctcaagattttgctgtcggtcttggaccaagattacagtggtatttgaagttaaaatcctggtgggctacaaattacgtgagcgactggtgggaggagtacatctacctccgaggacgagggccgctcatggtgaacagcaactattatgccatggatctgctgtatatccttccaactcacattcaggcagcaagagccggcaacgccatccatgccatcctgctttacaggcgcaaactggaccgggaggaaatcaaaccaattcgtcttttgggatccacgattccactctgctccgctcagtgggagcggatgtttaatacttcccggatcccaggagaggagacagacaccatccagcacatgagagacagcaagcacatcgtcgtgtaccatcgaggacgctacttcaaggtctggctctaccatgatgggcggctgctgaagccccgggagatggagcagcagatgcagaggatcctggacaatacctcggagcctcagcccggggaggccaggctggcagccctcaccgcaggagacagagttccctgggccaggtgtcgtcaggcctattttggacgtgggaaaaataagcagtctcttgatgctgtggagaaagcagcgttctttgtgacgttagatgaaactgaagaaggatacagaagtgaagacccggatacgtcaatggacagctacgccaaatctctactacacggccgatgttacgacaggtggtttgacaagtcgttcacgtttgttgtcttcaaaaacgggaagatgggcctcaacgctgaacactcctgggcagatgcgccgatcgtggcccacctttgggagtacgtcatgtccattgacagcctccagctgggctatgcggaggatgggcactgcaaaggcgacatcaatccgaacattccgtaccccaccaggctgcagtgggacatcccgggggaatgtcaagaggttatagagacctccctgaacaccgcaaatcttctggcaaacgacgtggatttccattccttcccattcgtagcctttggtaaaggaatcatcaagaaatgtcgcacgagcccagacgcctttgtgcagctggccctccagctggcgcactacaaggacatgggcaagttttgcctcacatacgaggcctccatgacccggctcttccgagaggggaggacggagaccgtgcgctcctgcaccactgagtcatgcgacttcgtgcgggccatggtggacccggcccagacggtggaacagaggctgaagttgttcaagttggcgtctgagaagcatcagcatatgtatcgcctcgccatgaccggctctgggatcgatcgtcacctcttctgcctttacgtggtgtctaaatatctcgctgtggagtcccctttccttaaggaagttttatctgagccttggagattatcaacaagccagacccctcagcagcaagtggagctgtttgacttggagaataacccagagtacgtgtccagcggagggggctttggaccggttgctgatgacggctatggtgtgtcgtacatccttgtgggagagaacctcatcaatttccacatttcttccaagttctcttgccctgagacggattctcatcgctttggaaggcacctgaaagaagcaatgactgacatcatcactttgtttggtctcagttctaattccaaaaagtaattccactggagctgctgggaaggaaaacgagctcttctgatgcaaaccaaatgaaaaataggcattaatcctgaccttagctcgggatgaaacactgctcttaaaaaaactcagttttccttccagaaaatgtgggtgtttttttttcctagaacagtatctctcccctgtgaagcataaccccactacttccagacttgccctcccttgggggacatctgataaagtctcccctgatgtctccgcatcggcttggatttattaagggatgcaaatcttgttgagttaatgaaggaattagtagggttgtggcttcacacacagtggaatggaaatggtgtgctttctcagtggcaaccgaaggcctagtgcttaagggcatttagcatcatccaagcagggtaaacttttgttttgttaaaagaaaaatgtgttattcaagttggtgtccccagttgtagctaacacatctggaatgcactaaccaaaatgctgtgctttggagacctgcttttgtcaccgtgggtaaccgttcccgtctggtccagtagcctgtgtttgcctctccacatttgaagcaagcaggatgcaaggtcttcagttttactgaccttgtatgtcttcaagtcttcacaacccagtgccttaaaaatgaaaggccctaaatgtaagggagatggagagaaagatttattttgtagagtctttgggtggaattgtgggtatactgttcccttcacaattgactgagtatggataaccgtacataagcatttgctacaccccaccagccccctccccctcagaaacaccagttccttcccaagggcagctgtgccagactcccctcccgggactgccttcttgtcatcataagcaacaaaagaaataacaggcacatgtcataaaaggggagcaagggccgtgatggtcagataattcactcaagaataaaacatgacacgtgcctcaggaggatctctttcccaaagtgacagcaaggagggcagggcatcggccaccaagcggggactagcaagtgaggaaggggagggcagcccaccgtggtgaggagagagtggctccacgaccccaagggatggccttctcctcccacccggtgaggggaaagactcaccagagggtgatggagacagtatgccggctcaccttggtgaccagccaagatgtctcaagtgacagtgctaggtgttcacccagcctgtccttcagataggagtgccttcacgaaagcgtctcatggaccacaaagcaattatgcactgagtcatcttcagtatttaatgcaaaaatgaagcatcatggaatgaaattcccactgtctgtcatgacaagcttagctgtccattgttttaaattgtgtatttatttttttgaccacttggttctagttgggcctgactccttcagagtgctgcaccccgatagtacaacagcgatggctgaactgttggagtcgatggaaggtgcttgccggagaacacgtgcctttttttttttttttttttttgagatggagtttcactcttgttgcccaggctggagtgcagtggtgcaatctcggctcactgcagcatctgccttgcaggttcaagcgattctcctgtctcagcctcccaagtagctgggattacacgcccaacaccatgccctgctaatttttgtatttttagtagagacggggtttcatcatattggtcaggctggtctcgaactcctgacctcaggtgatccacctgcctcagcctcccaaagtgctgggattacaggcatgagccaccatgcgcggcccacatgcatgttttatgtatgtatacttcatgatgtaaaaatcccacctttatgggccaaagattttttttctcctgaaagcaagaaaaaatgaaaacaaaagacaaaaaaaaaaaaaaaagcgtccaggcgcggtggctcatgcgtgtaatcccagcactttgggaggccaaggcgggcggatcacgaggtcaggagatggagaccatcctggctaacacggtgaaaccccgtctctactaaaaattcaaaaaatgagccgggcgtggtggcgggcgcctgtagtcccagctactcaggaggctgaggcaggagaatggcgtgaacccgggaggcggagcttgcagtgagccgagatcgctccactgcactccagcctgggcgacagagcgagactccgtctaaaaaaaaaagcaaaaacaaaccaacaacaaaagcccctgactgtccgtcaagcaggcagcggggatgtagctctctctgccctgggcaagaatagcacttcccgttaaaagccagcagccggcgtcagtccctatcagagccagctagatcatgcactgttgaccactgagcaatctgtgttacactagagttcacagggcattttgagtgtagacgtgagtgcttaaacatatttgggtttctctctcaggttttaaatgtttcaaatgtaattgttgctcatcagtgcagttatcaatgcaattttatattccttgaggggagaaagaggggtcttattgtacatgtccaaggggggtgataagagtattatctgtttaatttaattggaacaaaccattgtcttaacgcagccatggtttgaatttgttatcttgggctgaccggtgcatgtaaatacagtatgctctttggatgtaaatcttagaaatgcagtgtgaatgtaggttatcattaataaaacattaaccccagtctactacaa

SEQ ID NO: 28 - protein, 773 aa, Rattus norvegicus
MAEAHQAVAFQFTVTPDGIDLRLSHEALKQICLSGLHSWKKKFIRFKNGIITGVFPANPSSWLIVVVGVISSMHAKVDPSLGMIAKISRTLDTTGRMSSQTKNIVSGVLFGTGLWVAVIMTMRYSLKVLLSYHGWMFAEHGKMSRSTKIWMAMVKVLSGRKPMLYSFQTSLPRLPVPAVKDTVSRYLESMRPLMKEEDFQRMTALAQDFAVNLGPKLQWYLKLKSWWATNYVSDWWEEYIYLRGRGPLMVNSNYYAMEMLYITPTHIQAARAGNTIHAILLYRRTLDREELKPIRLLGSTIPLCSAQWERLFNTSRIPGEETDTIQHIKDSRHIVVYHRGRYFKVWLYHDGRLLRPRELEQQMQQILDDPSEPQPGEAKLAALTAADRVPWAKCRQTYFARGKNKQSLDAVEKAAFFVTLDESEQGYREEDPEASIDSYAKSLLHGRCFDRWFDKSITFVVFKNSKIGINAEHSWADAPIVGHLWEYVMATDVFQLGYSEDGHCKGDTNPNIPKPTRLQWDIPGECQEVIDASLSSASLLANDVDLHSFPFDSFGKGLIKKCRTSPDAFIQLALQLAHYKDMGKFCLTYEASMTRLFREGRTETVRSCTMESCNFVQAMMDPKSTAEQRLKLFKIACEKHQHLYRLAMTGAGIDRHLFCLYVVSKYLAVDSPFLKEVLSEPWRLSTSQTPQQQVELFDFEKNPDYVSCGGGFGPVADDGYGVSYIIVGENFIHFHISSKFSSPETDSHRFGKHLRQAMMDIITLFGLTINSKK

SEQ ID NO: 29 - protein, 773 aa, synthetic construct
MAEAHQAVAFQFTVTPDGIDLRLSHEALKQICLSGLHSWKKKFIRFKNGIITGVFPANPSSWLIVVVGVISSMHAKVDPSLGMIAKISRTLDTTGRMSSQTKNIVSGVLFGTGLWVAVIMTMRYSLKVLLSYHGWMFAEHGKMSRSTKIWMAMVKVLSGRKPMLYSFQTSLPRLPVPAVKDTVSRYLESVRPLMKEEDFQRMTALAQDFAVNLGPKLQWYLKLKSWWATNYVSDWWEEYIYLRGRGPLMVNSNYYAMEMLYITPTHIQAARAGNTIHAILLYRRTLDREELKPIRLLGSTIPLCSAQWERLFNTSRIPGEETDTIQHIKDSRHIVVYHRGRYFKVWLYHDGRLLRPRELEQQMQQILDDPSEPQPGEAKLAALTAADRVPWAKCRQTYFARGKNKQSLDAVEKAAFFVTLDESEQGYREEDPEASIDSYAKSLLHGRCFDRWFDKSITFVVFKNSKIGINAEHSWADAPVVGHLWEYVMATDVFQLGYSEDGHCKGDTNPNIPKPTRLQWDIPGECQEVIDASLSSASLLANDVDLHSFPFDSFGKGLIKKCRTSPDAFIQLALQLAHYKDMGKFCLTYEASMTRLFREGRTETVRSCTMESCNFVQAMMDPKSTAEQRLKLFKIACEKHQHLYRLAMTGAGIDRHLFCLYVVSKYLAVDSPFLKEVLSEPWRLSTSQTPQQQVELFDFEKNPDYVSCGGGFGPVADDGYGVSYIIVGENFIHFHISSKFSSPETDSHRFGKHLRQAMMDIITLFGLTINSKK

SEQ ID NO: 30 - DNA, 1500 bp, synthetic construct
  source 1..1500: mol_type = other DNA
atggatgctttagacgattttgacttagatatgcttggttcagacgcgttagacgacttcgacctagacatgttaggctcagatgcattggacgacttcgatttagatatgttgggctccgatgccctagatgacttcgacctggacatgctgggaggaggaggtagtatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaatgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaatagtga

SEQ ID NO: 31 - protein, 497 aa, synthetic construct
MDALDDFDLDMLGSDALDDFDLDMLGSDALDDFDLDMLGSDALDDFDLDMLGGGGSMEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 32 - protein, 10 aa, synthetic construct
GGGGSGGGGS

SEQ ID NO: 33 - protein, 15 aa, synthetic construct
GGGGSGGGGSGGGGS

SEQ ID NO: 34 - protein, 20 aa, synthetic construct
GGGGSGGGGSGGGGSGGGGS

SEQ ID NO: 35 - protein, 5 aa, synthetic construct
EAAAK

SEQ ID NO: 36 - protein, 10 aa, synthetic construct
EAAAKEAAAK

SEQ ID NO: 37 - protein, 15 aa, synthetic construct
EAAAKEAAAKEAAAK

SEQ ID NO: 38 - protein, 5 aa, synthetic construct
PAPAP

SEQ ID NO: 39 - protein, 12 aa, synthetic construct
AEAAAKEAAAKA